SEQUENCE LISTING (821 sequences)

SEQ ID NO: 1 - protein, 35 aa, Haplopelma schmidti
ECLEIFKACNPSNDQCCKSSKLVCSRKTRWCKYQI

SEQ ID NO: 2 - protein, 35 aa, Haplopelma hainanum
ECLGFGKGCNPSNDQCCKSSNLVCSRKHRWCKYEI

SEQ ID NO: 3 - protein, 35 aa, Haplopelma hainanum
ECLGFGKGCNPSNDQCCKSANLVCSRKHRWCKYEI

SEQ ID NO: 4 - protein, 34 aa, Paraphysa scrofa
DCLGFLWKCNPSNDKCCRPNLVCSRKDKWCKYQI

SEQ ID NO: 5 - protein, 33 aa, Ceratogyrus marshalli
DCLGWFKSCDPKNDKCCKNYTCSRRDRWCKYDL

SEQ ID NO: 6 - protein, 33 aa, Ceratogyrus marshalli
DCLGWFKSCDPKNDKCCKNYTCSRRDRWCKYYL

SEQ ID NO: 7 - protein, 34 aa, Grammostola rosea
DCLGWFKGCDPDNDKCCEGYKCNRRDKWCKYKLW

SEQ ID NO: 8 - protein, 35 aa, Theraphosa blondi
AACLGMFESCDPNNDKCCPNRECNRKHKWCKYKLW

SEQ ID NO: 9 - protein, 35 aa, Chilobrachys guangxiensis
DDCLGMFSSCNPDNDKCCEGRKCDRRDQWCKWNPW

SEQ ID NO: 10 - protein, 33 aa, Chilobrachys guangxiensis
DCLGLFWICNYMDDKCCPGYKCERSSPWCKIDI

SEQ ID NO: 11 - protein, 35 aa, Theraphosa blondi
DDCLGMFSSCDPNNDKCCPNRVCRVRDQWCKYKLW

SEQ ID NO: 12 - protein, 35 aa, Theraphosa blondi
DDCLGMFSSCDPKNDKCCPNRVCRSRDQWCKYKLW

SEQ ID NO: 13 - protein, 33 aa, Haplopelma schmidti
ACKGVFDACTPGKNECCPNRVCSDKHKWCKWKL

SEQ ID NO: 14 - protein, 36 aa, Grammostola rosea
GCLEFWWKCNPNDDKCCRPKLKCSKLFKLCNFSFGK

SEQ ID NO: 15 - protein, 36 aa, Hadronyche versuta
ADCGWLFHSCESNADCCENWACATTGRFRYLCKYQI

SEQ ID NO: 16 - protein, 33 aa, Pandinus imperator
GDCLPHLKRCKADNDCCGKKCKRRGTNAEKRCR

SEQ ID NO: 17 - protein, 34 aa, Grammostola rosea
ECGKFMWKCKNSNDCCKDLVCSSRWKWCVLASPF

SEQ ID NO: 18 - protein, 33 aa, Haplopelma hainanum
ECKGFGKSCVPGKNECCSGYACNSRDKWCKVLL

SEQ ID NO: 19 - protein, 33 aa, unidentified
  source 1..33: note = Scorpio maurus palmatus
GDCLPHLKLCKENKDCCSKKCKRRGTNIEKRCR

SEQ ID NO: 20 - protein, 31 aa, Heteropoda venatoria
ECGTLFSGCSTHADCCEGFICKLWCRYERTW

SEQ ID NO: 21 - protein, 36 aa, Haplopelma hainanum
ECRYWLGTCSKTGDCCSHLSCSPKHGWCVWDWTFRK

SEQ ID NO: 22 - protein, 32 aa, Chilobrachys guangxiensis
GCQKFFWTCHPGQPPCCSGLACTWPTEICIDG

SEQ ID NO: 23 - protein, 33 aa, Haplopelma hainanum
GCKGFGDSCTPGKNECCPNYACSSKHKWCKVYL

SEQ ID NO: 24 - protein, 29 aa, Heteropoda venatoria
DDCGTLFSGCDTSKDCCEGYVCHLWCKYK

SEQ ID NO: 25 - protein, 34 aa, Stromatopelma calceatum
DCTRMFGACRRDSDCCPHLGCKPTSKYCAWDGTI

SEQ ID NO: 26 - protein, 34 aa, Chilobrachys guangxiensis
RCIEEGKWCPKKAPCCGRLECKGPSPKQKKCTRP

SEQ ID NO: 27 - protein, 35 aa, Thrixopelma pruriens
ECRYWLGGCSAGQTCCKHLVCSRRHGWCVWDGTFS

SEQ ID NO: 28 - protein, 35 aa, Heteroscodra maculata
ECRYLFGGCSSTSDCCKHLSCRSDWKYCAWDGTFS

SEQ ID NO: 29 - protein, 36 aa, Chilobrachys guangxiensis
EGECGGFWWKCGSGKPACCPKYVCSPKWGLCNFPMP

SEQ ID NO: 30 - protein, 36 aa, Chilobrachys guangxiensis
DGECGGFWWKCGSGKPACCPKYVCSPKWGLCNFPMP

SEQ ID NO: 31 - protein, 37 aa, Hadronyche versuta
DDDCGWIMDDCTSDSDCCPNWVCSKTGFVKNICKYEM

SEQ ID NO: 32 - protein, 31 aa, Chilobrachys guangxiensis
LCSREGEFCYKLRKCCAGFYCKAFVLHCYRN

SEQ ID NO: 33 - protein, 32 aa, Brachypelma smithii
SFCIPFKPCKSDENCCKKFKCKTTGIVKLCRW

SEQ ID NO: 34 - protein, 33 aa, Chilobrachys guangxiensis
ECRKMFGGCSVDSDCCAHLGCKPTLKYCAWDGT

SEQ ID NO: 35 - protein, 33 aa, Heteropoda venatoria
DCGTIWHYCGTDQSECCEGWKCSRQLCKYVIDW

SEQ ID NO: 36 - protein, 34 aa, Haplopelma schmidti
GCLGDKCDYNNGCCSGYVCSRTWKWCVLAGPWRR

SEQ ID NO: 37 - protein, 34 aa, Chilobrachys guangxiensis
GCGGLMAGCDGKSTFCCSGYNCSPTWKWCVYARP

SEQ ID NO: 38 - protein, 34 aa, Chilobrachys guangxiensis
ECRKMFGGCSVHSDCCAHLGCKPTLKYCAWDGTF

SEQ ID NO: 39 - protein, 34 aa, Chilobrachys guangxiensis
GCGGLMDGCDGKSTFCCSGFNCSPTWKWCVYARP

SEQ ID NO: 40 - protein, 30 aa, Heteropoda venatoria
DDCGGLFSGCDSNADCCEGYVCRLWCKYKL

SEQ ID NO: 41 - protein, 35 aa, Grammostola rosea
ECRYLFGGCKTTSDCCKHLGCKFRDKYCAWDFTFS

SEQ ID NO: 42 - protein, 35 aa, Grammostola rosea
ECRYLFGGCKTTADCCKHLGCKFRDKYCAWDFTFS

SEQ ID NO: 43 - protein, 35 aa, Psalmopoeus cambridgei
SECRWFMGGCDSTLDCCKHLSCKMGLYYCAWDGTF

SEQ ID NO: 44 - protein, 36 aa, Chilobrachys guangxiensis
ECRKMFGGCSVDSDCCAHLGCKPTLKYCAWDGTFGK

SEQ ID NO: 45 - protein, 38 aa, Heteroscodra maculata
ECRYFWGECNDEMVCCEHLVCKEKWPITYKICVWDRTF

SEQ ID NO: 46 - protein, 36 aa, Chilobrachys guangxiensis
DGECGGFWWKCGRGKPPCCKGYACSKTWGWCAVEAP

SEQ ID NO: 47 - protein, 40 aa, Psalmopoeus cambridgei
EDCIPKWKGCVNRHGDCCEGLECWKRRRSFEVCVPKTPKT

SEQ ID NO: 48 - protein, 35 aa, Allagelena opulenta
GGCLPHNRFCNALSGPRCCSGLKCKELSIWDSRCL

SEQ ID NO: 49 - protein, 37 aa, Chilobrachys guangxiensis
QCGEFMWKCGAGKPTCCSGYDCSPTWKWCVLKSPGRR

SEQ ID NO: 50 - protein, 36 aa, Chilobrachys guangxiensis
TCYDIGELCSSDKPCCSGYYCSPRWGWCIYSTRGGR

SEQ ID NO: 51 - protein, 37 aa, Hadronyche versuta
SAVCIPSGQPCPYSKYCCSGSCTYKTNENGNSVQRCD

SEQ ID NO: 52 - protein, 42 aa, Haplopelma hainanum
CAAEGIPCDPNPVKDLPCCSGLACLKPTLHGIWYKHHYCYTQ

SEQ ID NO: 53 - protein, 37 aa, Mesobuthus eupeus
GCNRKNKKCNSDADCCRYGERCISTKVNYYCRPDRGP

SEQ ID NO: 54 - protein, 32 aa, Chilobrachys guangxiensis
VCRGYGLPCTPEKNDCCQRLYCSQHRLCSVKA

SEQ ID NO: 55 - protein, 28 aa, Haplopelma schmidti
KCLPPGKPCYGATQKIPCCGVCSHNKCT

SEQ ID NO: 56 - protein, 28 aa, Chilobrachys guangxiensis
CGGWMAKCADSDDCCETFHCTRFNVCGK

SEQ ID NO: 57 - protein, 28 aa, Macrothele gigas
SCKLTFWRCKKDKECCGWNICTGLCIPP

SEQ ID NO: 58 - protein, 34 aa, unidentified
  source 1..34: note = Stromatopelma calceatum griseipes
TCRYLFGGCKTTADCCKHLACRSDGKYCAWDGTF

SEQ ID NO: 59 - protein, 34 aa, Chilobrachys guangxiensis
ECKWYLGDCKAHEDCCEHLRCHSRWDWCIWDGTF

SEQ ID NO: 60 - protein, 34 aa, Haplopelma hainanum
ECKYLWGTCEKDEHCCEHLGCNKKHGWCGWDGTF

SEQ ID NO: 61 - protein, 29 aa, Grammostola rosea
YCQKWLWTCDSERKCCEDMVCRLWCKKRL

SEQ ID NO: 62 - protein, 29 aa, Grammostola rosea
YCQKWMWTCDSKRKCCEDMVCQLWCKKRL

SEQ ID NO: 63 - protein, 29 aa, Paraphysa scrofa
YCQKWMWTCDSARKCCEGLVCRLWCKKII

SEQ ID NO: 64 - protein, 29 aa, Macrothele gigas
GCKLTFWKCKNKKECCGWNACALGICMPR

SEQ ID NO: 65 - protein, 36 aa, Haplopelma schmidti
ECRWYLGGCSQDGDCCKHLQCHSNYEWCVWDGTFSK

SEQ ID NO: 66 - protein, 35 aa, Psalmopoeus cambridgei
GACRWFLGGCKSTSDCCEHLSCKMGLDYCAWDGTF

SEQ ID NO: 67 - protein, 41 aa, Hysterocrates gigas
GVDKAGCRYMFGGCSVNDDCCPRLGCHSLFSYCAWDLTFSD

SEQ ID NO: 68 - protein, 35 aa, Conus pennaceus
DCTSWFGRCTVNSECCSNSCDQTYCELYAFPSFGA

SEQ ID NO: 69 - protein, 36 aa, Phoneutria nigriventer
IACAPRFSLCNSDKECCKGLRCQSRIANMWPTFCSQ

SEQ ID NO: 70 - protein, 36 aa, Phoneutria reidyi
IACAPRGLLCFRDKECCKGLTCKGRFVNTWPTFCLV

SEQ ID NO: 71 - protein, 36 aa, Chilobrachys guangxiensis
DCRKMFGGCSKHEDCCAHLACKRTFNYCAWDGSFSK

SEQ ID NO: 72 - protein, 35 aa, Chilobrachys guangxiensis
ECRWLFGGCEKDSDCCEHLGCRRAKPSWCGWDFTV

SEQ ID NO: 73 - protein, 35 aa, Chilobrachys guangxiensis
ECRWLFGGCEKDSDCCEHLGCRRAKPSWCGWDFTF

SEQ ID NO: 74 - protein, 36 aa, Phoneutria nigriventer
IACAPRFSICNSDKECCKGLRCQSRIANMWPTFCLV

SEQ ID NO: 75 - protein, 39 aa, Haplopelma hainanum
CIGEGVPCDENDPRCCSGLVCLKPTLHGIWYKSYYCYKK

SEQ ID NO: 76 - protein, 35 aa, Haplopelma hainanum
DCAGYMRECKEKLCCSGYVCSSRWKWCVLPAPWRR

SEQ ID NO: 77 - protein, 36 aa, Haplopelma hainanum
ECRWYLGGCSQDGDCCKHLQCHSNYEWCIWDGTFSK

SEQ ID NO: 78 - protein, 35 aa, Chilobrachys guangxiensis
ECKKLFGGCTTSSECCAHLGCKQKWPFYCAWDWSF

SEQ ID NO: 79 - protein, 40 aa, Heriaeus melloteei
GCIPSFGECAWFSGESCCTGICKWVFFTSKFMCRRVWGKD

SEQ ID NO: 80 - protein, 35 aa, Hadrurus gertschi
SEKDCIKHLQRCRENKDCCSKKCSRRGTNPEKRCR

SEQ ID NO: 81 - protein, 30 aa, Thrixopelma pruriens
YCQKWMWTCDSERKCCEGMVCRLWCKKKLW

SEQ ID NO: 82 - protein, 30 aa, Chilobrachys guangxiensis
YCQKWMWTCDSKRACCEGLRCKLWCRKIIG

SEQ ID NO: 83 - protein, 30 aa, Heteropoda venatoria
DDCGKLFSGCDTNADCCEGYVCRLWCKLDW

SEQ ID NO: 84 - protein, 31 aa, Grammostola rosea
YCQKWMWTCDEERKCCEGLVCRLWCKKKIEW

SEQ ID NO: 85 - protein, 31 aa, Conus marmoreus
ACSKKWEYCIVPILGFVYCCPGLICGPFVCV

SEQ ID NO: 86 - protein, 31 aa, Grammostola rosea
YCQKWMWTCDEERKCCEGLVCRLWCKRIINM

SEQ ID NO: 87 - protein, 32 aa, Grammostola rosea
YCQKWMWTCDEERKCCEGLVCRLWCKKKIEEG

SEQ ID NO: 88 - protein, 32 aa, Chilobrachys guangxiensis
GCGTMWSPCSTEKPCCDNFSCQPAIKWCIWSP

SEQ ID NO: 89 - protein, 34 aa, Psalmopoeus cambridgei
ECRWYLGGCKEDSECCEHLQCHSYWEWCLWDGSF

SEQ ID NO: 90 - protein, 39 aa, Ceratogyrus marshalli
GVDKEGCRKLLGGCTIDDDCCPHLGCNKKYWHCGWDGTF

SEQ ID NO: 91 - protein, 36 aa, Chilobrachys guangxiensis
ECTKFLGGCSEDSECCPHLGCKDVLYYCAWDGTFGK

SEQ ID NO: 92 - protein, 35 aa, Chilobrachys guangxiensis
ECTKLLGGCTKDSECCPHLGCRKKWPYHCGWDGTF

SEQ ID NO: 93 - protein, 37 aa, Hadronyche versuta
SPTCIPSGQPCPYNENCCSQSCTFKENENGNTVKRCD

SEQ ID NO: 94 - protein, 35 aa, Chilobrachys guangxiensis
ACREWLGGCSKDADCCAHLECRKKWPYHCVWDWTV

SEQ ID NO: 95 - protein, 36 aa, Atrax robustus
SVCIPSGQPCPYNEHCCSGSCTYKENENGNTVQRCD

SEQ ID NO: 96 - protein, 37 aa, Hadronyche versuta
SSTCIPSGQPCPYNENCCSQSCTYKENENGNTVKRCD

SEQ ID NO: 97 - protein, 36 aa, Hadronyche formidabilis
STCTPTDQPCPYHESCCSGSCTYKANENGNQVKRCD

SEQ ID NO: 98 - protein, 37 aa, Hadronyche formidabilis
SPTCIRSGQPCPYNENCCSQSCTFKTNENGNTVKRCD

SEQ ID NO: 99 - protein, 37 aa, Hadronyche infensa
SPTCIPTGQPCPYNENCCSQSCTYKANENGNQVKRCD

SEQ ID NO: 100 - protein, 37 aa, Hadronyche infensa
SSTCIRTDQPCPYNESCCSGSCTYKANENGNQVKRCD

SEQ ID NO: 101 - protein, 37 aa, Hadronyche versuta
SSTCIPSGQPCPYNENCCSQSCTFKENENGNTVKRCD

SEQ ID NO: 102 - protein, 37 aa, Hadronyche versuta
SPTCIPSGQPCPYNENCCSKSCTYKENENGNTVQRCD

SEQ ID NO: 103 - protein, 37 aa, Hadronyche versuta
SPTCIPSGQPCPYNENCCSQSCTYKENENGNTVKRCD

SEQ ID NO: 104 - protein, 38 aa, Macrothele gigas
CMGYDIHCTDRLPCCFGLECVKTSGYWWYKKTYCRRKS

SEQ ID NO: 105 - protein, 39 aa, Missulena bradleyi
SPVCTPSGQPCQPNTQPCCNNAEEEQTINCNGNTVYRCA

SEQ ID NO: 106 - protein, 39 aa, Haplopelma hainanum; identical to SEQ ID NO: 105

SEQ ID NO: 107 - protein, 29 aa, Chilobrachys guangxiensis
YCQKWMWTCDSERKCCEGYVCELWCKYNL

SEQ ID NO: 108 - protein, 33 aa, Chilobrachys guangxiensis
ACGQFWWKCGEGKPPCCANFACKIGLYLCIWSP

SEQ ID NO: 109 - protein, 36 aa, Grammostola rosea
DCVRFWGKCSQTSDCCPHLACKSKWPRNICVWDGSV

SEQ ID NO: 110 - protein, 36 aa, Chilobrachys guangxiensis
DCRALYGGCTKDEDCCKHLACRRTLPTYCAWDLTFP

SEQ ID NO: 111 - protein, 22 aa, Homo sapiens
ILDIVLLFQEHQFEALGLLILL

SEQ ID NO: 112 - protein, 13 aa, Homo sapiens
ILDIVLLFQEHQF

SEQ ID NO: 113 - protein, 38 aa, Ciona intestinalis
VVVVISFGVDIALIFVGESEALAAIGLLVILRLWRVFR

SEQ ID NO: 114 - protein, 36 aa, Homo sapiens
VVVVVSFILDIVLLFQEHQFEALGLLILLRLWRVAR

SEQ ID NO: 115 - protein, 33 aa, synthetic construct
  REGION 1..33: note = toxin sequence element
DCAGYMRECKKDKECCGWNICNRKHKWCKYKLW

SEQ ID NO: 116 - protein, 35 aa, synthetic construct
  REGION 1..35: note = toxin sequence element
GCQMTFWKCNALDHNCCHGYAACGCKKIIVSARIA

SEQ ID NO: 117 - protein, 37 aa, synthetic construct
  REGION 1..37: note = toxin sequence element
GGCLPHNRFCNPSNDQCCKSANLVCRLWCKKKIEGDP

SEQ ID NO: 118 - protein, 39 aa, synthetic construct
  REGION 1..39: note = toxin sequence element
GCKGFGDSCADSDDCCETFHCKWVFFTSKFMCRRVWGKD

SEQ ID NO: 119 - protein, 37 aa, synthetic construct
  REGION 1..37: note = toxin sequence element
GCLGDKCADSDDCCETFHCKWVFFTSKFMCRRVWGKD

SEQ ID NO: 120 - protein, 41 aa, synthetic construct
  REGION 1..41: note = toxin sequence element
SPTCIPSGQPCADSDDCCETFHCKWVFFTSKFMCRRVWGKD

SEQ ID NO: 121 - protein, 38 aa, synthetic construct
  REGION 1..38: note = toxin sequence element
  VARIANT 12: note = Xaa can be any naturally occurring amino acid
  VARIANT 22: note = Xaa can be any naturally occurring amino acid
  VARIANT 36: note = Xaa can be any naturally occurring amino acid
DEDCQPPGNFCXNTSDCCEHLXCPTTPRFPYLCQYXMG

SEQ ID NO: 122 - protein, 29 aa, synthetic construct
  REGION 1..29: note = toxin sequence element
GACRWFLGGCTPEKNDCCQRLYCGPFVCV

SEQ ID NO: 123 - protein, 33 aa, synthetic construct
  REGION 1..33: note = toxin sequence element
SPVCTPSGQPCRENKDCCSKKCKTTGIVKLCRW

SEQ ID NO: 124 - protein, 38 aa, synthetic construct
  REGION 1..38: note = toxin sequence element
ACSKKWEYCTKDSECCPHLGCWKRRRSFEVCVPKTPKT

SEQ ID NO: 125 - protein, 33 aa, synthetic construct
  REGION 1..33: note = toxin sequence element
RCIEEGKWCTKDEDCCKHLACNRKHKWCKYKLW

SEQ ID NO: 126 - protein, 41 aa, synthetic construct
  REGION 1..41: note = toxin sequence element
SPTCIRSGQPCADSDDCCETFHCKWVFFTSKFMCRRVWGKD

SEQ ID NO: 127 - protein, 40 aa, synthetic construct
  REGION 1..40: note = toxin sequence element
STCTPTDQPCADSDDCCETFHCKWVFFTSKFMCRRVWGKD

SEQ ID NO: 128 - protein, 39 aa, synthetic construct
  REGION 1..39: note = toxin sequence element
GCKWYLGDCADSDDCCETFHCKWVFFTSKFMCRRVWGKD

SEQ ID NO: 129 - protein, 41 aa, synthetic construct
  REGION 1..41: note = toxin sequence element
SSTCIPSGQPCADSDDCCETFHCKWVFFTSKFMCRRVWGKD

SEQ ID NO: 130 - protein, 33 aa, synthetic construct
  REGION 1..33: note = toxin sequence element
ACSKKWEYCKEKLCCSGYVCKRRGTNIEKRCRG

SEQ ID NO: 131 - protein, 29 aa, synthetic construct
  REGION 1..29: note = toxin sequence element
ACGQFWWKCTSDSDCCPNWVCRLWCKYKL

SEQ ID NO: 132 - protein, 35 aa, synthetic construct
  REGION 1..35: note = toxin sequence element
CRYWLGGCSQDGDCCKHLQCSPRWGWCIYSTRGGR

SEQ ID NO: 133 - protein, 31 aa, synthetic construct
  REGION 1..31: note = toxin sequence element
DCGTIWHYCTPEKNDCCQRLYCSPRWRLVHL

SEQ ID NO: 134 - protein, 34 aa, synthetic construct
  REGION 1..34: note = toxin sequence element
IACAPRFSICDPKNDKCCPNRVCSDKHKWCKWKL

SEQ ID NO: 135 - protein, 34 aa, synthetic construct
  REGION 1..34: note = toxin sequence element
SSTCIPSGQPCRENKDCCSKKCSDKHKWCKWKLG

SEQ ID NO: 136 - protein, 40 aa, synthetic construct
  REGION 1..40: note = toxin sequence element
DGECGGFWWKCKNSNDCCKDLVCKEKWPITYKICVWDRTF

SEQ ID NO: 137 - protein, 34 aa, synthetic construct
  REGION 1..34: note = toxin sequence element
IACAPRFSLCDTSKDCCEGYVCNRKHKWCKYKLW

SEQ ID NO: 138 - protein, 39 aa, synthetic construct
  REGION 1..39: note = toxin sequence element
ECKGFGKSCADSDDCCETFHCKWVFFTSKFMCRRVWGKD

SEQ ID NO: 139 - protein, 41 aa, synthetic construct
  REGION 1..41: note = toxin sequence element
SPVCTPSGQPCADSDDCCETFHCKWVFFTSKFMCRRVWGKD

SEQ ID NO: 140 - protein, 33 aa, synthetic construct
  REGION 1..33: note = toxin sequence element
DDCGGLFSGCTPGKNECCPNRVCKIGLYLCIWS

SEQ ID NO: 141 - protein, 37 aa, synthetic construct; identical to SEQ ID NO: 119
  REGION 1..37: note = toxin sequence element

SEQ ID NO: 142 - protein, 36 aa, synthetic construct
  REGION 1..36: note = toxin sequence element
CRYLFGGCAWFSGESCCTGICSPRWGWCIYSTRGGR

SEQ ID NO: 143 - protein, 36 aa, synthetic construct
  REGION 1..36: note = toxin sequence element
GDCLPHLKLCNPNDDKCCRPKLKCSRRGTNPEKRCR

SEQ ID NO: 144 - protein, 33 aa, synthetic construct
  REGION 1..33: note = toxin sequence element
DDCGTLFSGCPYSKYCCSGSCKRRGTNIEKRCR

SEQ ID NO: 145 - protein, 29 aa, synthetic construct
  REGION 1..29: note = toxin sequence element
  VARIANT 11: note = Xaa can be any naturally occurring amino acid
AAEGCLCDRCXHSGDCCEDFHCTCEFFNM

SEQ ID NO: 146 - DNA, 99 bp, synthetic construct
  misc_feature 1..99: note = toxin sequence element
  source 1..99: mol_type = other DNA
gattgcgcgggctatatgcgcgaatgtaaaaaagataaagaatgctgcggctggaacatttgcaaccgcaaacataaatggtgcaaatataaactgtgg

SEQ ID NO: 147 - DNA, 105 bp, synthetic construct
  misc_feature 1..105: note = toxin sequence element
  source 1..105: mol_type = other DNA
ggctgccaaatgaccttttggaaatgtaacgcgctggatcacaactgctgccatggctatgccgcctgtggatgcaaaaaaattattgtatccgcgagaatcgcg

SEQ ID NO: 148 - DNA, 111 bp, synthetic construct
  misc_feature 1..111: note = toxin sequence element
  source 1..111: mol_type = other DNA
ggcggctgcctgccgcataaccgcttttgtaacccgagcaacgatcagtgctgcaaaagcgcgaacctggtgtgccgcctgtggtgcaaaaaaaaaattgaaggggatccg

SEQ ID NO: 149 - DNA, 117 bp, synthetic construct
  misc_feature 1..117: note = toxin sequence element
  source 1..117: mol_type = other DNA
ggctgcaaaggctttggcgatagctgtgcggatagcgatgattgctgcgaaacctttcattgcaaatgggtgttttttaccagcaaatttatgtgccgccgcgtgtggggcaaagat

SEQ ID NO: 150 - DNA, 111 bp, synthetic construct
  misc_feature 1..111: note = toxin sequence element
  source 1..111: mol_type = other DNA
ggctgcctgggcgataaatgtgcggatagcgatgattgctgcgaaacctttcattgcaaatgggtgttttttaccagcaaatttatgtgccgccgcgtgtggggcaaagat

SEQ ID NO: 151 - DNA, 127 bp, synthetic construct
  misc_feature 1..127: note = toxin sequence element
  source 1..127: mol_type = other DNA
agcccgacctgcattccgagcggccagccgtgtgcggatagcgatgattgctgcgaaacctttcattgcaaatgggtgttttttaccagcaaatttatgtgccgccgcgtgtggggcaaagatggat

SEQ ID NO: 152 - DNA, 114 bp, synthetic construct
  misc_feature 1..114: note = toxin sequence element
  variation 35: note = n is a, c, g, or t
  variation 64..65: note = n is a, c, g, or t
  variation 107: note = n is a, c, g, or t
  source 1..114: mol_type = other DNA
gacgaagattgccaaccgccgggcaacttttgtancaacaccagcgattgctgcgaacatctgnnctgcccgaccaccccccgctttccctatctgtgccaataccncatggga

SEQ ID NO: 153 - DNA, 88 bp, synthetic construct
  misc_feature 1..88: note = toxin sequence element
  source 1..88: mol_type = other DNA
aggcgcgtgccgctggtttctgggcggctgtaccccggaaaaaaacgattgctgccagcgcctgtattgcggcccgtttgtgtgcgtg

SEQ ID NO: 154 - DNA, 99 bp, synthetic construct
  misc_feature 1..99: note = toxin sequence element
  source 1..99: mol_type = other DNA
agcccggtgtgcaccccgagcggccagccgtgtcgcgaaaacaaagattgctgcagcaaaaaatgcaaaaccaccggcattgtgaaactgtgccgctgg

SEQ ID NO: 155 - DNA, 114 bp, synthetic construct
  misc_feature 1..114: note = toxin sequence element
  source 1..114: mol_type = other DNA
gcgtgcagcaaaaaatgggaatattgtaccaaagatagcgaatgctgcccgcatctgggctgctggaaacgccgccgcagctttgaagtgtgcgtgccgaaaaccccgaaaacc

SEQ ID NO: 156 - DNA, 99 bp, synthetic construct
  misc_feature 1..99: note = toxin sequence element
  source 1..99: mol_type = other DNA
cgctgcattgaagaaggcaaatggtgtaccaaagatgaagattgctgcaaacatctggcgtgcaaccgcaaacataaatggtgcaaatataaactgtgg

SEQ ID NO: 157 - DNA, 123 bp, synthetic construct
  misc_feature 1..123: note = toxin sequence element
  source 1..123: mol_type = other DNA
agcccgacctgcattcgcagcggccagccgtgtgcggatagcgatgattgctgcgaaacctttcattgcaaatgggtgttttttaccagcaaatttatgtgccgccgcgtgtggggcaaagat

SEQ ID NO: 158 - DNA, 120 bp, synthetic construct
  misc_feature 1..120: note = toxin sequence element
  source 1..120: mol_type = other DNA
agcacctgcaccccgaccgatcagccgtgtgcggatagcgatgattgctgcgaaacctttcattgcaaatgggtgttttttaccagcaaatttatgtgccgccgcgtgtggggcaaagat

SEQ ID NO: 159 - DNA, 111 bp, synthetic construct
  misc_feature 1..111: note = toxin sequence element
  source 1..111: mol_type = other DNA
aaatgccgctggctgtttggcggggtaccccgggcaaaaacgaatgctggccgaactatgcgtgccatagctattgggaatggggcctgtgggatggcagctttggatccg

SEQ ID NO: 160 - DNA, 117 bp, synthetic construct
  misc_feature 1..117: note = toxin sequence element
  source 1..117: mol_type = other DNA
ggctgcaaatggtatctgggcgattgtgcggatagcgatgattgctgcgaaacctttcattgcaaatgggtgttttttaccagcaaatttatgtgccgccgcgtgtggggcaaagat

SEQ ID NO: 161 - DNA, 123 bp, synthetic construct
  misc_feature 1..123: note = toxin sequence element
  source 1..123: mol_type = other DNA
agcagcacctgcattccgagcggccagccgtgtgcggatagcgatgattgctgcgaaacctttcattgcaaatgggtgttttttaccagcaaatttatgtgccgccgcgtgtggggcaaagat

SEQ ID NO: 162 - DNA, 99 bp, synthetic construct
  misc_feature 1..99: note = toxin sequence element
  source 1..99: mol_type = other DNA
gcgtgcagcaaaaaatgggaatattgtaaagaaaaactgtgctgcagcggctatgtgtgcaaacgccgcggcaccaacattgaaaaacgctgccgcgga

SEQ ID NO: 163 - DNA, 87 bp, synthetic construct
  misc_feature 1..87: note = toxin sequence element
  source 1..87: mol_type = other DNA
gcgtgcggccagttttggtggaaatgtaccagcgatagcgattgctgcccgaactgggtgtgccgcctgtggtgcaaatataaactg

SEQ ID NO: 164 - DNA, 105 bp, synthetic construct
  misc_feature 1..105: note = toxin sequence element
  source 1..105: mol_type = other DNA
tgccgctattggctgggcggctgtagccaggatggcgattgctgcaaacatctgcagtgcagcccgcgctggggctggtgcatttatagcacccgcggcggccgc

SEQ ID NO: 165 - DNA, 93 bp, synthetic construct
  misc_feature 1..93: note = toxin sequence element
  source 1..93: mol_type = other DNA
gattgcggcaccatttggcattattgtaccccggaaaaaaacgattgctgccagcgcctgtattgcagcccgcgctggaggctggtgcattta

SEQ ID NO: 166 - DNA, 102 bp, synthetic construct
  misc_feature 1..102: note = toxin sequence element
  source 1..102: mol_type = other DNA
attgcgtgcgcgccgcgctttagcatttgtgatccgaaaaacgataaatgctgcccgaaccgcgtgtgcagcgataaacataaatggtgcaaatggaaactg

SEQ ID NO: 167 - DNA, 102 bp, synthetic construct
  misc_feature 1..102: note = toxin sequence element
  source 1..102: mol_type = other DNA
agcagcacctgcattccgagcggccagccgtgtcgcgaaaacaaagattgctgcagcaaaaaatgcagcgataaacataaatggtgcaaatggaaactggga

SEQ ID NO: 168 - DNA, 120 bp, synthetic construct
  misc_feature 1..120: note = toxin sequence element
  source 1..120: mol_type = other DNA
gatggcgaatgcggcggcttttggtggaaatgtaaaaacagcaacgattgctgcaaagatctggtgtgcaaagaaaaatggccgattacctataaaatttgcgtgtgggatcgcaccttt

SEQ ID NO: 169 - DNA, 102 bp, synthetic construct
  misc_feature 1..102: note = toxin sequence element
  source 1..102: mol_type = other DNA
attgcgtgcgcgccgcgctttagcctgtgtgataccagcaaagattgctgcgaaggctatgtgtgcaaccgcaaacataaatggtgcaaatataaactgtgg

SEQ ID NO: 170 - DNA, 117 bp, synthetic construct
  misc_feature 1..117: note = toxin sequence element
  source 1..117: mol_type = other DNA
gaatgcaaaggctttggcaaaagctgtgcggatagcgatgattgctgcgaaacctttcattgcaaatgggtgttttttaccagcaaatttatgtgccgccgcgtgtggggcaaagat

SEQ ID NO: 171 - DNA, 123 bp, synthetic construct
  misc_feature 1..123: note = toxin sequence element
  source 1..123: mol_type = other DNA
agcccggtgtgcaccccgagcggccagccgtgtgcggatagcgatgattgctgcgaaacctttcattgcaaatgggtgttttttaccagcaaatttatgtgccgccgcgtgtggggcaaagat

SEQ ID NO: 172 - DNA, 102 bp, synthetic construct
  misc_feature 1..102: note = toxin sequence element
  source 1..102: mol_type = other DNA
gatgattgcggcggcctgtttagcggctgtaccccgggcaaaaacgaatgctgcccgaaccgcgtgtgcaaaattggcctgtatctgtgcatttggagcccg

SEQ ID NO: 173 - DNA, 111 bp, synthetic construct; identical to SEQ ID NO: 150
  misc_feature 1..111: note = toxin sequence element
  source 1..111: mol_type = other DNA

SEQ ID NO: 174 - DNA, 108 bp, synthetic construct
  misc_feature 1..108: note = toxin sequence element
  source 1..108: mol_type = other DNA
tgccgctatctgtttggcggctgtgcgtggtttagcggcgaaagctgctgcaccggcatttgcagcccgcgctggggctggtgcatttatagcacccgcggcggccgc

SEQ ID NO: 175 - DNA, 108 bp, synthetic construct
  misc_feature 1..108: note = toxin sequence element
  source 1..108: mol_type = other DNA
ggcgattgcctgccgcatctgaaactgtgtaacccgaacgatgataaatgctgccgcccgaaactgaaatgcagccgccgcggcaccaacccggaaaaacgctgccgc

SEQ ID NO: 176 - DNA, 99 bp, synthetic construct
  misc_feature 1..99: note = toxin sequence element
  source 1..99: mol_type = other DNA
gatgattgcggcaccctgtttagcggctgtccgtatagcaaatattgctgcagcggcagctgcaaacgccgcggcaccaacattgaaaaacgctgccgc

SEQ ID NO: 177 - DNA, 86 bp, synthetic construct
  misc_feature 1..86: note = toxin sequence element
  variation 23: note = n is a, c, g, or t
  source 1..86: mol_type = other DNA
gctgcctgtgcgatagatgtgtncatagcggtgattgttgcgaagactttcattgcacctgcgagttttttaacatgtaatttatg

SEQ ID NO: 178 - protein, 10 aa, synthetic construct
  REGION 1..10: note = toxin sequence element
AACLGMFESC

SEQ ID NO: 179 - protein, 9 aa, synthetic construct
  REGION 1..9: note = toxin sequence element
ACGQFWWKC

SEQ ID NO: 180 - protein, 9 aa, synthetic construct
  REGION 1..9: note = toxin sequence element
ACKGVFDAC

SEQ ID NO: 181 - protein, 9 aa, synthetic construct
  REGION 1..9: note = toxin sequence element
ACREWLGGC

SEQ ID NO: 182 - protein, 10 aa, synthetic construct
  REGION 1..10: note = toxin sequence element
ADCGWLFHSC

SEQ ID NO: 183 - protein, 8 aa, synthetic construct
  REGION 1..8: note = toxin sequence element
CAAEGIPC

SEQ ID NO: 184 - protein, 8 aa, synthetic construct
  REGION 1..8: note = toxin sequence element
CGGWMAKC

SEQ ID NO: 185 - protein, 8 aa, synthetic construct
  REGION 1..8: note = toxin sequence element
CIGEGVPC

SEQ ID NO: 186 - protein, 8 aa, synthetic construct
  REGION 1..8: note = toxin sequence element
CMGYDIHC

SEQ ID NO: 187 - protein, 9 aa, synthetic construct
  REGION 1..9: note = toxin sequence element
DCAGYMREC

SEQ ID NO: 188 - protein, 9 aa, synthetic construct
  REGION 1..9: note = toxin sequence element
DCGTIWHYC

SEQ ID NO: 189 - protein, 9 aa, synthetic construct
  REGION 1..9: note = toxin sequence element
DCLGFLWKC

SEQ ID NO: 190 - protein, 9 aa, synthetic construct
  REGION 1..9: note = toxin sequence element
DCLGLFWIC

SEQ ID NO: 191 - protein, 9 aa, synthetic construct
  REGION 1..9: note = toxin sequence element
DCLGWFKGC

SEQ ID NO: 192 - protein, 9 aa, synthetic construct
  REGION 1..9: note = toxin sequence element
DCLGWFKSC

SEQ ID NO: 193 - protein, 9 aa, synthetic construct
  REGION 1..9: note = toxin sequence element
DCRALYGGC

SEQ ID NO: 194 - protein, 9 aa, synthetic construct
  REGION 1..9: note = toxin sequence element
DCRKMFGGC

SEQ ID NO: 195 - protein, 9 aa, synthetic construct
  REGION 1..9: note = toxin sequence element
DCTRMFGAC

SEQ ID NO: 196 - protein, 9 aa, synthetic construct
  REGION 1..9: note = toxin sequence element
DCVRFWGKC

SEQ ID NO: 197 - protein, 10 aa, synthetic construct
  REGION 1..10: note = toxin sequence element
DDCGGLFSGC

SEQ ID NO: 198 - protein, 10 aa, synthetic construct
  REGION 1..10: note = toxin sequence element
DDCGKLFSGC

SEQ ID NO: 199 - protein, 10 aa, synthetic construct
  REGION 1..10: note = toxin sequence element
DDCGTLFSGC

SEQ ID NO: 200 - protein, 10 aa, synthetic construct
  REGION 1..10: note = toxin sequence element
DDCLGMFSSC

SEQ ID NO: 201 - protein, 11 aa, synthetic construct
  REGION 1..11: note = toxin sequence element
DDDCGWIMDDC

SEQ ID NO: 202 - protein, 11 aa, synthetic construct
  REGION 1..11: note = toxin sequence element
DGECGGFWWKC

SEQ ID NO: 203 - protein, 9 aa, synthetic construct
  REGION 1..9: note = toxin sequence element
ECGKFMWKC

SEQ ID NO: 204 - protein, 9 aa, synthetic construct
  REGION 1..9: note = toxin sequence element
ECGTLFSGC

SEQ ID NO: 205 - protein, 9 aa, synthetic construct
  REGION 1..9: note = toxin sequence element
ECKGFGKSC

SEQ ID NO: 206 - protein, 9 aa, synthetic construct
  REGION 1..9: note = toxin sequence element
ECKKLFGGC

SEQ ID NO: 207 - protein, 9 aa, synthetic construct
  REGION 1..9: note = toxin sequence element
ECKWYLGDC

SEQ ID NO: 208 - protein, 9 aa, synthetic construct
  REGION 1..9: note = toxin sequence element
ECKYLWGTC

SEQ ID NO: 209 - protein, 9 aa, synthetic construct
  REGION 1..9: note = toxin sequence element
ECLEIFKAC

SEQ ID NO: 210 - protein, 9 aa, synthetic construct
  REGION 1..9: note = toxin sequence element
ECLGFGKGC

SEQ ID NO: 211 - protein, 9 aa, synthetic construct
  REGION 1..9: note = toxin sequence element
ECRKMFGGC

SEQ ID NO: 212 - protein, 9 aa, synthetic construct
  REGION 1..9: note = toxin sequence element
ECRWLFGGC

SEQ ID NO: 213 - protein, 9 aa, synthetic construct
  REGION 1..9: note = toxin sequence element
ECRWYLGGC

SEQ ID NO: 214 - protein, 9 aa, synthetic construct
  REGION 1..9: note = toxin sequence element
ECRYFWGEC

SEQ ID NO: 215 - protein, 9 aa, synthetic construct
  REGION 1..9: note = toxin sequence element
ECRYLFGGC

SEQ ID NO: 216 - protein, 9 aa, synthetic construct
  REGION 1..9: note = toxin sequence element
ECRYWLGGC

SEQ ID NO: 217 - protein, 9 aa, synthetic construct
  REGION 1..9: note = toxin sequence element
ECRYWLGTC

SEQ ID NO: 218 - protein, 9 aa, synthetic construct
  REGION 1..9: note = toxin sequence element
ECTKFLGGC

SEQ ID NO: 219 - protein, 9 aa, synthetic construct
  REGION 1..9: note = toxin sequence element
ECTKLLGGC

SEQ ID NO: 220 - protein, 10 aa, synthetic construct
  REGION 1..10: note = toxin sequence element
EDCIPKWKGC

SEQ ID NO: 221 - protein, 11 aa, synthetic construct
  REGION 1..11: note = toxin sequence element
EGECGGFWWKC

SEQ ID NO: 222 - protein, 10 aa, synthetic construct
  REGION 1..10: note = toxin sequence element
GACRWFLGGC

SEQ ID NO: 223 - protein, 9 aa, synthetic construct
  REGION 1..9: note = toxin sequence element
GCANAYKSC

SEQ ID NO: 224 - protein, 9 aa, synthetic construct
  REGION 1..9: note = toxin sequence element
GCGGLMAGC

SEQ ID NO: 225 - protein, 9 aa, synthetic construct
  REGION 1..9: note = toxin sequence element
GCGGLMDGC

SEQ ID NO: 226 - protein, 9 aa, synthetic construct
  REGION 1..9: note = toxin sequence element
GCGTMWSPC

SEQ ID NO: 227 - protein, 9 aa, synthetic construct
  REGION 1..9: note = toxin sequence element
GCIPSFGEC

SEQ ID NO: 228 - protein, 9 aa, synthetic construct
  REGION 1..9: note = toxin sequence element
GCKGFGDSC

SEQ ID NO: 229 - protein, 9 aa, synthetic construct
  REGION 1..9: note = toxin sequence element
GCKLTFWKC

SEQ ID NO: 230 - protein, 9 aa, synthetic construct
  REGION 1..9: note = toxin sequence element
GCLEFWWKC

SEQ ID NO: 231 - protein, 7 aa, synthetic construct
  REGION 1..7: note = toxin sequence element
GCLGDKC

SEQ ID NO: 232 - protein, 9 aa, synthetic construct
  REGION 1..9: note = toxin sequence element
GCNRKNKKC

SEQ ID NO: 233 - protein, 9 aa, synthetic construct
  REGION 1..9: note = toxin sequence element
GCQKFFWTC

SEQ ID NO: 234 - protein, 10 aa, synthetic construct
  REGION 1..10: note = toxin sequence element
GDCLPHLKLC

SEQ ID NO: 235 - protein, 10 aa, synthetic construct
  REGION 1..10: note = toxin sequence element
GDCLPHLKRC

SEQ ID NO: 236 - protein, 10 aa, synthetic construct
  REGION 1..10: note = toxin sequence element
GGCLPHNRFC

SEQ ID NO: 237 - protein, 14 aa, synthetic construct
  REGION 1..14: note = toxin sequence element
GVDKAGCRYMFGGC

SEQ ID NO: 238 - protein, 14 aa, synthetic construct
  REGION 1..14: note = toxin sequence element
GVDKEGCRKLLGGC

SEQ ID NO: 239 - protein, 10 aa, synthetic construct
  REGION 1..10: note = toxin sequence element
IACAPRFSIC

SEQ ID NO: 240 - protein, 10 aa, synthetic construct
  REGION 1..10: note = toxin sequence element
IACAPRFSLC

SEQ ID NO: 241 - protein, 10 aa, synthetic construct
  REGION 1..10: note = toxin sequence element
IACAPRGLLC

SEQ ID NO: 242 - protein, 9 aa, synthetic construct
  REGION 1..9: note = toxin sequence element
KCLPPGKPC

SEQ ID NO: 243 - protein, 9 aa, synthetic construct
  REGION 1..9: note = toxin sequence element
LCSREGEFC

SEQ ID NO: 244 - protein, 9 aa, synthetic construct
  REGION 1..9: note = toxin sequence element
QCGEFMWKC

SEQ ID NO: 245 - protein, 9 aa, synthetic construct
  REGION 1..9: note = toxin sequence element
RCIEEGKWC

SEQ ID NO: 246 - protein, 11 aa, synthetic construct
  REGION 1..11: note = toxin sequence element
SAVCIPSGQPC

SEQ ID NO: 247 - protein, 9 aa, synthetic construct
  REGION 1..9: note = toxin sequence element
SCKLTFWRC

SEQ ID NO: 248 - protein, 10 aa, synthetic construct
  REGION 1..10: note = toxin sequence element
SECRWFMGGC

SEQ ID NO: 249 - protein, 12 aa, synthetic construct
  REGION 1..12: note = toxin sequence element
SEKDCIKHLQRC

SEQ ID NO: 250 - protein, 9 aa, synthetic construct
  REGION 1..9: note = toxin sequence element
SFCIPFKPC

SEQ ID NO: 251 - protein, 11 aa, synthetic construct
  REGION 1..11: note = toxin sequence element
SPTCIPSGQPC

SEQ ID NO: 252 - protein, 11 aa, synthetic construct
  REGION 1..11: note = toxin sequence element
SPTCIPTGQPC

SEQ ID NO: 253 - protein, 11 aa, synthetic construct
  REGION 1..11: note = toxin sequence element
SPTCIRSGQPC

SEQ ID NO: 254 - protein, 11 aa, synthetic construct
  REGION 1..11: note = toxin sequence element
SPVCTPSGQPC

SEQ ID NO: 255 - protein, 11 aa, synthetic construct
  REGION 1..11: note = toxin sequence element
SSTCIPSGQPC

SEQ ID NO: 256 - protein, 11 aa, synthetic construct
  REGION 1..11: note = toxin sequence element
SSTCIRTDQPC

SEQ ID NO: 257 - protein, 10 aa, synthetic construct
  REGION 1..10: note = toxin sequence element
STCTPTDQPC

SEQ ID NO: 258 - protein, 10 aa, synthetic construct
  REGION 1..10: note = toxin sequence element
SVCIPSGQPC

SEQ ID NO: 259 - protein, 9 aa, synthetic construct
  REGION 1..9: note = toxin sequence element
TCRYLFGGC

SEQ ID NO: 260 - protein, 9 aa, synthetic construct
  REGION 1..9: note = toxin sequence element
TCYDIGELC

SEQ ID NO: 261 - protein, 9 aa, synthetic construct
  REGION 1..9: note = toxin sequence element
VCRGYGLPC

SEQ ID NO: 262 - protein, 9 aa, synthetic construct
  REGION 1..9: note = toxin sequence element
YCQKWLWTC

SEQ ID NO: 263 - protein, 9 aa, synthetic construct
  REGION 1..9: note = toxin sequence element
YCQKWMWTC

SEQ ID NO: 264 - protein, 8 aa, synthetic construct
  REGION 1..8: note = toxin sequence element
CKQADEPC

SEQ ID NO: 265 - protein, 9 aa, synthetic construct
  REGION 1..9: note = toxin sequence element
ACRKKWEYC

SEQ ID NO: 266 - protein, 11 aa, synthetic construct
  REGION 1..11: note = toxin sequence element
DDDCEPPGNFC

SEQ ID NO: 267 - protein, 11 aa, synthetic construct
  REGION 1..11: note = toxin sequence element
VKPCRKEGQLC

SEQ ID NO: 268 - protein, 9 aa, synthetic construct
  REGION 1..9: note = toxin sequence element
WCKQSGEMC

SEQ ID NO: 269 - protein, 8 aa, synthetic construct
  REGION 1..8: note = toxin sequence element
CLSGGEVC

SEQ ID NO: 270 - protein, 11 aa, synthetic construct
  REGION 1..11: note = toxin sequence element
GKPCHEEGCQL

SEQ ID NO: 271 - protein, 8 aa, synthetic construct
  REGION 1..8: note = toxin sequence element
CIPFLHPC

SEQ ID NO: 272 - protein, 9 aa, synthetic construct
  REGION 1..9: note = toxin sequence element
ACSKKWEYC

SEQ ID NO: 273 - protein, 12 aa, synthetic construct
  REGION 1..12: note = toxin sequence element
ADSDDCCETFHC

SEQ ID NO: 274 - protein, 13 aa, synthetic construct
  REGION 1..13: note = toxin sequence element
AWFSGESCCTGIC

SEQ ID NO: 275 - protein, 12 aa, synthetic construct
  REGION 1..12: note = toxin sequence element
DEERKCCEGLVC

SEQ ID NO: 276 - protein, 13 aa, synthetic construct
  REGION 1..13: note = toxin sequence element
DENDPRCCSGLVC

SEQ ID NO: 277 - protein, 13 aa, synthetic construct
  REGION 1..13: note = toxin sequence element
DGKSTFCCSGFNC

SEQ ID NO: 278 - protein, 13 aa, synthetic construct
  REGION 1..13: note = toxin sequence element
DGKSTFCCSGYNC

SEQ ID NO: 279 - protein, 13 aa, synthetic construct
  REGION 1..13: note = toxin sequence element
DPDNDKCCEGYKC

SEQ ID NO: 280 - protein, 13 aa, synthetic construct
  REGION 1..13: note = toxin sequence element
DPKNDKCCKNYTC

SEQ ID NO: 281 - protein, 13 aa, synthetic construct
  REGION 1..13: note = toxin sequence element
DPKNDKCCPNRVC

SEQ ID NO: 282 - protein, 13 aa, synthetic construct
  REGION 1..13: note = toxin sequence element
DPNNDKCCPNREC

SEQ ID NO: 283 - protein, 13 aa, synthetic construct
  REGION 1..13: note = toxin sequence element
DPNNDKCCPNRVC

SEQ ID NO: 284 - protein, 16 aa, synthetic construct
  REGION 1..16: note = toxin sequence element
DPNPVKDLPCCSGLAC

SEQ ID NO: 285 - protein, 12 aa, synthetic construct
  REGION 1..12: note = toxin sequence element
DSARKCCEGLVC

SEQ ID NO: 286 - protein, 12 aa, synthetic construct
  REGION 1..12: note = toxin sequence element
DSERKCCEDMVC

SEQ ID NO: 287 - protein, 12 aa, synthetic construct
  REGION 1..12: note = toxin sequence element
DSERKCCEGMVC

SEQ ID NO: 288 - protein, 12 aa, synthetic construct
  REGION 1..12: note = toxin sequence element
DSERKCCEGYVC

SEQ ID NO: 289 - protein, 12 aa, synthetic construct
  REGION 1..12: note = toxin sequence element
DSKRACCEGLRC

SEQ ID NO: 290 - protein, 12 aa, synthetic construct
  REGION 1..12: note = toxin sequence element
DSKRKCCEDMVC

SEQ ID NO: 291 - protein, 12 aa, synthetic construct
  REGION 1..12: note = toxin sequence element
DSNADCCEGYVC

SEQ ID NO: 292 - protein, 12 aa, synthetic construct
  REGION 1..12: note = toxin sequence element
DSTLDCCKHLSC

SEQ ID NO: 293 - protein, 12 aa, synthetic construct
  REGION 1..12: note = toxin sequence element
DTNADCCEGYVC

SEQ ID NO: 294 - protein, 12 aa, synthetic construct
  REGION 1..12: note = toxin sequence element
DTSKDCCEGYVC

SEQ ID NO: 295 - protein, 12 aa, synthetic construct
  REGION 1..12: note = toxin sequence element
DYNNGCCSGYVC

SEQ ID NO: 296 - protein, 12 aa, synthetic construct
  REGION 1..12: note = toxin sequence element
EKDEHCCEHLGC

SEQ ID NO: 297 - protein, 12 aa, synthetic construct
  REGION 1..12: note = toxin sequence element
EKDSDCCEHLGC

SEQ ID NO: 298 - protein, 12 aa, synthetic construct
  REGION 1..12: note = toxin sequence element
ESNADCCENWAC

SEQ ID NO: 299 - protein, 12 aa, synthetic construct
  REGION 1..12: note = toxin sequence element
FRDKECCKGLTC

SEQ ID NO: 300 - protein, 13 aa, synthetic construct
  REGION 1..13: note = toxin sequence element
GAGKPTCCSGYDC

SEQ ID NO: 301 - protein, 13 aa, synthetic construct
  REGION 1..13: note = toxin sequence element
GEGKPPCCANFAC

SEQ ID NO: 302 - protein, 13 aa, synthetic construct
  REGION 1..13: note = toxin sequence element
GRGKPPCCKGYAC

SEQ ID NO: 303 - protein, 13 aa, synthetic construct
  REGION 1..13: note = toxin sequence element
GSGKPACCPKYVC

SEQ ID NO: 304 - protein, 13 aa, synthetic construct
  REGION 1..13: note = toxin sequence element
GTDQSECCEGWKC

SEQ ID NO: 305 - protein, 13 aa, synthetic construct
  REGION 1..13: note = toxin sequence element
HPGQPPCCSGLAC

SEQ ID NO: 306 - protein, 11 aa, synthetic construct
  REGION 1..11: note = toxin sequence element
KADNDCCGKKC

SEQ ID NO: 307 - protein, 12 aa, synthetic construct
  REGION 1..12: note = toxin sequence element
KAHEDCCEHLRC

SEQ ID NO: 308 - protein, 12 aa, synthetic construct
  REGION 1..12: note = toxin sequence element
KEDSECCEHLQC

SEQ ID NO: 309 - protein, 11 aa, synthetic construct
  REGION 1..11: note = toxin sequence element
KEKLCCSGYVC

SEQ ID NO: 310 - protein, 11 aa, synthetic construct
  REGION 1..11: note = toxin sequence element
KENKDCCSKKC

SEQ ID NO: 311 - protein, 12 aa, synthetic construct
  REGION 1..12: note = toxin sequence element
KKDKECCGWNIC

SEQ ID NO: 312 - protein, 12 aa, synthetic construct
  REGION 1..12: note = toxin sequence element
KNKKECCGWNAC

SEQ ID NO: 313 - protein, 12 aa, synthetic construct
  REGION 1..12: note = toxin sequence element
KNSNDCCKDLVC

SEQ ID NO: 314 - protein, 12 aa, synthetic construct
  REGION 1..12: note = toxin sequence element
KSDENCCKKFKC

SEQ ID NO: 315 - protein, 12 aa, synthetic construct
  REGION 1..12: note = toxin sequence element
KSTSDCCEHLSC

SEQ ID NO: 316 - protein, 12 aa, synthetic construct
  REGION 1..12: note = toxin sequence element
KTTADCCKHLAC

SEQ ID NO: 317 - protein, 12 aa, synthetic construct
  REGION 1..12: note = toxin sequence element
KTTADCCKHLGC

SEQ ID NO: 318 - protein, 12 aa, synthetic construct
  REGION 1..12: note = toxin sequence element
KTTSDCCKHLGC

SEQ ID NO: 319 - protein, 14 aa, synthetic construct
  REGION 1..14: note = toxin sequence element
NALSGPRCCSGLKC

SEQ ID NO: 320 - protein, 12 aa, synthetic construct
  REGION 1..12: note = toxin sequence element
NDEMVCCEHLVC

SEQ ID NO: 321 - protein, 17 aa, synthetic construct
  REGION 1..17: note = toxin sequence element
NGPHTCCWGYNGYKKAC

SEQ ID NO: 322 - protein, 13 aa, synthetic construct
  REGION 1..13: note = toxin sequence element
NPDNDKCCEGRKC

SEQ ID NO: 323 - protein, 14 aa, synthetic construct
  REGION 1..14: note = toxin sequence element
NPNDDKCCRPKLKC

SEQ ID NO: 324 - protein, 14 aa, synthetic construct
  REGION 1..14: note = toxin sequence element
NPSNDKCCRPNLVC

SEQ ID NO: 325 - protein, 15 aa, synthetic construct
  REGION 1..15: note = toxin sequence element
NPSNDQCCKSANLVC

SEQ ID NO: 326 - protein, 15 aa, synthetic construct
  REGION 1..15: note = toxin sequence element
NPSNDQCCKSSKLVC

SEQ ID NO: 327 - protein, 15 aa, synthetic construct
  REGION 1..15: note = toxin sequence element
NPSNDQCCKSSNLVC

SEQ ID NO: 328 - protein, 13 aa, synthetic construct
  REGION 1..13: note = toxin sequence element
NSDADCCRYGERC

SEQ ID NO: 329 - protein, 12 aa, synthetic construct
  REGION 1..12: note = toxin sequence element
NSDKECCKGLRC

SEQ ID NO: 330 - protein, 13 aa, synthetic construct
  REGION 1..13: note = toxin sequence element
NYMDDKCCPGYKC

SEQ ID NO: 331 - protein, 12 aa, synthetic construct
  REGION 1..12: note = toxin sequence element
PKKAPCCGRLEC

SEQ ID NO: 332 - protein, 11 aa, synthetic construct
  REGION 1..11: note = toxin sequence element
PYHESCCSGSC

SEQ ID NO: 333 - protein, 11 aa, synthetic construct
  REGION 1..11: note = toxin sequence element
PYNEHCCSGSC

SEQ ID NO: 334 - protein, 11 aa, synthetic construct
  REGION 1..11: note = toxin sequence element
PYNENCCSKSC

SEQ ID NO: 335 - protein, 11 aa, synthetic construct
  REGION 1..11: note = toxin sequence element
PYNENCCSQSC

SEQ ID NO: 336 - protein, 11 aa, synthetic construct
  REGION 1..11: note = toxin sequence element
PYNESCCSGSC

SEQ ID NO: 337 - protein, 11 aa, synthetic construct
  REGION 1..11: note = toxin sequence element
PYSKYCCSGSC

SEQ ID NO: 338 - protein, 19 aa, synthetic construct
  REGION 1..19: note = toxin sequence element
QPNTQPCCNNAEEEQTINC

SEQ ID NO: 339 - protein, 11 aa, synthetic construct
  REGION 1..11: note = toxin sequence element
RENKDCCSKKC

SEQ ID NO: 340 - protein, 12 aa, synthetic construct
  REGION 1..12: note = toxin sequence element
RRDSDCCPHLGC

SEQ ID NO: 341 - protein, 12 aa, synthetic construct
  REGION 1..12: note = toxin sequence element
SAGQTCCKHLVC

SEQ ID NO: 342 - protein, 12 aa, synthetic construct
  REGION 1..12: note = toxin sequence element
SEDSECCPHLGC

SEQ ID NO: 343 - protein, 12 aa, synthetic construct
  REGION 1..12: note = toxin sequence element
SKDADCCAHLEC

SEQ ID NO: 344 - protein, 12 aa, synthetic construct
  REGION 1..12: note = toxin sequence element
SKHEDCCAHLAC

SEQ ID NO: 345 - protein, 12 aa, synthetic construct
  REGION 1..12: note = toxin sequence element
SKTGDCCSHLSC

SEQ ID NO: 346 - protein, 12 aa, synthetic construct
  REGION 1..12: note = toxin sequence element
SQDGDCCKHLQC

SEQ ID NO: 347 - protein, 12 aa, synthetic construct
  REGION 1..12: note = toxin sequence element
SQTSDCCPHLAC

SEQ ID NO: 348 - protein, 12 aa, synthetic construct
  REGION 1..12: note = toxin sequence element
SSDKPCCSGYYC

SEQ ID NO: 349 - protein, 12 aa, synthetic construct
  REGION 1..12: note = toxin sequence element
SSTSDCCKHLSC

SEQ ID NO: 350 - protein, 12 aa, synthetic construct
  REGION 1..12: note = toxin sequence element
STEKPCCDNFSC

SEQ ID NO: 351 - protein, 12 aa, synthetic construct
  REGION 1..12: note = toxin sequence element
STHADCCEGFIC

SEQ ID NO: 352 - protein, 12 aa, synthetic construct
  REGION 1..12: note = toxin sequence element
SVDSDCCAHLGC

SEQ ID NO: 353 - protein, 12 aa, synthetic construct
  REGION 1..12: note = toxin sequence element
SVHSDCCAHLGC

SEQ ID NO: 354 - protein, 12 aa, synthetic construct
  REGION 1..12: note = toxin sequence element
SVNDDCCPRLGC

SEQ ID NO: 355 - protein, 12 aa, synthetic construct
  REGION 1..12: note = toxin sequence element
TDRLPCCFGLEC

SEQ ID NO: 356 - protein, 12 aa, synthetic construct
  REGION 1..12: note = toxin sequence element
TIDDDCCPHLGC

SEQ ID NO: 357 - protein, 12 aa, synthetic construct
  REGION 1..12: note = toxin sequence element
TKDEDCCKHLAC

SEQ ID NO: 358 - protein, 12 aa, synthetic construct
  REGION 1..12: note = toxin sequence element
TKDSECCPHLGC

SEQ ID NO: 359 - protein, 13 aa, synthetic construct
  REGION 1..13: note = toxin sequence element
TPEKNDCCQRLYC

SEQ ID NO: 360 - protein, 13 aa, synthetic construct
  REGION 1..13: note = toxin sequence element
TPGKNECCPNRVC

SEQ ID NO: 361 - protein, 13 aa, synthetic construct
  REGION 1..13: note = toxin sequence element
TPGKNECCPNYAC

SEQ ID NO: 362 - protein, 12 aa, synthetic construct
  REGION 1..12: note = toxin sequence element
TSDSDCCPNWVC

SEQ ID NO: 363 - protein, 12 aa, synthetic construct
  REGION 1..12: note = toxin sequence element
TTSSECCAHLGC

SEQ ID NO: 364 - protein, 13 aa, synthetic construct
  REGION 1..13: note = toxin sequence element
VNRHGDCCEGLEC

SEQ ID NO: 365 - protein, 13 aa, synthetic construct
  REGION 1..13: note = toxin sequence element
VPGKNECCSGYAC

SEQ ID NO: 366 - protein, 13 aa, synthetic construct
  REGION 1..13: note = toxin sequence element
YGATQKIPCCGVC

SEQ ID NO: 367 - protein, 12 aa, synthetic construct
  REGION 1..12: note = toxin sequence element
YKLRKCCAGFYC

SEQ ID NO: 368 - protein, 12 aa, synthetic construct
  REGION 1..12: note = toxin sequence element
DVFSLDCCTGIC

SEQ ID NO: 369 - protein, 16 aa, synthetic construct
  REGION 1..16: note = toxin sequence element
IVPIIGFIYCCPGLIC

SEQ ID NO: 370 - protein, 14 aa, synthetic construct
  REGION 1..14: note = toxin sequence element
GMIKIGPPCCSGWC

SEQ ID NO: 371 - protein, 13 aa, synthetic construct
  REGION 1..13: note = toxin sequence element
DPIFQNCCRGWNC

SEQ ID NO: 372 - protein, 12 aa, synthetic construct
  REGION 1..12: note = toxin sequence element
NVLDQNCCDGYC

SEQ ID NO: 373 - protein, 11 aa, synthetic construct
  REGION 1..11: note = toxin sequence element
DFLFPKCCNYC

SEQ ID NO: 374 - protein, 13 aa, synthetic construct
  REGION 1..13: note = toxin sequence element
DPFLQNCCLGWNC

SEQ ID NO: 375 - protein, 12 aa, synthetic construct
  REGION 1..12: note = toxin sequence element
TFFFPDCCNSIC

SEQ ID NO: 376 - protein, 16 aa, synthetic construct
  REGION 1..16: note = toxin sequence element
IVPILGFVYCCPGLIC

SEQ ID NO: 377 - protein, 8 aa, synthetic construct
  REGION 1..8: note = toxin sequence element
ALGICMPR

SEQ ID NO: 378 - protein, 14 aa, synthetic construct
  REGION 1..14: note = toxin sequence element
ATTGRFRYLCKYQI

SEQ ID NO: 379 - protein, 12 aa, synthetic construct
  REGION 1..12: note = toxin sequence element
DRRDQWCKWNPW

SEQ ID NO: 380 - protein, 8 aa, synthetic construct
  REGION 1..8: note = toxin sequence element
ELWCKYNL

SEQ ID NO: 381 - protein, 12 aa, synthetic construct
  REGION 1..12: note = toxin sequence element
ERSSPWCKIDIW

SEQ ID NO: 382 - protein, 7 aa, synthetic construct
  REGION 1..7: note = toxin sequence element
HLWCKYK

SEQ ID NO: 383 - protein, 15 aa, synthetic construct
  REGION 1..15: note = toxin sequence element
HSLFSYCAWDLTFSD

SEQ ID NO: 384 - protein, 15 aa, synthetic construct
  REGION 1..15: note = toxin sequence element
HSNYEWCIWDGTFSK

SEQ ID NO: 385 - protein, 12 aa, synthetic construct
  REGION 1..12: note = toxin sequence element
HSNYEWCVWDGT

SEQ ID NO: 386 - protein, 13 aa, synthetic construct
  REGION 1..13: note = toxin sequence element
HSRWDWCIWDGTF

SEQ ID NO: 387 - protein, 13 aa, synthetic construct
  REGION 1..13: note = toxin sequence element
HSYWEWCLWDGSF

SEQ ID NO: 388 - protein, 8 aa, synthetic construct
  REGION 1..8: note = toxin sequence element
  VARIANT 5: note = Xaa can be any naturally occurring amino acid
ICSGXNWK

SEQ ID NO: 389 - protein, 15 aa, synthetic construct
  REGION 1..15: note = toxin sequence element
ISTKVNYYCRPDRGP

SEQ ID NO: 390 - protein, 10 aa, synthetic construct
  REGION 1..10: note = toxin sequence element
KAFVLHCYRN

SEQ ID NO: 391 - protein, 13 aa, synthetic construct
  REGION 1..13: note = toxin sequence element
KDVLYYCAWDGTF

SEQ ID NO: 392 - protein, 17 aa, synthetic construct
  REGION 1..17: note = toxin sequence element
KEKWPITYKICVWDRTF

SEQ ID NO: 393 - protein, 11 aa, synthetic construct
  REGION 1..11: note = toxin sequence element
KELSIWDSRCL

SEQ ID NO: 394 - protein, 14 aa, synthetic construct
  REGION 1..14: note = toxin sequence element
KFRDKYCAWDFTFS

SEQ ID NO: 395 - protein, 13 aa, synthetic construct
  REGION 1..13: note = toxin sequence element
KGPSPKQKKCTRP

SEQ ID NO: 396 - protein, 14 aa, synthetic construct
  REGION 1..14: note = toxin sequence element
KGRFVNTWPTFCLV

SEQ ID NO: 397 - protein, 11 aa, synthetic construct
  REGION 1..11: note = toxin sequence element
KIGLYLCIWSP

SEQ ID NO: 398 - protein, 9 aa, synthetic construct
  REGION 1..9: note = toxin sequence element
KLWCRKIIG

SEQ ID NO: 399 - protein, 10 aa, synthetic construct
  REGION 1..10: note = toxin sequence element
KLWCRYERTW

SEQ ID NO: 400 - protein, 13 aa, synthetic construct
  REGION 1..13: note = toxin sequence element
KMGLDYCAWDGTF

SEQ ID NO: 401 - protein, 13 aa, synthetic construct
  REGION 1..13: note = toxin sequence element
KMGLYYCAWDGTF

SEQ ID NO: 402 - protein, 12 aa, synthetic construct
  REGION 1..12: note = toxin sequence element
KPTLKYCAWDGT

SEQ ID NO: 403 - protein, 13 aa, synthetic construct
  REGION 1..13: note = toxin sequence element
KPTLKYCAWDGTF

SEQ ID NO: 404 - protein, 13 aa, synthetic construct
  REGION 1..13: note = toxin sequence element
KPTSKYCAWDGTI

SEQ ID NO: 405 - protein, 14 aa, synthetic construct
  REGION 1..14: note = toxin sequence element
KQKWPFYCAWDWSF

SEQ ID NO: 406 - protein, 12 aa, synthetic construct
  REGION 1..12: note = toxin sequence element
KRRGTNAEKRCR

SEQ ID NO: 407 - protein, 12 aa, synthetic construct
  REGION 1..12: note = toxin sequence element
KRRGTNIEKRCR

SEQ ID NO: 408 - protein, 15 aa, synthetic construct
  REGION 1..15: note = toxin sequence element
KRTFNYCAWDGSFSK

SEQ ID NO: 409 - protein, 15 aa, synthetic construct
  REGION 1..15: note = toxin sequence element
KSKWPRNICVWDGSV

SEQ ID NO: 410 - protein, 11 aa, synthetic construct
  REGION 1..11: note = toxin sequence element
KTTGIVKLCRW

SEQ ID NO: 411 - protein, 18 aa, synthetic construct
  REGION 1..18: note = toxin sequence element
KWVFFTSKFMCRRVWGKD

SEQ ID NO: 412 - protein, 18 aa, synthetic construct
  REGION 1..18: note = toxin sequence element
LKPTLHGIWYKHHYCYTQ

SEQ ID NO: 413 - protein, 18 aa, synthetic construct
  REGION 1..18: note = toxin sequence element
LKPTLHGIWYKSYYCYKK

SEQ ID NO: 414 - protein, 9 aa, synthetic construct
  REGION 1..9: note = toxin sequence element
NGNTVYRCA

SEQ ID NO: 415 - protein, 13 aa, synthetic construct
  REGION 1..13: note = toxin sequence element
NKKHGWCGWDGTF

SEQ ID NO: 416 - protein, 13 aa, synthetic construct
  REGION 1..13: note = toxin sequence element
NKKYWHCGWDGTF

SEQ ID NO: 417 - protein, 12 aa, synthetic construct
  REGION 1..12: note = toxin sequence element
NRKHKWCKYKLW

SEQ ID NO: 418 - protein, 12 aa, synthetic construct
  REGION 1..12: note = toxin sequence element
NRRDKWCKYKLW

SEQ ID NO: 419 - protein, 11 aa, synthetic construct
  REGION 1..11: note = toxin sequence element
NSRDKWCKVLL

SEQ ID NO: 420 - protein, 8 aa, synthetic construct
  REGION 1..8: note = toxin sequence element
QLWCKKRL

SEQ ID NO: 421 - protein, 11 aa, synthetic construct
  REGION 1..11: note = toxin sequence element
QPAIKWCIWSP

SEQ ID NO: 422 - protein, 14 aa, synthetic construct
  REGION 1..14: note = toxin sequence element
QSRIANMWPTFCLV

SEQ ID NO: 423 - protein, 14 aa, synthetic construct
  REGION 1..14: note = toxin sequence element
QSRIANMWPTFCSQ

SEQ ID NO: 424 - protein, 14 aa, synthetic construct
  REGION 1..14: note = toxin sequence element
RKKWPYHCGWDGTF

SEQ ID NO: 425 - protein, 14 aa, synthetic construct
  REGION 1..14: note = toxin sequence element
RKKWPYHCVWDWTV

SEQ ID NO: 426 - protein, 8 aa, synthetic construct
  REGION 1..8: note = toxin sequence element
RLWCKKII

SEQ ID NO: 427 - protein, 11 aa, synthetic construct
  REGION 1..11: note = toxin sequence element
RLWCKKKIEEG

SEQ ID NO: 428 - protein, 10 aa, synthetic construct
  REGION 1..10: note = toxin sequence element
RLWCKKKIEW

SEQ ID NO: 429 - protein, 9 aa, synthetic construct
  REGION 1..9: note = toxin sequence element
RLWCKKKLW

SEQ ID NO: 430 - protein, 8 aa, synthetic construct
  REGION 1..8: note = toxin sequence element
RLWCKKRL

SEQ ID NO: 431 - protein, 8 aa, synthetic construct
  REGION 1..8: note = toxin sequence element
RLWCKLDW

SEQ ID NO: 432 - protein, 10 aa, synthetic construct
  REGION 1..10: note = toxin sequence element
RLWCKRIINM

SEQ ID NO: 433 - protein, 8 aa, synthetic construct
  REGION 1..8: note = toxin sequence element
RLWCKYKL

SEQ ID NO: 434 - protein, 14 aa, synthetic construct
  REGION 1..14: note = toxin sequence element
RRAKPSWCGWDFTF

SEQ ID NO: 435 - protein, 14 aa, synthetic construct
  REGION 1..14: note = toxin sequence element
RRAKPSWCGWDFTV

SEQ ID NO: 436 - protein, 15 aa, synthetic construct
  REGION 1..15: note = toxin sequence element
RRTLPTYCAWDLTFP

SEQ ID NO: 437 - protein, 13 aa, synthetic construct
  REGION 1..13: note = toxin sequence element
RSDGKYCAWDGTF

SEQ ID NO: 438 - protein, 14 aa, synthetic construct
  REGION 1..14: note = toxin sequence element
RSDWKYCAWDGTFS

SEQ ID NO: 439 - protein, 12 aa, synthetic construct
  REGION 1..12: note = toxin sequence element
RSRDQWCKYKLW

SEQ ID NO: 440 - protein, 12 aa, synthetic construct
  REGION 1..12: note = toxin sequence element
RVRDQWCKYKLW

SEQ ID NO: 441 - protein, 11 aa, synthetic construct
  REGION 1..11: note = toxin sequence element
SDKHKWCKWKL

SEQ ID NO: 442 - protein, 6 aa, synthetic construct
  REGION 1..6: note = toxin sequence element
SHNKCT

SEQ ID NO: 443 - protein, 11 aa, synthetic construct
  REGION 1..11: note = toxin sequence element
SKLFKLCNFSF

SEQ ID NO: 444 - protein, 14 aa, synthetic construct
  REGION 1..14: note = toxin sequence element
SKTGFVKNICKYEM

SEQ ID NO: 445 - protein, 12 aa, synthetic construct
  REGION 1..12: note = toxin sequence element
SKTWGWCAVEAP

SEQ ID NO: 446 - protein, 15 aa, synthetic construct
  REGION 1..15: note = toxin sequence element
SPKHGWCVWDWTFRK

SEQ ID NO: 447 - protein, 12 aa, synthetic construct
  REGION 1..12: note = toxin sequence element
SPKWGLCNFPMP

SEQ ID NO: 448 - protein, 15 aa, synthetic construct
  REGION 1..15: note = toxin sequence element
SPRWGWCIYSTRGGR

SEQ ID NO: 449 - protein, 15 aa, synthetic construct
  REGION 1..15: note = toxin sequence element
SPTWKWCVLKSPGRR

SEQ ID NO: 450 - protein, 12 aa, synthetic construct
  REGION 1..12: note = toxin sequence element
SPTWKWCVYARP

SEQ ID NO: 451 - protein, 10 aa, synthetic construct
  REGION 1..10: note = toxin sequence element
SQHRLCSVKA

SEQ ID NO: 452 - protein, 11 aa, synthetic construct
  REGION 1..11: note = toxin sequence element
SRKDKWCKYQI

SEQ ID NO: 453 - protein, 11 aa, synthetic construct
  REGION 1..11: note = toxin sequence element
SRKHRWCKYEI

SEQ ID NO: 454 - protein, 11 aa, synthetic construct
  REGION 1..11: note = toxin sequence element
SRKTRWCKYQI

SEQ ID NO: 455 - protein, 11 aa, synthetic construct
  REGION 1..11: note = toxin sequence element
SRQLCKYVIDW

SEQ ID NO: 456 - protein, 11 aa, synthetic construct
  REGION 1..11: note = toxin sequence element
SRRDRWCKYDL

SEQ ID NO: 457 - protein, 11 aa, synthetic construct
  REGION 1..11: note = toxin sequence element
SRRDRWCKYYL

SEQ ID NO: 458 - protein, 12 aa, synthetic construct
  REGION 1..12: note = toxin sequence element
SRRGTNPEKRCR

SEQ ID NO: 459 - protein, 14 aa, synthetic construct
  REGION 1..14: note = toxin sequence element
SRRHGWCVWDGTFS

SEQ ID NO: 460 - protein, 13 aa, synthetic construct
  REGION 1..13: note = toxin sequence element
SRTWKWCVLAGPW

SEQ ID NO: 461 - protein, 11 aa, synthetic construct
  REGION 1..11: note = toxin sequence element
SSKHKWCKVYL

SEQ ID NO: 462 - protein, 13 aa, synthetic construct
  REGION 1..13: note = toxin sequence element
SSRWKWCVLASPF

SEQ ID NO: 463 - protein, 13 aa, synthetic construct
  REGION 1..13: note = toxin sequence element
SSRWKWCVLPAPW

SEQ ID NO: 464 - protein, 15 aa, synthetic construct
  REGION 1..15: note = toxin sequence element
TFKENENGNTVKRCD

SEQ ID NO: 465 - protein, 15 aa, synthetic construct
  REGION 1..15: note = toxin sequence element
TFKTNENGNTVKRCD

SEQ ID NO: 466 - protein, 7 aa, synthetic construct
  REGION 1..7: note = toxin sequence element
TGLCIPP

SEQ ID NO: 467 - protein, 8 aa, synthetic construct
  REGION 1..8: note = toxin sequence element
TRFNVCGK

SEQ ID NO: 468 - protein, 9 aa, synthetic construct
  REGION 1..9: note = toxin sequence element
TWPTEICID

SEQ ID NO: 469 - protein, 15 aa, synthetic construct
  REGION 1..15: note = toxin sequence element
TYKANENGNQVKRCD

SEQ ID NO: 470 - protein, 15 aa, synthetic construct
  REGION 1..15: note = toxin sequence element
TYKENENGNTVKRCD

SEQ ID NO: 471 - protein, 15 aa, synthetic construct
  REGION 1..15: note = toxin sequence element
TYKENENGNTVQRCD

SEQ ID NO: 472 - protein, 15 aa, synthetic construct
  REGION 1..15: note = toxin sequence element
TYKTNENGNSVQRCD

SEQ ID NO: 473 - protein, 18 aa, synthetic construct
  REGION 1..18: note = toxin sequence element
VKTSGYWWYKKTYCRRKS

SEQ ID NO: 474 - protein, 17 aa, synthetic construct
  REGION 1..17: note = toxin sequence element
WKRRRSFEVCVPKTPKT

SEQ ID NO: 475 - protein, 6 aa, synthetic construct
  REGION 1..6: note = toxin sequence element
LGVCMW

SEQ ID NO: 476 - protein, 5 aa, synthetic construct
  REGION 1..5: note = toxin sequence element
FFACA

SEQ ID NO: 477 - protein, 5 aa, synthetic construct
  REGION 1..5: note = toxin sequence element
VLFCV

SEQ ID NO: 478 - protein, 6 aa, synthetic construct
  REGION 1..6: note = toxin sequence element
IVFVCT

SEQ ID NO: 479 - protein, 6 aa, synthetic construct
  REGION 1..6: note = toxin sequence element
ILLFCS

SEQ ID NO: 480 - protein, 5 aa, synthetic construct
  REGION 1..5: note = toxin sequence element
VFVCI

SEQ ID NO: 481 - protein, 6 aa, synthetic construct
  REGION 1..6: note = toxin sequence element
AQFICL

SEQ ID NO: 482 - protein, 6 aa, synthetic construct
  REGION 1..6: note = toxin sequence element
GPFVCV

SEQ ID NO: 483 - DNA, 30 bp, synthetic construct
  misc_feature 1..30: note = toxin sequence element
  source 1..30: mol_type = other DNA
gcggcgtgcctgggcatgtttgaaagctgt

SEQ ID NO: 484 - DNA, 27 bp, synthetic construct
  misc_feature 1..27: note = toxin sequence element
  source 1..27: mol_type = other DNA
gcgtgcggccagttttggtggaaatgt

SEQ ID NO: 485 - DNA, 27 bp, synthetic construct
  misc_feature 1..27: note = toxin sequence element
  source 1..27: mol_type = other DNA
gcgtgcaaaggcgtgtttgatgcgtgt

SEQ ID NO: 486 - DNA, 27 bp, synthetic construct
  misc_feature 1..27: note = toxin sequence element
  source 1..27: mol_type = other DNA
gcgtgccgcgaatggctgggcggctgt

SEQ ID NO: 487 - DNA, 30 bp, synthetic construct
  misc_feature 1..30: note = toxin sequence element
  source 1..30: mol_type = other DNA
gcggattgcggctggctgtttcatagctgt

SEQ ID NO: 488 - DNA, 24 bp, synthetic construct
  misc_feature 1..24: note = toxin sequence element
  source 1..24: mol_type = other DNA
tgcgcggcggaaggcattccgtgt

SEQ ID NO: 489 - DNA, 24 bp, synthetic construct
  misc_feature 1..24: note = toxin sequence element
  source 1..24: mol_type = other DNA
tgcggcggctggatggcgaaatgt

SEQ ID NO: 490 - DNA, 24 bp, synthetic construct
  misc_feature 1..24: note = toxin sequence element
  source 1..24: mol_type = other DNA
tgcattggcgaaggcgtgccgtgt

SEQ ID NO: 491 - DNA, 24 bp, synthetic construct
  misc_feature 1..24: note = toxin sequence element
  source 1..24: mol_type = other DNA
tgcatgggctatgatattcattgt

SEQ ID NO: 492 - DNA, 27 bp, synthetic construct
  misc_feature 1..27: note = toxin sequence element
  source 1..27: mol_type = other DNA
gattgcgcgggctatatgcgcgaatgt

SEQ ID NO: 493 - DNA, 27 bp, synthetic construct
  misc_feature 1..27: note = toxin sequence element
  source 1..27: mol_type = other DNA
gattgcggcaccatttggcattattgt

SEQ ID NO: 494 - DNA, 27 bp, synthetic construct
  misc_feature 1..27: note = toxin sequence element
  source 1..27: mol_type = other DNA
gattgcctgggctttctgtggaaatgt

SEQ ID NO: 495 - DNA, 26 bp, synthetic construct
  misc_feature 1..26: note = toxin sequence element
  source 1..26: mol_type = other DNA
gattgcctgggcctgttttggattgt

SEQ ID NO: 496 - DNA, 27 bp, synthetic construct
  misc_feature 1..27: note = toxin sequence element
  source 1..27: mol_type = other DNA
gattgcctgggctggtttaaaggctgt

SEQ ID NO: 497 - DNA, 27 bp, synthetic construct
  misc_feature 1..27: note = toxin sequence element
  source 1..27: mol_type = other DNA
gattgcctgggctggtttaaaagctgt

SEQ ID NO: 498 - DNA, 27 bp, synthetic construct
  misc_feature 1..27: note = toxin sequence element
  source 1..27: mol_type = other DNA
gattgccgcgcgctgtatggcggctgt

SEQ ID NO: 499 - DNA, 27 bp, synthetic construct
  misc_feature 1..27: note = toxin sequence element
  source 1..27: mol_type = other DNA
gattgccgcaaaatgtttggcggctgt

SEQ ID NO: 500 - DNA, 27 bp, synthetic construct
  misc_feature 1..27: note = toxin sequence element
  source 1..27: mol_type = other DNA
gattgcacccgcatgtttggcgcgtgt

SEQ ID NO: 501 - DNA, 27 bp, synthetic construct
  misc_feature 1..27: note = toxin sequence element
  source 1..27: mol_type = other DNA
gattgcgtgcgcttttggggcaaatgt

SEQ ID NO: 502 - DNA, 30 bp, synthetic construct
  misc_feature 1..30: note = toxin sequence element
  source 1..30: mol_type = other DNA
gatgattgcggcggcctgtttagcggctgt

SEQ ID NO: 503 - DNA, 30 bp, synthetic construct
  misc_feature 1..30: note = toxin sequence element
  source 1..30: mol_type = other DNA
gatgattgcggcaaactgtttagcggctgt

SEQ ID NO: 504 - DNA, 30 bp, synthetic construct
  misc_feature 1..30: note = toxin sequence element
  source 1..30: mol_type = other DNA
gatgattgcggcaccctgtttagcggctgt

SEQ ID NO: 505 - DNA, 30 bp, synthetic construct
  misc_feature 1..30: note = toxin sequence element
  source 1..30: mol_type = other DNA
gatgattgcctgggcatgtttagcagctgt

SEQ ID NO: 506 - DNA, 33 bp, synthetic construct
  misc_feature 1..33: note = toxin sequence element
  source 1..33: mol_type = other DNA
gatgatgattgcggctggattatggatgattgt

SEQ ID NO: 507 - DNA, 33 bp, synthetic construct
  misc_feature 1..33: note = toxin sequence element
  source 1..33: mol_type = other DNA
gatggcgaatgcggcggcttttggtggaaatgt

SEQ ID NO: 508 - DNA, 27 bp, synthetic construct
  misc_feature 1..27: note = toxin sequence element
  source 1..27: mol_type = other DNA
gaatgcggcaaatttatgtggaaatgt

SEQ ID NO: 509 - DNA, 27 bp, synthetic construct
  misc_feature 1..27: note = toxin sequence element
  source 1..27: mol_type = other DNA
gaatgcggcaccctgtttagcggctgt

SEQ ID NO: 510 - DNA, 27 bp, synthetic construct
  misc_feature 1..27: note = toxin sequence element
  source 1..27: mol_type = other DNA
gaatgcaaaggctttggcaaaagctgt

SEQ ID NO: 511 - DNA, 27 bp, synthetic construct
  misc_feature 1..27: note = toxin sequence element
  source 1..27: mol_type = other DNA
gaatgcaaaaaactgtttggcggctgt

SEQ ID NO: 512 - DNA, 27 bp, synthetic construct
  misc_feature 1..27: note = toxin sequence element
  source 1..27: mol_type = other DNA
gaatgcaaatggtatctgggcgattgt

SEQ ID NO: 513 - DNA, 27 bp, synthetic construct
  misc_feature 1..27: note = toxin sequence element
  source 1..27: mol_type = other DNA
gaatgcaaatatctgtggggcacctgt

SEQ ID NO: 514 - DNA, 27 bp, synthetic construct
  misc_feature 1..27: note = toxin sequence element
  source 1..27: mol_type = other DNA
gaatgcctggaaatttttaaagcgtgt

SEQ ID NO: 515 - DNA, 27 bp, synthetic construct
  misc_feature 1..27: note = toxin sequence element
  source 1..27: mol_type = other DNA
gaatgcctgggctttggcaaaggctgt

SEQ ID NO: 516 - DNA, 27 bp, synthetic construct
  misc_feature 1..27: note = toxin sequence element
  source 1..27: mol_type = other DNA
gaatgccgcaaaatgtttggcggctgt

SEQ ID NO: 517 - DNA, 26 bp, synthetic construct
  misc_feature 1..26: note = toxin sequence element
  source 1..26: mol_type = other DNA
gaatgccgctggctgtttggcggtgt

SEQ ID NO: 518 - DNA, 27 bp, synthetic construct
  misc_feature 1..27: note = toxin sequence element
  source 1..27: mol_type = other DNA
gaatgccgctggtatctgggcggctgt

SEQ ID NO: 519 - DNA, 27 bp, synthetic construct
  misc_feature 1..27: note = toxin sequence element
  source 1..27: mol_type = other DNA
gaatgccgctatttttggggcgaatgt

SEQ ID NO: 520 - DNA, 27 bp, synthetic construct
  misc_feature 1..27: note = toxin sequence element
  source 1..27: mol_type = other DNA
gaatgccgctatctgtttggcggctgt

SEQ ID NO: 521 - DNA, 27 bp, synthetic construct
  misc_feature 1..27: note = toxin sequence element
  source 1..27: mol_type = other DNA
gaatgccgctattggctgggcggctgt

SEQ ID NO: 522 - DNA, 27 bp, synthetic construct
  misc_feature 1..27: note = toxin sequence element
  source 1..27: mol_type = other DNA
gaatgccgctattggctgggcacctgt

SEQ ID NO: 523 - DNA, 27 bp, synthetic construct
  misc_feature 1..27: note = toxin sequence element
  source 1..27: mol_type = other DNA
gaatgcaccaaatttctgggcggctgt

SEQ ID NO: 524 - DNA, 27 bp, synthetic construct
  misc_feature 1..27: note = toxin sequence element
  source 1..27: mol_type = other DNA
gaatgcaccaaactgctgggcggctgt

SEQ ID NO: 525 - DNA, 30 bp, synthetic construct
  misc_feature 1..30: note = toxin sequence element
  source 1..30: mol_type = other DNA
gaagattgcattccgaaatggaaaggctgt

SEQ ID NO: 526 - DNA, 33 bp, synthetic construct
  misc_feature 1..33: note = toxin sequence element
  source 1..33: mol_type = other DNA
gaaggcgaatgcggcggcttttggtggaaatgt

SEQ ID NO: 527 - DNA, 30 bp, synthetic construct
  misc_feature 1..30: note = toxin sequence element
  source 1..30: mol_type = other DNA
ggcgcgtgccgctggtttctgggcggctgt

SEQ ID NO: 528 - DNA, 27 bp, synthetic construct
  misc_feature 1..27: note = toxin sequence element
  source 1..27: mol_type = other DNA
ggctgcgcgaacgcgtataaaagctgt

SEQ ID NO: 529 - DNA, 27 bp, synthetic construct
  misc_feature 1..27: note = toxin sequence element
  source 1..27: mol_type = other DNA
ggctgcggcggcctgatggcgggctgt

SEQ ID NO: 530 - DNA, 27 bp, synthetic construct
  misc_feature 1..27: note = toxin sequence element
  source 1..27: mol_type = other DNA
ggctgcggcggcctgatggatggctgt

SEQ ID NO: 531 - DNA, 27 bp, synthetic construct
  misc_feature 1..27: note = toxin sequence element
  source 1..27: mol_type = other DNA
ggctgcggcaccatgtggagcccgtgt

SEQ ID NO: 532 - DNA, 27 bp, synthetic construct
  misc_feature 1..27: note = toxin sequence element
  source 1..27: mol_type = other DNA
ggctgcattccgagctttggcgaatgt

SEQ ID NO: 533 - DNA, 27 bp, synthetic construct
  misc_feature 1..27: note = toxin sequence element
  source 1..27: mol_type = other DNA
ggctgcaaaggctttggcgatagctgt

SEQ ID NO: 534 - DNA, 27 bp, synthetic construct
  misc_feature 1..27: note = toxin sequence element
  source 1..27: mol_type = other DNA
ggctgcaaactgaccttttggaaatgt

SEQ ID NO: 535 - DNA, 27 bp, synthetic construct
  misc_feature 1..27: note = toxin sequence element
  source 1..27: mol_type = other DNA
ggctgcctggaattttggtggaaatgt

SEQ ID NO: 536 - DNA, 21 bp, synthetic construct
  misc_feature 1..21: note = toxin sequence element
  source 1..21: mol_type = other DNA
ggctgcctgggcgataaatgt

SEQ ID NO: 537 - DNA, 27 bp, synthetic construct
  misc_feature 1..27: note = toxin sequence element
  source 1..27: mol_type = other DNA
ggctgcaaccgcaaaaacaaaaaatgt

SEQ ID NO: 538 - DNA, 27 bp, synthetic construct
  misc_feature 1..27: note = toxin sequence element
  source 1..27: mol_type = other DNA
ggctgccagaaatttttttggacctgt

SEQ ID NO: 539 - DNA, 30 bp, synthetic construct
  misc_feature 1..30: note = toxin sequence element
  source 1..30: mol_type = other DNA
ggcgattgcctgccgcatctgaaactgtgt

SEQ ID NO: 540 - DNA, 30 bp, synthetic construct
  misc_feature 1..30: note = toxin sequence element
  source 1..30: mol_type = other DNA
ggcgattgcctgccgcatctgaaacgctgt

SEQ ID NO: 541 - DNA, 30 bp, synthetic construct
  misc_feature 1..30: note = toxin sequence element
  source 1..30: mol_type = other DNA
ggcggctgcctgccgcataaccgcttttgt

SEQ ID NO: 542 - DNA, 42 bp, synthetic construct
  misc_feature 1..42: note = toxin sequence element
  source 1..42: mol_type = other DNA
ggcgtggataaagcgggctgccgctatatgtttggcggctgt

SEQ ID NO: 543 - DNA, 42 bp, synthetic construct
  misc_feature 1..42: note = toxin sequence element
  source 1..42: mol_type = other DNA
ggcgtggataaagaaggctgccgcaaactgctgggcggctgt

SEQ ID NO: 544 - DNA, 30 bp, synthetic construct
  misc_feature 1..30: note = toxin sequence element
  source 1..30: mol_type = other DNA
attgcgtgcgcgccgcgctttagcatttgt

SEQ ID NO: 545 - DNA, 30 bp, synthetic construct
  misc_feature 1..30: note = toxin sequence element
  source 1..30: mol_type = other DNA
attgcgtgcgcgccgcgctttagcctgtgt

SEQ ID NO: 546 - DNA, 30 bp, synthetic construct
  misc_feature 1..30: note = toxin sequence element
  source 1..30: mol_type = other DNA
attgcgtgcgcgccgcgcggcctgctgtgt

SEQ ID NO: 547 - DNA, 27 bp, synthetic construct
  misc_feature 1..27: note = toxin sequence element
  source 1..27: mol_type = other DNA
aaatgcctgccgccgggcaaaccgtgt

SEQ ID NO: 548 - DNA, 24 bp, synthetic construct
  misc_feature 1..24: note = toxin sequence element
  source 1..24: mol_type = other DNA
ctgtgcagccgcgaaggcgaattt

SEQ ID NO: 549 - DNA, 27 bp, synthetic construct
  misc_feature 1..27: note = toxin sequence element
  source 1..27: mol_type = other DNA
cagtgcggcgaatttatgtggaaatgt

SEQ ID NO: 550 - DNA, 27 bp, synthetic construct
  misc_feature 1..27: note = toxin sequence element
  source 1..27: mol_type = other DNA
cgctgcattgaagaaggcaaatggtgt

SEQ ID NO: 551 - DNA, 33 bp, synthetic construct
  misc_feature 1..33: note = toxin sequence element
  source 1..33: mol_type = other DNA
agcgcggtgtgcattccgagcggccagccgtgt

SEQ ID NO: 552 - DNA, 27 bp, synthetic construct
  misc_feature 1..27: note = toxin sequence element
  source 1..27: mol_type = other DNA
agctgcaaactgaccttttggcgctgt

SEQ ID NO: 553 - DNA, 30 bp, synthetic construct
  misc_feature 1..30: note = toxin sequence element
  source 1..30: mol_type = other DNA
agcgaatgccgctggtttatgggcggctgt

SEQ ID NO: 554 - DNA, 36 bp, synthetic construct
  misc_feature 1..36: note = toxin sequence element
  source 1..36: mol_type = other DNA
agcgaaaaagattgcattaaacatctgcagcgctgt

SEQ ID NO: 555 - DNA, 27 bp, synthetic construct
  misc_feature 1..27: note = toxin sequence element
  source 1..27: mol_type = other DNA
agcttttgcattccgtttaaaccgtgt

SEQ ID NO: 556 - DNA, 33 bp, synthetic construct
  misc_feature 1..33: note = toxin sequence element
  source 1..33: mol_type = other DNA
agcccgacctgcattccgagcggccagccgtgt

SEQ ID NO: 557 - DNA, 33 bp, synthetic construct
  misc_feature 1..33: note = toxin sequence element
  source 1..33: mol_type = other DNA
agcccgacctgcattccgaccggccagccgtgt

SEQ ID NO: 558 - DNA, 33 bp, synthetic construct
  misc_feature 1..33: note = toxin sequence element
  source 1..33: mol_type = other DNA
agcccgacctgcattcgcagcggccagccgtgt

SEQ ID NO: 559 - DNA, 33 bp, synthetic construct
  misc_feature 1..33: note = toxin sequence element
  source 1..33: mol_type = other DNA
agcccggtgtgcaccccgagcggccagccgtgt

SEQ ID NO: 560 - DNA, 33 bp, synthetic construct
  misc_feature 1..33: note = toxin sequence element
  source 1..33: mol_type = other DNA
agcagcacctgcattccgagcggccagccgtgt

SEQ ID NO: 561 - DNA, 33 bp, synthetic construct
  misc_feature 1..33: note = toxin sequence element
  source 1..33: mol_type = other DNA
cggctggccgctcggaatgcaggtgctgcttgt

SEQ ID NO: 562 - DNA, 30 bp, synthetic construct
  misc_feature 1..30: note = toxin sequence element
  source 1..30: mol_type = other DNA
agcacctgcaccccgaccgatcagccgtgt

SEQ ID NO: 563 - DNA, 30 bp, synthetic construct
  misc_feature 1..30: note = toxin sequence element
  source 1..30: mol_type = other DNA
agcgtgtgcattccgagcggccagccgtgt

SEQ ID NO: 564 - DNA, 27 bp, synthetic construct
  misc_feature 1..27: note = toxin sequence element
  source 1..27: mol_type = other DNA
acctgccgctatctgtttggcggctgt

SEQ ID NO: 565 - DNA, 27 bp, synthetic construct
  misc_feature 1..27: note = toxin sequence element
  source 1..27: mol_type = other DNA
acctgctatgatattggcgaactgtgt

SEQ ID NO: 566 - DNA, 27 bp, synthetic construct
  misc_feature 1..27: note = toxin sequence element
  source 1..27: mol_type = other DNA
gtgtgccgcggctatggcctgccgtgt

SEQ ID NO: 567 - DNA, 27 bp, synthetic construct
  misc_feature 1..27: note = toxin sequence element
  source 1..27: mol_type = other DNA
tattgccagaaatggctgtggacctgt

SEQ ID NO: 568 - DNA, 27 bp, synthetic construct
  misc_feature 1..27: note = toxin sequence element
  source 1..27: mol_type = other DNA
tattgccagaaatggatgtggacctgt

SEQ ID NO: 569 - DNA, 24 bp, synthetic construct
  misc_feature 1..24: note = toxin sequence element
  source 1..24: mol_type = other DNA
tgcaaacaggcggatgaaccgtgt

SEQ ID NO: 570 - DNA, 27 bp, synthetic construct
  misc_feature 1..27: note = toxin sequence element
  source 1..27: mol_type = other DNA
gcgtgccgcaaaaaatgggaatattgt

SEQ ID NO: 571 - DNA, 33 bp, synthetic construct
  misc_feature 1..33: note = toxin sequence element
  source 1..33: mol_type = other DNA
gatgatgattgcgaaccgccgggcaacttttgt

SEQ ID NO: 572 - DNA, 33 bp, synthetic construct
  misc_feature 1..33: note = toxin sequence element
  source 1..33: mol_type = other DNA
gtgaaaccgtgccgcaaagaaggccagctgtgt

SEQ ID NO: 573 - DNA, 27 bp, synthetic construct
  misc_feature 1..27: note = toxin sequence element
  source 1..27: mol_type = other DNA
tggtgcaaacagagcggcgaaatgtgt

SEQ ID NO: 574 - DNA, 24 bp, synthetic construct
  misc_feature 1..24: note = toxin sequence element
  source 1..24: mol_type = other DNA
tgcctgagcggcggcgaagtgtgt

SEQ ID NO: 575 - DNA, 33 bp, synthetic construct
  misc_feature 1..33: note = toxin sequence element
  source 1..33: mol_type = other DNA
ggcaaaccgtgccatgaagaaggccagctgtgt

SEQ ID NO: 576 - DNA, 24 bp, synthetic construct
  misc_feature 1..24: note = toxin sequence element
  source 1..24: mol_type = other DNA
tgcattccgtttctgcatccgtgt

SEQ ID NO: 577 - DNA, 27 bp, synthetic construct
  misc_feature 1..27: note = toxin sequence element
  source 1..27: mol_type = other DNA
gcgtgcagcaaaaaatgggaatattgt

SEQ ID NO: 578 - DNA, 36 bp, synthetic construct
  misc_feature 1..36: note = toxin sequence element
  source 1..36: mol_type = other DNA
gcggatagcgatgattgctgcgaaacctttcattgc

SEQ ID NO: 579 - DNA, 39 bp, synthetic construct
  misc_feature 1..39: note = toxin sequence element
  source 1..39: mol_type = other DNA
gcgtggtttagcggcgaaagctgctgcaccggcatttgc

SEQ ID NO: 580 - DNA, 36 bp, synthetic construct
  misc_feature 1..36: note = toxin sequence element
  source 1..36: mol_type = other DNA
gatgaagaacgcaaatgctgcgaaggcctggtgtgc

SEQ ID NO: 581 - DNA, 39 bp, synthetic construct
  misc_feature 1..39: note = toxin sequence element
  source 1..39: mol_type = other DNA
gatgaaaacgatccgcgctgctgcagcggcctggtgtgc

SEQ ID NO: 582 - DNA, 39 bp, synthetic construct
  misc_feature 1..39: note = toxin sequence element
  source 1..39: mol_type = other DNA
gatggcaaaagcaccttttgctgcagcggctttaactgc

SEQ ID NO: 583 - DNA, 39 bp, synthetic construct
  misc_feature 1..39: note = toxin sequence element
  source 1..39: mol_type = other DNA
gatggcaaaagcaccttttgctgcagcggctataactgc

SEQ ID NO: 584 - DNA, 39 bp, synthetic construct
  misc_feature 1..39: note = toxin sequence element
  source 1..39: mol_type = other DNA
gatccggataacgataaatgctgcgaaggctataaatgc

SEQ ID NO: 585 - DNA, 39 bp, synthetic construct
  misc_feature 1..39: note = toxin sequence element
  source 1..39: mol_type = other DNA
gatccgaaaaacgataaatgctgcaaaaactatacctgc

SEQ ID NO: 586 - DNA, 39 bp, synthetic construct
  misc_feature 1..39: note = toxin sequence element
  source 1..39: mol_type = other DNA
gatccgaaaaacgataaatgctgcccgaaccgcgtgtgc

SEQ ID NO: 587 - DNA, 39 bp, synthetic construct
  misc_feature 1..39: note = toxin sequence element
  source 1..39: mol_type = other DNA
gatccgaacaacgataaatgctgcccgaaccgcgaatgc

SEQ ID NO: 588 - DNA, 39 bp, synthetic construct
  misc_feature 1..39: note = toxin sequence element
  source 1..39: mol_type = other DNA
gatccgaacaacgataaatgctgcccgaaccgcgtgtgc

SEQ ID NO: 589 - DNA, 48 bp, synthetic construct
  misc_feature 1..48: note = toxin sequence element
  source 1..48: mol_type = other DNA
gatccgaacccggtgaaagatctgccgtgctgcagcggcctggcgtgc

SEQ ID NO: 590 - DNA, 36 bp, synthetic construct
  misc_feature 1..36: note = toxin sequence element
  source 1..36: mol_type = other DNA
gatagcgcgcgcaaatgctgcgaaggcctggtgtgc

SEQ ID NO: 591 - DNA, 36 bp, synthetic construct
  misc_feature 1..36: note = toxin sequence element
  source 1..36: mol_type = other DNA
gatagcgaacgcaaatgctgcgaagatatggtgtgc

SEQ ID NO: 592 - DNA, 36 bp, synthetic construct
  misc_feature 1..36: note = toxin sequence element
  source 1..36: mol_type = other DNA
gatagcgaacgcaaatgctgcgaaggcatggtgtgc

SEQ ID NO: 593 - DNA, 36 bp, synthetic construct
  misc_feature 1..36: note = toxin sequence element
  source 1..36: mol_type = other DNA
gatagcgaacgcaaatgctgcgaaggctatgtgtgc

SEQ ID NO: 594 - DNA, 36 bp, synthetic construct
  misc_feature 1..36: note = toxin sequence element
  source 1..36: mol_type = other DNA
gatagcaaacgcgcgtgctgcgaaggcctgcgctgc

SEQ ID NO: 595 - DNA, 36 bp, synthetic construct
  misc_feature 1..36: note = toxin sequence element
  source 1..36: mol_type = other DNA
gatagcaaacgcaaatgctgcgaagatatggtgtgc

SEQ ID NO: 596 - DNA, 36 bp, synthetic construct
  misc_feature 1..36: note = toxin sequence element
  source 1..36: mol_type = other DNA
gatagcaacgcggattgctgcgaaggctatgtgtgc

SEQ ID NO: 597 - DNA, 36 bp, synthetic construct
  misc_feature 1..36: note = toxin sequence element
  source 1..36: mol_type = other DNA
gatagcaccctggattgctgcaaacatctgagctgc

SEQ ID NO: 598 - DNA, 36 bp, synthetic construct
  misc_feature 1..36: note = toxin sequence element
  source 1..36: mol_type = other DNA
gataccaacgcggattgctgcgaaggctatgtgtgc

SEQ ID NO: 599 - DNA, 36 bp, synthetic construct
  misc_feature 1..36: note = toxin sequence element
  source 1..36: mol_type = other DNA
gataccagcaaagattgctgcgaaggctatgtgtgc

SEQ ID NO: 600 - DNA, 36 bp, synthetic construct
  misc_feature 1..36: note = toxin sequence element
  source 1..36: mol_type = other DNA
gattataacaacggctgctgcagcggctatgtgtgc

SEQ ID NO: 601 - DNA, 36 bp, synthetic construct
  misc_feature 1..36: note = toxin sequence element
  source 1..36: mol_type = other DNA
gaaaaagatgaacattgctgcgaacatctgggctgc

SEQ ID NO: 602 - DNA, 36 bp, synthetic construct
  misc_feature 1..36: note = toxin sequence element
  source 1..36: mol_type = other DNA
gaaaaagatagcgattgctgcgaacatctgggctgc

SEQ ID NO: 603 - DNA, 36 bp, synthetic construct
  misc_feature 1..36: note = toxin sequence element
  source 1..36: mol_type = other DNA
gaaagcaacgcggattgctgcgaaaactgggcgtgc

SEQ ID NO: 604 - DNA, 36 bp, synthetic construct
  misc_feature 1..36: note = toxin sequence element
  source 1..36: mol_type = other DNA
tttcgcgataaagaatgctgcaaaggcctgacctgc

SEQ ID NO: 605 - DNA, 39 bp, synthetic construct
  misc_feature 1..39: note = toxin sequence element
  source 1..39: mol_type = other DNA
ggcgcgggcaaaccgacctgctgcagcggctatgattgc

SEQ ID NO: 606 - DNA, 39 bp, synthetic construct
  misc_feature 1..39: note = toxin sequence element
  source 1..39: mol_type = other DNA
ggcgaaggcaaaccgccgtgctgcgcgaactttgcgtgc

SEQ ID NO: 607 - DNA, 39 bp, synthetic construct
  misc_feature 1..39: note = toxin sequence element
  source 1..39: mol_type = other DNA
ggccgcggcaaaccgccgtgctgcaaaggctatgcgtgc

SEQ ID NO: 608 - DNA, 39 bp, synthetic construct
  misc_feature 1..39: note = toxin sequence element
  source 1..39: mol_type = other DNA
ggcagcggcaaaccggcgtgctgcccgaaatatgtgtgc

SEQ ID NO: 609 - DNA, 39 bp, synthetic construct
  misc_feature 1..39: note = toxin sequence element
  source 1..39: mol_type = other DNA
ggcaccgatcagagcgaatgctgcgaaggctggaaatgc

SEQ ID NO: 610 - DNA, 39 bp, synthetic construct
  misc_feature 1..39: note = toxin sequence element
  source 1..39: mol_type = other DNA
catccgggccagccgccgtgctgcagcggcctggcgtgc

SEQ ID NO: 611 - DNA, 33 bp, synthetic construct
  misc_feature 1..33: note = toxin sequence element
  source 1..33: mol_type = other DNA
aaagcggataacgattgctgcggcaaaaaatgc

SEQ ID NO: 612 - DNA, 36 bp, synthetic construct
  misc_feature 1..36: note = toxin sequence element
  source 1..36: mol_type = other DNA
aaagcgcatgaagattgctgcgaacatctgcgctgc

SEQ ID NO: 613 - DNA, 36 bp, synthetic construct
  misc_feature 1..36: note = toxin sequence element
  source 1..36: mol_type = other DNA
aaagaagatagcgaatgctgcgaacatctgcagtgc

SEQ ID NO: 614 - DNA, 33 bp, synthetic construct
  misc_feature 1..33: note = toxin sequence element
  source 1..33: mol_type = other DNA
aaagaaaaactgtgctgcagcggctatgtgtgc

SEQ ID NO: 615 - DNA, 33 bp, synthetic construct
  misc_feature 1..33: note = toxin sequence element
  source 1..33: mol_type = other DNA
aaagaaaacaaagattgctgcagcaaaaaatgc

SEQ ID NO: 616 - DNA, 36 bp, synthetic construct
  misc_feature 1..36: note = toxin sequence element
  source 1..36: mol_type = other DNA
aaaaaagataaagaatgctgcggctggaacatttgc

SEQ ID NO: 617 - DNA, 36 bp, synthetic construct
  misc_feature 1..36: note = toxin sequence element
  source 1..36: mol_type = other DNA
aaaaacaaaaaagaatgctgcggctggaacgcgtgc

SEQ ID NO: 618 - DNA, 36 bp, synthetic construct
  misc_feature 1..36: note = toxin sequence element
  source 1..36: mol_type = other DNA
aaaaacagcaacgattgctgcaaagatctggtgtgc

SEQ ID NO: 619 - DNA, 36 bp, synthetic construct
  misc_feature 1..36: note = toxin sequence element
  source 1..36: mol_type = other DNA
aaaagcgatgaaaactgctgcaaaaaatttaaatgc

SEQ ID NO: 620 - DNA, 36 bp, synthetic construct
  misc_feature 1..36: note = toxin sequence element
  source 1..36: mol_type = other DNA
aaaagcaccagcgattgctgcgaacatctgagctgc

SEQ ID NO: 621 - DNA, 36 bp, synthetic construct
  misc_feature 1..36: note = toxin sequence element
  source 1..36: mol_type = other DNA
aaaaccaccgcggattgctgcaaacatctggcgtgc

SEQ ID NO: 622 - DNA, 36 bp, synthetic construct
  misc_feature 1..36: note = toxin sequence element
  source 1..36: mol_type = other DNA
aaaaccaccgcggattgctgcaaacatctgggctgc

SEQ ID NO: 623 - DNA, 36 bp, synthetic construct
  misc_feature 1..36: note = toxin sequence element
  source 1..36: mol_type = other DNA
aaaaccaccagcgattgctgcaaacatctgggctgc

SEQ ID NO: 624 - DNA, 42 bp, synthetic construct
  misc_feature 1..42: note = toxin sequence element
  source 1..42: mol_type = other DNA
aacgcgctgagcggcccgcgctgctgcagcggcctgaaatgc

SEQ ID NO: 625 - DNA, 36 bp, synthetic construct
  misc_feature 1..36: note = toxin sequence element
  source 1..36: mol_type = other DNA
aacgatgaaatggtgtgctgcgaacatctggtgtgc

SEQ ID NO: 626 - DNA, 51 bp, synthetic construct
  misc_feature 1..51: note = toxin sequence element
  source 1..51: mol_type = other DNA
aacggcccgcatacctgctgctggggctataacggctataaaaaagcgtgc

SEQ ID NO: 627 - DNA, 39 bp, synthetic construct
  misc_feature 1..39: note = toxin sequence element
  source 1..39: mol_type = other DNA
aacccggataacgataaatgctgcgaaggccgcaaatgc

SEQ ID NO: 628 - DNA, 42 bp, synthetic construct
  misc_feature 1..42: note = toxin sequence element
  source 1..42: mol_type = other DNA
aacccgaacgatgataaatgctgccgcccgaaactgaaatgc

SEQ ID NO: 629 - DNA, 42 bp, synthetic construct
  misc_feature 1..42: note = toxin sequence element
  source 1..42: mol_type = other DNA
aacccgagcaacgataaatgctgccgcccgaacctggtgtgc

SEQ ID NO: 630 - DNA, 45 bp, synthetic construct
  misc_feature 1..45: note = toxin sequence element
  source 1..45: mol_type = other DNA
aacccgagcaacgatcagtgctgcaaaagcgcgaacctggtgtgc

SEQ ID NO: 631 - DNA, 45 bp, synthetic construct
  misc_feature 1..45: note = toxin sequence element
  source 1..45: mol_type = other DNA
aacccgagcaacgatcagtgctgcaaaagcagcaaactggtgtgc

SEQ ID NO: 632 - DNA, 45 bp, synthetic construct
  misc_feature 1..45: note = toxin sequence element
  source 1..45: mol_type = other DNA
aacccgagcaacgatcagtgctgcaaaagcagcaacctggtgtgc

SEQ ID NO: 633 - DNA, 39 bp, synthetic construct
  misc_feature 1..39: note = toxin sequence element
  source 1..39: mol_type = other DNA
aacagcgatgcggattgctgccgctatggcgaacgctgc

SEQ ID NO: 634 - DNA, 36 bp, synthetic construct
  misc_feature 1..36: note = toxin sequence element
  source 1..36: mol_type = other DNA
aacagcgataaagaatgctgcaaaggcctgcgctgc

SEQ ID NO: 635 - DNA, 39 bp, synthetic construct
  misc_feature 1..39: note = toxin sequence element
  source 1..39: mol_type = other DNA
aactatatggatgataaatgctgcccgggctataaatgc

SEQ ID NO: 636 - DNA, 36 bp, synthetic construct
  misc_feature 1..36: note = toxin sequence element
  source 1..36: mol_type = other DNA
ccgaaaaaagcgccgtgctgcggccgcctggaatgc

SEQ ID NO: 637 - DNA, 33 bp, synthetic construct
  misc_feature 1..33: note = toxin sequence element
  source 1..33: mol_type = other DNA
ccgtatcatgaaagctgctgcagcggcagctgc

SEQ ID NO: 638 - DNA, 33 bp, synthetic construct
  misc_feature 1..33: note = toxin sequence element
  source 1..33: mol_type = other DNA
ccgtataacgaacattgctgcagcggcagctgc

SEQ ID NO: 639 - DNA, 33 bp, synthetic construct
  misc_feature 1..33: note = toxin sequence element
  source 1..33: mol_type = other DNA
ccgtataacgaaaactgctgcagcaaaagctgc

SEQ ID NO: 640 - DNA, 33 bp, synthetic construct
  misc_feature 1..33: note = toxin sequence element
  source 1..33: mol_type = other DNA
ccgtataacgaaaactgctgcagccagagctgc

SEQ ID NO: 641 - DNA, 33 bp, synthetic construct
  misc_feature 1..33: note = toxin sequence element
  source 1..33: mol_type = other DNA
ccgtataacgaaagctgctgcagcggcagctgc

SEQ ID NO: 642 - DNA, 33 bp, synthetic construct
  misc_feature 1..33: note = toxin sequence element
  source 1..33: mol_type = other DNA
ccgtatagcaaatattgctgcagcggcagctgc

SEQ ID NO: 643 - DNA, 57 bp, synthetic construct
  misc_feature 1..57: note = toxin sequence element
  source 1..57: mol_type = other DNA
cagccgaacacccagccgtgctgcaacaacgcggaagaagaacagaccattaactgc

SEQ ID NO: 644 - DNA, 33 bp, synthetic construct
  misc_feature 1..33: note = toxin sequence element
  source 1..33: mol_type = other DNA
cgcgaaaacaaagattgctgcagcaaaaaatgc

SEQ ID NO: 645 - DNA, 36 bp, synthetic construct
  misc_feature 1..36: note = toxin sequence element
  source 1..36: mol_type = other DNA
cgccgcgatagcgattgctgcccgcatctgggctgc

SEQ ID NO: 646 - DNA, 36 bp, synthetic construct
  misc_feature 1..36: note = toxin sequence element
  source 1..36: mol_type = other DNA
agcgcgggccagacctgctgcaaacatctggtgtgc

SEQ ID NO: 647 - DNA, 36 bp, synthetic construct
  misc_feature 1..36: note = toxin sequence element
  source 1..36: mol_type = other DNA
agcgaagatagcgaatgctgcccgcatctgggctgc

SEQ ID NO: 648 - DNA, 36 bp, synthetic construct
  misc_feature 1..36: note = toxin sequence element
  source 1..36: mol_type = other DNA
agcaaagatgcggattgctgcgcgcatctggaatgc

SEQ ID NO: 649 - DNA, 36 bp, synthetic construct
  misc_feature 1..36: note = toxin sequence element
  source 1..36: mol_type = other DNA
agcaaacatgaagattgctgcgcgcatctggcgtgc

SEQ ID NO: 650 - DNA, 36 bp, synthetic construct
  misc_feature 1..36: note = toxin sequence element
  source 1..36: mol_type = other DNA
agcaaaaccggcgattgctgcagccatctgagctgc

SEQ ID NO: 651 - DNA, 36 bp, synthetic construct
  misc_feature 1..36: note = toxin sequence element
  source 1..36: mol_type = other DNA
agccaggatggcgattgctgcaaacatctgcagtgc

SEQ ID NO: 652 - DNA, 36 bp, synthetic construct
  misc_feature 1..36: note = toxin sequence element
  source 1..36: mol_type = other DNA
agccagaccagcgattgctgcccgcatctggcgtgc

SEQ ID NO: 653 - DNA, 36 bp, synthetic construct
  misc_feature 1..36: note = toxin sequence element
  source 1..36: mol_type = other DNA
agcagcgataaaccgtgctgcagcggctattattgc

SEQ ID NO: 654 - DNA, 36 bp, synthetic construct
  misc_feature 1..36: note = toxin sequence element
  source 1..36: mol_type = other DNA
agcagcaccagcgattgctgcaaacatctgagctgc

SEQ ID NO: 655 - DNA, 36 bp, synthetic construct
  misc_feature 1..36: note = toxin sequence element
  source 1..36: mol_type = other DNA
agcaccgaaaaaccgtgctgcgataactttagctgc

SEQ ID NO: 656 - DNA, 36 bp, synthetic construct
  misc_feature 1..36: note = toxin sequence element
  source 1..36: mol_type = other DNA
agcacccatgcggattgctgcgaaggctttatttgc

SEQ ID NO: 657 - DNA, 36 bp, synthetic construct
  misc_feature 1..36: note = toxin sequence element
  source 1..36: mol_type = other DNA
agcgtggatagcgattgctgcgcgcatctgggctgc

SEQ ID NO: 658 - DNA, 36 bp, synthetic construct
  misc_feature 1..36: note = toxin sequence element
  source 1..36: mol_type = other DNA
agcgtgcatagcgattgctgcgcgcatctgggctgc

SEQ ID NO: 659 - DNA, 36 bp, synthetic construct
  misc_feature 1..36: note = toxin sequence element
  source 1..36: mol_type = other DNA
agcgtgaacgatgattgctgcccgcgcctgggctgc

SEQ ID NO: 660 - DNA, 36 bp, synthetic construct
  misc_feature 1..36: note = toxin sequence element
  source 1..36: mol_type = other DNA
accgatcgcctgccgtgctgctttggcctggaatgc

SEQ ID NO: 661 - DNA, 36 bp, synthetic construct
  misc_feature 1..36: note = toxin sequence element
  source 1..36: mol_type = other DNA
accattgatgatgattgctgcccgcatctgggctgc

SEQ ID NO: 662 - DNA, 36 bp, synthetic construct
  misc_feature 1..36: note = toxin sequence element
  source 1..36: mol_type = other DNA
accaaagatgaagattgctgcaaacatctggcgtgc

SEQ ID NO: 663 - DNA, 36 bp, synthetic construct
  misc_feature 1..36: note = toxin sequence element
  source 1..36: mol_type = other DNA
accaaagatagcgaatgctgcccgcatctgggctgc

SEQ ID NO: 664 - DNA, 39 bp, synthetic construct
  misc_feature 1..39: note = toxin sequence element
  source 1..39: mol_type = other DNA
accccggaaaaaaacgattgctgccagcgcctgtattgc

SEQ ID NO: 665 - DNA, 39 bp, synthetic construct
  misc_feature 1..39: note = toxin sequence element
  source 1..39: mol_type = other DNA
accccgggcaaaaacgaatgctgcccgaaccgcgtgtgc

SEQ ID NO: 666 - DNA, 39 bp, synthetic construct
  misc_feature 1..39: note = toxin sequence element
  source 1..39: mol_type = other DNA
accccgggcaaaaacgaatgctgcccgaactatgcgtgc

SEQ ID NO: 667 - DNA, 36 bp, synthetic construct
  misc_feature 1..36: note = toxin sequence element
  source 1..36: mol_type = other DNA
accagcgatagcgattgctgcccgaactgggtgtgc

SEQ ID NO: 668 - DNA, 36 bp, synthetic construct
  misc_feature 1..36: note = toxin sequence element
  source 1..36: mol_type = other DNA
accaccagcagcgaatgctgcgcgcatctgggctgc

SEQ ID NO: 669 - DNA, 39 bp, synthetic construct
  misc_feature 1..39: note = toxin sequence element
  source 1..39: mol_type = other DNA
gtgaaccgccatggcgattgctgcgaaggcctggaatgc

SEQ ID NO: 670 - DNA, 39 bp, synthetic construct
  misc_feature 1..39: note = toxin sequence element
  source 1..39: mol_type = other DNA
gtgccgggcaaaaacgaatgctgcagcggctatgcgtgc

SEQ ID NO: 671 - DNA, 39 bp, synthetic construct
  misc_feature 1..39: note = toxin sequence element
  source 1..39: mol_type = other DNA
tatggcgcgacccagaaaattccgtgctgcggcgtgtgc

SEQ ID NO: 672 - DNA, 36 bp, synthetic construct
  misc_feature 1..36: note = toxin sequence element
  source 1..36: mol_type = other DNA
tataaactgcgcaaatgctgcgcgggcttttattgc

SEQ ID NO: 673 - DNA, 36 bp, synthetic construct
  misc_feature 1..36: note = toxin sequence element
  source 1..36: mol_type = other DNA
gatgtgtttagcctggattgctgcaccggcatttgc

SEQ ID NO: 674 - DNA, 48 bp, synthetic construct
  misc_feature 1..48: note = toxin sequence element
  source 1..48: mol_type = other DNA
attgtgccgattattggctttatttattgctgcccgggcctgatttgc

SEQ ID NO: 675 - DNA, 42 bp, synthetic construct
  misc_feature 1..42: note = toxin sequence element
  source 1..42: mol_type = other DNA
ggcatgattaaaattggcccgccgtgctgcagcggctggtgc

SEQ ID NO: 676 - DNA, 39 bp, synthetic construct
  misc_feature 1..39: note = toxin sequence element
  source 1..39: mol_type = other DNA
gatccgatttttcagaactgctgccgcggctggaactgc

SEQ ID NO: 677 - DNA, 36 bp, synthetic construct
  misc_feature 1..36: note = toxin sequence element
  source 1..36: mol_type = other DNA
aacgtgctggatcagaactgctgcgatggctattgc

SEQ ID NO: 678 - DNA, 33 bp, synthetic construct
  misc_feature 1..33: note = toxin sequence element
  source 1..33: mol_type = other DNA
gattttctgtttccgaaatgctgcaactattgc

SEQ ID NO: 679 - DNA, 39 bp, synthetic construct
  misc_feature 1..39: note = toxin sequence element
  source 1..39: mol_type = other DNA
gatccgtttctgcagaactgctgcctgggctggaactgc

SEQ ID NO: 680 - DNA, 36 bp, synthetic construct
  misc_feature 1..36: note = toxin sequence element
  source 1..36: mol_type = other DNA
acctttttttttccggattgctgcaacagcatttgc

SEQ ID NO: 681 - DNA, 48 bp, synthetic construct
  misc_feature 1..48: note = toxin sequence element
  source 1..48: mol_type = other DNA
attgtgccgattctgggctttgtgtattgctgcccgggcctgatttgc

SEQ ID NO: 682 - DNA, 24 bp, synthetic construct
  misc_feature 1..24: note = toxin sequence element
  source 1..24: mol_type = other DNA
gcgctgggcatttgcatgccgcgc

SEQ ID NO: 683 - DNA, 42 bp, synthetic construct
  misc_feature 1..42: note = toxin sequence element
  source 1..42: mol_type = other DNA
gcgaccaccggccgctttcgctatctgtgcaaatatcagatt

SEQ ID NO: 684 - DNA, 36 bp, synthetic construct
  misc_feature 1..36: note = toxin sequence element
  source 1..36: mol_type = other DNA
gatcgccgcgatcagtggtgcaaatggaacccgtgg

SEQ ID NO: 685 - DNA, 24 bp, synthetic construct
  misc_feature 1..24: note = toxin sequence element
  source 1..24: mol_type = other DNA
gaactgtggtgcaaatataacctg

SEQ ID NO: 686 - DNA, 36 bp, synthetic construct
  misc_feature 1..36: note = toxin sequence element
  source 1..36: mol_type = other DNA
gaacgcagcagcccgtggtgcaaaattgatatttgg

SEQ ID NO: 687 - DNA, 21 bp, synthetic construct
  misc_feature 1..21: note = toxin sequence element
  source 1..21: mol_type = other DNA
catctgtggtgcaaatataaa

SEQ ID NO: 688 - DNA, 45 bp, synthetic construct
  misc_feature 1..45: note = toxin sequence element
  source 1..45: mol_type = other DNA
catagcctgtttagctattgcgcgtgggatctgacctttagcgat

SEQ ID NO: 689 - DNA, 45 bp, synthetic construct
  misc_feature 1..45: note = toxin sequence element
  source 1..45: mol_type = other DNA
catagcaactatgaatggtgcatttgggatggcacctttagcaaa

SEQ ID NO: 690 - DNA, 36 bp, synthetic construct
  misc_feature 1..36: note = toxin sequence element
  source 1..36: mol_type = other DNA
catagcaactatgaatggtgcgtgtgggatggcacc

SEQ ID NO: 691 - DNA, 39 bp, synthetic construct
  misc_feature 1..39: note = toxin sequence element
  source 1..39: mol_type = other DNA
catagccgctgggattggtgcatttgggatggcaccttt

SEQ ID NO: 692 - DNA, 39 bp, synthetic construct
  misc_feature 1..39: note = toxin sequence element
  source 1..39: mol_type = other DNA
catagctattgggaatggtgcctgtgggatggcagcttt

SEQ ID NO: 693 - DNA, 21 bp, synthetic construct
  misc_feature 1..21: note = toxin sequence element
  source 1..21: mol_type = other DNA
atttgcagcggcaactggaaa

SEQ ID NO: 694 - DNA, 42 bp, synthetic construct
  misc_feature 1..42: note = toxin sequence element
  source 1..42: mol_type = other DNA
attagcaccaaagtgaactattatcgcccggatcgcggcccg

SEQ ID NO: 695 - DNA, 30 bp, synthetic construct
  misc_feature 1..30: note = toxin sequence element
  source 1..30: mol_type = other DNA
aaagcgtttgtgctgcattgctatcgcaac

SEQ ID NO: 696 - DNA, 39 bp, synthetic construct
  misc_feature 1..39: note = toxin sequence element
  source 1..39: mol_type = other DNA
aaagatgtgctgtattattgcgcgtgggatggcaccttt

SEQ ID NO: 697 - DNA, 51 bp, synthetic construct
  misc_feature 1..51: note = toxin sequence element
  source 1..51: mol_type = other DNA
aaagaaaaatggccgattacctataaaatttgcgtgtgggatcgcaccttt

SEQ ID NO: 698 - DNA, 33 bp, synthetic construct
  misc_feature 1..33: note = toxin sequence element
  source 1..33: mol_type = other DNA
aaagaactgagcatttgggatagccgctgcctg

SEQ ID NO: 699 - DNA, 42 bp, synthetic construct
  misc_feature 1..42: note = toxin sequence element
  source 1..42: mol_type = other DNA
aaatttcgcgataaatattgcgcgtgggattttacctttagc

SEQ ID NO: 700 - DNA, 39 bp, synthetic construct
  misc_feature 1..39: note = toxin sequence element
  source 1..39: mol_type = other DNA
aaaggcccgagcccgaaacagaaaaaatgcacccgcccg

SEQ ID NO: 701 - DNA, 42 bp, synthetic construct
  misc_feature 1..42: note = toxin sequence element
  source 1..42: mol_type = other DNA
aaaggccgctttgtgaacacctggccgaccttttgcctggtg

SEQ ID NO: 702 - DNA, 33 bp, synthetic construct
  misc_feature 1..33: note = toxin sequence element
  source 1..33: mol_type = other DNA
aaaattggcctgtatctgtgcatttggagcccg

SEQ ID NO: 703 - DNA, 27 bp, synthetic construct
  misc_feature 1..27: note = toxin sequence element
  source 1..27: mol_type = other DNA
aaactgtggtgccgcaaaattattggc

SEQ ID NO: 704 - DNA, 30 bp, synthetic construct
  misc_feature 1..30: note = toxin sequence element
  source 1..30: mol_type = other DNA
aaactgtggtgccgctatgaacgcacctgg

SEQ ID NO: 705 - DNA, 39 bp, synthetic construct
  misc_feature 1..39: note = toxin sequence element
  source 1..39: mol_type = other DNA
aaaatgggcctggattattgcgcgtgggatggcaccttt

SEQ ID NO: 706 - DNA, 39 bp, synthetic construct
  misc_feature 1..39: note = toxin sequence element
  source 1..39: mol_type = other DNA
aaaatgggcctgtattattgcgcgtgggatggcaccttt

SEQ ID NO: 707 - DNA, 36 bp, synthetic construct
  misc_feature 1..36: note = toxin sequence element
  source 1..36: mol_type = other DNA
aaaccgaccctgaaatattgcgcgtgggatggcacc

SEQ ID NO: 708 - DNA, 39 bp, synthetic construct
  misc_feature 1..39: note = toxin sequence element
  source 1..39: mol_type = other DNA
aaaccgaccctgaaatattgcgcgtgggatggcaccttt

SEQ ID NO: 709 - DNA, 39 bp, synthetic construct
  misc_feature 1..39: note = toxin sequence element
  source 1..39: mol_type = other DNA
aaaccgaccagcaaatattgcgcgtgggatggcaccatt

SEQ ID NO: 710 - DNA, 42 bp, synthetic construct
  misc_feature 1..42: note = toxin sequence element
  source 1..42: mol_type = other DNA
aaacagaaatggccgttttattgcgcgtgggattggagcttt

SEQ ID NO: 711 - DNA, 36 bp, synthetic construct
  misc_feature 1..36: note = toxin sequence element
  source 1..36: mol_type = other DNA
aaacgccgcggcaccaacgcggaaaaacgctgccgc

SEQ ID NO: 712 - DNA, 36 bp, synthetic construct
  misc_feature 1..36: note = toxin sequence element
  source 1..36: mol_type = other DNA
aaacgccgcggcaccaacattgaaaaacgctgccgc

SEQ ID NO: 713 - DNA, 45 bp, synthetic construct
  misc_feature 1..45: note = toxin sequence element
  source 1..45: mol_type = other DNA
aaacgcacctttaactattgcgcgtgggatggcagctttagcaaa

SEQ ID NO: 714 - DNA, 45 bp, synthetic construct
  misc_feature 1..45: note = toxin sequence element
  source 1..45: mol_type = other DNA
aaaagcaaatggccgcgcaacatttgcgtgtgggatggcagcgtg

SEQ ID NO: 715 - DNA, 33 bp, synthetic construct
  misc_feature 1..33: note = toxin sequence element
  source 1..33: mol_type = other DNA
aaaaccaccggcattgtgaaactgtgccgctgg

SEQ ID NO: 716 - DNA, 54 bp, synthetic construct
  misc_feature 1..54: note = toxin sequence element
  source 1..54: mol_type = other DNA
aaatgggtgttttttaccagcaaatttatgtgccgccgcgtgtggggcaaagat

SEQ ID NO: 717 - DNA, 54 bp, synthetic construct
  misc_feature 1..54: note = toxin sequence element
  source 1..54: mol_type = other DNA
ctgaaaccgaccctgcatggcatttggtataaacatcattattgctatacccag

SEQ ID NO: 718 - DNA, 54 bp, synthetic construct
  misc_feature 1..54: note = toxin sequence element
  source 1..54: mol_type = other DNA
ctgaaaccgaccctgcatggcatttggtataaaagctattattgctataaaaaa

SEQ ID NO: 719 - DNA, 27 bp, synthetic construct
  misc_feature 1..27: note = toxin sequence element
  source 1..27: mol_type = other DNA
aacggcaacaccgtgtatcgctgcgcg

SEQ ID NO: 720 - DNA, 39 bp, synthetic construct
  misc_feature 1..39: note = toxin sequence element
  source 1..39: mol_type = other DNA
aacaaaaaacatggctggtgcggctgggatggcaccttt

SEQ ID NO: 721 - DNA, 39 bp, synthetic construct
  misc_feature 1..39: note = toxin sequence element
  source 1..39: mol_type = other DNA
aacaaaaaatattggcattgcggctgggatggcaccttt

SEQ ID NO: 722 - DNA, 36 bp, synthetic construct
  misc_feature 1..36: note = toxin sequence element
  source 1..36: mol_type = other DNA
aaccgcaaacataaatggtgcaaatataaactgtgg

SEQ ID NO: 723 - DNA, 36 bp, synthetic construct
  misc_feature 1..36: note = toxin sequence element
  source 1..36: mol_type = other DNA
aaccgccgcgataaatggtgcaaatataaactgtgg

SEQ ID NO: 724 - DNA, 33 bp, synthetic construct
  misc_feature 1..33: note = toxin sequence element
  source 1..33: mol_type = other DNA
aacagccgcgataaatggtgcaaagtgctgctg

SEQ ID NO: 725 - DNA, 24 bp, synthetic construct
  misc_feature 1..24: note = toxin sequence element
  source 1..24: mol_type = other DNA
cagctgtggtgcaaaaaacgcctg

SEQ ID NO: 726 - DNA, 33 bp, synthetic construct
  misc_feature 1..33: note = toxin sequence element
  source 1..33: mol_type = other DNA
cagccggcgattaaatggtgcatttggagcccg

SEQ ID NO: 727 - DNA, 42 bp, synthetic construct
  misc_feature 1..42: note = toxin sequence element
  source 1..42: mol_type = other DNA
cagagccgcattgcgaacatgtggccgaccttttgcctggtg

SEQ ID NO: 728 - DNA, 42 bp, synthetic construct
  misc_feature 1..42: note = toxin sequence element
  source 1..42: mol_type = other DNA
cagagccgcattgcgaacatgtggccgaccttttgcagccag

SEQ ID NO: 729 - DNA, 42 bp, synthetic construct
  misc_feature 1..42: note = toxin sequence element
  source 1..42: mol_type = other DNA
cgcaaaaaatggccgtatcattgcggctgggatggcaccttt

SEQ ID NO: 730 - DNA, 42 bp, synthetic construct
  misc_feature 1..42: note = toxin sequence element
  source 1..42: mol_type = other DNA
cgcaaaaaatggccgtatcattgcgtgtgggattggaccgtg

SEQ ID NO: 731 - DNA, 24 bp, synthetic construct
  misc_feature 1..24: note = toxin sequence element
  source 1..24: mol_type = other DNA
cgcctgtggtgcaaaaaaattatt

SEQ ID NO: 732 - DNA, 33 bp, synthetic construct
  misc_feature 1..33: note = toxin sequence element
  source 1..33: mol_type = other DNA
cgcctgtggtgcaaaaaaaaaattgaagaaggc

SEQ ID NO: 733 - DNA, 30 bp, synthetic construct
  misc_feature 1..30: note = toxin sequence element
  source 1..30: mol_type = other DNA
cgcctgtggtgcaaaaaaaaaattgaatgg

SEQ ID NO: 734 - DNA, 27 bp, synthetic construct
  misc_feature 1..27: note = toxin sequence element
  source 1..27: mol_type = other DNA
cgcctgtggtgcaaaaaaaaactgtgg

SEQ ID NO: 735 - DNA, 24 bp, synthetic construct
  misc_feature 1..24: note = toxin sequence element
  source 1..24: mol_type = other DNA
cgcctgtggtgcaaaaaacgcctg

SEQ ID NO: 736 - DNA, 24 bp, synthetic construct
  misc_feature 1..24: note = toxin sequence element
  source 1..24: mol_type = other DNA
cgcctgtggtgcaaactggattgg

SEQ ID NO: 737 - DNA, 30 bp, synthetic construct
  misc_feature 1..30: note = toxin sequence element
  source 1..30: mol_type = other DNA
cgcctgtggtgcaaacgcattattaacatg

SEQ ID NO: 738 - DNA, 24 bp, synthetic construct
  misc_feature 1..24: note = toxin sequence element
  source 1..24: mol_type = other DNA
cgcctgtggtgcaaatataaactg

SEQ ID NO: 739 - DNA, 42 bp, synthetic construct
  misc_feature 1..42: note = toxin sequence element
  source 1..42: mol_type = other DNA
cgccgcgcgaaaccgagctggtgcggctgggattttaccttt

SEQ ID NO: 740 - DNA, 42 bp, synthetic construct
  misc_feature 1..42: note = toxin sequence element
  source 1..42: mol_type = other DNA
cgccgcgcgaaaccgagctggtgcggctgggattttaccgtg

SEQ ID NO: 741 - DNA, 45 bp, synthetic construct
  misc_feature 1..45: note = toxin sequence element
  source 1..45: mol_type = other DNA
cgccgcaccctgccgacctattgcgcgtgggatctgacctttccg

SEQ ID NO: 742 - DNA, 39 bp, synthetic construct
  misc_feature 1..39: note = toxin sequence element
  source 1..39: mol_type = other DNA
cgcagcgatggcaaatattgcgcgtgggatggcaccttt

SEQ ID NO: 743 - DNA, 42 bp, synthetic construct
  misc_feature 1..42: note = toxin sequence element
  source 1..42: mol_type = other DNA
cgcagcgattggaaatattgcgcgtgggatggcacctttagc

SEQ ID NO: 744 - DNA, 36 bp, synthetic construct
  misc_feature 1..36: note = toxin sequence element
  source 1..36: mol_type = other DNA
cgcagccgcgatcagtggtgcaaatataaactgtgg

SEQ ID NO: 745 - DNA, 36 bp, synthetic construct
  misc_feature 1..36: note = toxin sequence element
  source 1..36: mol_type = other DNA
cgcgtgcgcgatcagtggtgcaaatataaactgtgg

SEQ ID NO: 746 - DNA, 33 bp, synthetic construct
  misc_feature 1..33: note = toxin sequence element
  source 1..33: mol_type = other DNA
agcgataaacataaatggtgcaaatggaaactg

SEQ ID NO: 747 - DNA, 18 bp, synthetic construct
  misc_feature 1..18: note = toxin sequence element
  source 1..18: mol_type = other DNA
agccataacaaatgcacc

SEQ ID NO: 748 - DNA, 33 bp, synthetic construct
  misc_feature 1..33: note = toxin sequence element
  source 1..33: mol_type = other DNA
agcaaactgtttaaactgtgcaactttagcttt

SEQ ID NO: 749 - DNA, 42 bp, synthetic construct
  misc_feature 1..42: note = toxin sequence element
  source 1..42: mol_type = other DNA
agcaaaaccggctttgtgaaaaacatttgcaaatatgaaatg

SEQ ID NO: 750 - DNA, 36 bp, synthetic construct
  misc_feature 1..36: note = toxin sequence element
  source 1..36: mol_type = other DNA
agcaaaacctggggctggtgcgcggtggaagcgccg

SEQ ID NO: 751 - DNA, 45 bp, synthetic construct
  misc_feature 1..45: note = toxin sequence element
  source 1..45: mol_type = other DNA
agcccgaaacatggctggtgcgtgtgggattggacctttcgcaaa

SEQ ID NO: 752 - DNA, 36 bp, synthetic construct
  misc_feature 1..36: note = toxin sequence element
  source 1..36: mol_type = other DNA
agcccgaaatggggcctgtgcaactttccgatgccg

SEQ ID NO: 753 - DNA, 45 bp, synthetic construct
  misc_feature 1..45: note = toxin sequence element
  source 1..45: mol_type = other DNA
agcccgcgctggggctggtgcatttatagcacccgcggcggccgc

SEQ ID NO: 754 - DNA, 45 bp, synthetic construct
  misc_feature 1..45: note = toxin sequence element
  source 1..45: mol_type = other DNA
agcccgacctggaaatggtgcgtgctgaaaagcccgggccgccgc

SEQ ID NO: 755 - DNA, 36 bp, synthetic construct
  misc_feature 1..36: note = toxin sequence element
  source 1..36: mol_type = other DNA
agcccgacctggaaatggtgcgtgtatgcgcgcccg

SEQ ID NO: 756 - DNA, 30 bp, synthetic construct
  misc_feature 1..30: note = toxin sequence element
  source 1..30: mol_type = other DNA
agccagcatcgcctgtgcagcgtgaaagcg

SEQ ID NO: 757 - DNA, 33 bp, synthetic construct
  misc_feature 1..33: note = toxin sequence element
  source 1..33: mol_type = other DNA
agccgcaaagataaatggtgcaaatatcagatt

SEQ ID NO: 758 - DNA, 33 bp, synthetic construct
  misc_feature 1..33: note = toxin sequence element
  source 1..33: mol_type = other DNA
agccgcaaacatcgctggtgcaaatatgaaatt

SEQ ID NO: 759 - DNA, 33 bp, synthetic construct
  misc_feature 1..33: note = toxin sequence element
  source 1..33: mol_type = other DNA
agccgcaaaacccgctggtgcaaatatcagatt

SEQ ID NO: 760 - DNA, 33 bp, synthetic construct
  misc_feature 1..33: note = toxin sequence element
  source 1..33: mol_type = other DNA
agccgccagctgtgcaaatatgtgattgattgg

SEQ ID NO: 761 - DNA, 33 bp, synthetic construct
  misc_feature 1..33: note = toxin sequence element
  source 1..33: mol_type = other DNA
agccgccgcgatcgctggtgcaaatatgatctg

SEQ ID NO: 762 - DNA, 33 bp, synthetic construct
  misc_feature 1..33: note = toxin sequence element
  source 1..33: mol_type = other DNA
agccgccgcgatcgctggtgcaaatattatctg

SEQ ID NO: 763 - DNA, 36 bp, synthetic construct
  misc_feature 1..36: note = toxin sequence element
  source 1..36: mol_type = other DNA
agccgccgcggcaccaacccggaaaaacgctgccgc

SEQ ID NO: 764 - DNA, 42 bp, synthetic construct
  misc_feature 1..42: note = toxin sequence element
  source 1..42: mol_type = other DNA
agccgccgccatggctggtgcgtgtgggatggcacctttagc

SEQ ID NO: 765 - DNA, 39 bp, synthetic construct
  misc_feature 1..39: note = toxin sequence element
  source 1..39: mol_type = other DNA
agccgcacctggaaatggtgcgtgctggcgggcccgtgg

SEQ ID NO: 766 - DNA, 33 bp, synthetic construct
  misc_feature 1..33: note = toxin sequence element
  source 1..33: mol_type = other DNA
agcagcaaacataaatggtgcaaagtgtatctg

SEQ ID NO: 767 - DNA, 39 bp, synthetic construct
  misc_feature 1..39: note = toxin sequence element
  source 1..39: mol_type = other DNA
agcagccgctggaaatggtgcgtgctggcgagcccgttt

SEQ ID NO: 768 - DNA, 39 bp, synthetic construct
  misc_feature 1..39: note = toxin sequence element
  source 1..39: mol_type = other DNA
agcagccgctggaaatggtgcgtgctgccggcgccgtgg

SEQ ID NO: 769 - DNA, 45 bp, synthetic construct
  misc_feature 1..45: note = toxin sequence element
  source 1..45: mol_type = other DNA
acctttaaagaaaacgaaaacggcaacaccgtgaaacgctgcgat

SEQ ID NO: 770 - DNA, 45 bp, synthetic construct
  misc_feature 1..45: note = toxin sequence element
  source 1..45: mol_type = other DNA
acctttaaaaccaacgaaaacggcaacaccgtgaaacgctgcgat

SEQ ID NO: 771 - DNA, 21 bp, synthetic construct
  misc_feature 1..21: note = toxin sequence element
  source 1..21: mol_type = other DNA
accggcctgtgcattccgccg

SEQ ID NO: 772 - DNA, 24 bp, synthetic construct
  misc_feature 1..24: note = toxin sequence element
  source 1..24: mol_type = other DNA
acccgctttaacgtgtgcggcaaa

SEQ ID NO: 773 - DNA, 27 bp, synthetic construct
  misc_feature 1..27: note = toxin sequence element
  source 1..27: mol_type = other DNA
acctggccgaccgaaatttgcattgat

SEQ ID NO: 774 - DNA, 45 bp, synthetic construct
  misc_feature 1..45: note = toxin sequence element
  source 1..45: mol_type = other DNA
acctataaagcgaacgaaaacggcaaccaggtgaaacgctgcgat

SEQ ID NO: 775 - DNA, 45 bp, synthetic construct
  misc_feature 1..45: note = toxin sequence element
  source 1..45: mol_type = other DNA
acctataaagaaaacgaaaacggcaacaccgtgaaacgctgcgat

SEQ ID NO: 776 - DNA, 45 bp, synthetic construct
  misc_feature 1..45: note = toxin sequence element
  source 1..45: mol_type = other DNA
acctataaagaaaacgaaaacggcaacaccgtgcagcgctgcgat

SEQ ID NO: 777 - DNA, 45 bp, synthetic construct
  misc_feature 1..45: note = toxin sequence element
  source 1..45: mol_type = other DNA
acctataaaaccaacgaaaacggcaacagcgtgcagcgctgcgat

SEQ ID NO: 778 - DNA, 54 bp, synthetic construct
  misc_feature 1..54: note = toxin sequence element
  source 1..54: mol_type = other DNA
gtgaaaaccagcggctattggtggtataaaaaaacctattgccgccgcaaaagc

SEQ ID NO: 779 - DNA, 51 bp, synthetic construct
  misc_feature 1..51: note = toxin sequence element
  source 1..51: mol_type = other DNA
tggaaacgccgccgcagctttgaagtgtgcgtgccgaaaaccccgaaaacc

SEQ ID NO: 780 - DNA, 18 bp, synthetic construct
  misc_feature 1..18: note = toxin sequence element
  source 1..18: mol_type = other DNA
ctgggcgtgtgcatgtgg

SEQ ID NO: 781 - DNA, 15 bp, synthetic construct
  misc_feature 1..15: note = toxin sequence element
  source 1..15: mol_type = other DNA
ttttttgcgtgcgcg

SEQ ID NO: 782 - DNA, 15 bp, synthetic construct
  misc_feature 1..15: note = toxin sequence element
  source 1..15: mol_type = other DNA
gtgctgttttgcgtg

SEQ ID NO: 783 - DNA, 18 bp, synthetic construct
  misc_feature 1..18: note = toxin sequence element
  source 1..18: mol_type = other DNA
attgtgtttgtgtgcacc

SEQ ID NO: 784 - DNA, 18 bp, synthetic construct
  misc_feature 1..18: note = toxin sequence element
  source 1..18: mol_type = other DNA
attctgctgttttgcagc

SEQ ID NO: 785 - DNA, 15 bp, synthetic construct
  misc_feature 1..15: note = toxin sequence element
  source 1..15: mol_type = other DNA
gtgtttgtgtgcatt

SEQ ID NO: 786 - DNA, 18 bp, synthetic construct
  misc_feature 1..18: note = toxin sequence element
  source 1..18: mol_type = other DNA
gcgcagtttatttgcctg

SEQ ID NO: 787 - DNA, 18 bp, synthetic construct
  misc_feature 1..18: note = toxin sequence element
  source 1..18: mol_type = other DNA
ggcccgtttgtgtgcgtg

SEQ ID NO: 788 - protein, 83 aa, synthetic construct
  REGION 1..83: note = toxin sequence element
ECRYLFGGCKTTSDCCKHLGCKFRDKYCAWDFTFSGNGNGNGSSTCIPSGQPCADSDDCCETFHCKWVFFTSKFMCRRVWGKD

SEQ ID NO: 789 - protein, 92 aa, synthetic construct
  REGION 1..92: note = toxin sequence element
SSTCIPSGQPCADSDDCCETFHCKWVFFTSKFMCRRVWGKDDSSPYVPVTTSSTCIPSGQPCADSDDCCETFHCKWVFFTSKFMCRRVWGKD

SEQ ID NO: 790 - protein, 92 aa, synthetic construct
  REGION 1..92: note = toxin sequence element
SSTCIPSGQPCADSDDCCETFHCKWVFFTSKFMCRRVWGKDDSSGNGNGNGSSTCIPSGQPCADSDDCCETFHCKWVFFTSKFMCRRVWGKD

SEQ ID NO: 791 - protein, 120 aa, synthetic construct
  REGION 1..120: note = toxin sequence element
SSTCIPSGQPCADSDDCCETFHCKWVFFTSKFMCRRVWGKDDSSGGNGNGNGNGNGNGNGAAAGGNGNGNGNGNGNGNGSSTCIPSGQPCADSDDCCETFHCKWVFFTSKFMCRRVWGKD

SEQ ID NO: 792 - protein, 56 aa, synthetic construct
  REGION 1..56: note = toxin sequence element
MSALLILALVGAAVASSTCIPSGQPCADSDDCCETFHCKWVFFTSKFMCRRVWGKD

SEQ ID NO: 793 - protein, 98 aa, synthetic construct
  REGION 1..98: note = toxin sequence element
MSALLILALVGAAVAECRYLFGGCKTTSDCCKHLGCKFRDKYCAWDFTFSGNGNGNGSSTCIPSGQPCADSDDCCETFHCKWVFFTSKFMCRRVWGKD

SEQ ID NO: 794 - protein, 107 aa, synthetic construct
  REGION 1..107: note = toxin sequence element
MSALLILALVGAAVASSTCIPSGQPCADSDDCCETFHCKWVFFTSKFMCRRVWGKDDSSPYVPVTTSSTCIPSGQPCADSDDCCETFHCKWVFFTSKFMCRRVWGKD

SEQ ID NO: 795 - protein, 107 aa, synthetic construct
  REGION 1..107: note = toxin sequence element
MSALLILALVGAAVASSTCIPSGQPCADSDDCCETFHCKWVFFTSKFMCRRVWGKDDSSGNGNGNGSSTCIPSGQPCADSDDCCETFHCKWVFFTSKFMCRRVWGKD

SEQ ID NO: 796 - protein, 135 aa, synthetic construct
  REGION 1..135: note = toxin sequence element
MSALLILALVGAAVASSTCIPSGQPCADSDDCCETFHCKWVFFTSKFMCRRVWGKDDSSGGNGNGNGNGNGNGNGAAAGGNGNGNGNGNGNGNGSSTCIPSGQPCADSDDCCETFHCKWVFFTSKFMCRRVWGKD

SEQ ID NO: 797 - protein, 67 aa, synthetic construct
  REGION 1..67: note = toxin sequence element
MSALLILALVGAAVASSTCIPSGQPCADSDDCCETFHCKWVFFTSKFMCRRVWGKDGEQKLISEEDL

SEQ ID NO: 798 - protein, 109 aa, synthetic construct
  REGION 1..109: note = toxin sequence element
MSALLILALVGAAVAECRYLFGGCKTTSDCCKHLGCKFRDKYCAWDFTFSGNGNGNGSSTCIPSGQPCADSDDCCETFHCKWVFFTSKFMCRRVWGKDGEQKLISEEDL

SEQ ID NO: 799 - protein, 118 aa, synthetic construct
  REGION 1..118: note = toxin sequence element
MSALLILALVGAAVASSTCIPSGQPCADSDDCCETFHCKWVFFTSKFMCRRVWGKDDSSPYVPVTTSSTCIPSGQPCADSDDCCETFHCKWVFFTSKFMCRRVWGKDGEQKLISEEDL

SEQ ID NO: 800 - protein, 118 aa, synthetic construct
  REGION 1..118: note = toxin sequence element
MSALLILALVGAAVASSTCIPSGQPCADSDDCCETFHCKWVFFTSKFMCRRVWGKDDSSGNGNGNGSSTCIPSGQPCADSDDCCETFHCKWVFFTSKFMCRRVWGKDGEQKLISEEDL

SEQ ID NO: 801 - protein, 146 aa, synthetic construct
  REGION 1..146: note = toxin sequence element
MSALLILALVGAAVASSTCIPSGQPCADSDDCCETFHCKWVFFTSKFMCRRVWGKDDSSGGNGNGNGNGNGNGNGAAAGGNGNGNGNGNGNGNGSSTCIPSGQPCADSDDCCETFHCKWVFFTSKFMCRRVWGKDGEQKLISEEDL

SEQ ID NO: 802 - protein, 96 aa, synthetic construct
  REGION 1..96: note = toxin sequence element
MSALLILALVGAAVASSTCIPSGQPCADSDDCCETFHCKWVFFTSKFMCRRVWGKDGEQKLISEEDLGALCNGAGFATPVTLALVPALLATFWSLL

SEQ ID NO: 803 - protein, 138 aa, synthetic construct
  REGION 1..138: note = toxin sequence element
MSALLILALVGAAVAECRYLFGGCKTTSDCCKHLGCKFRDKYCAWDFTFSGNGNGNGSSTCIPSGQPCADSDDCCETFHCKWVFFTSKFMCRRVWGKDGEQKLISEEDLGALCNGAGFATPVTLALVPALLATFWSLL

SEQ ID NO: 804 - protein, 147 aa, synthetic construct
  REGION 1..147: note = toxin sequence element
MSALLILALVGAAVASSTCIPSGQPCADSDDCCETFHCKWVFFTSKFMCRRVWGKDDSSPYVPVTTSSTCIPSGQPCADSDDCCETFHCKWVFFTSKFMCRRVWGKDGEQKLISEEDLGALCNGAGFATPVTLALVPALLATFWSLL

SEQ ID NO: 805 - protein, 147 aa, synthetic construct
  REGION 1..147: note = toxin sequence element
MSALLILALVGAAVASSTCIPSGQPCADSDDCCETFHCKWVFFTSKFMCRRVWGKDDSSGNGNGNGSSTCIPSGQPCADSDDCCETFHCKWVFFTSKFMCRRVWGKDGEQKLISEEDLGALCNGAGFATPVTLALVPALLATFWSLL

SEQ ID NO: 806 - protein, 175 aa, synthetic construct
  REGION 1..175: note = toxin sequence element
MSALLILALVGAAVASSTCIPSGQPCADSDDCCETFHCKWVFFTSKFMCRRVWGKDDSSGGNGNGNGNGNGNGNGAAAGGNGNGNGNGNGNGNGSSTCIPSGQPCADSDDCCETFHCKWVFFTSKFMCRRVWGKDGEQKLISEEDLGALCNGAGFATPVTLALVPALLATFWSLL

SEQ ID NO: 807 - DNA, 1167 bp, synthetic construct
  misc_feature 1..1167: note = Transmembrane T-toxin
  source 1..1167: mol_type = other DNA
atgtctgcacttctgatcctagctcttgttggagctgcaatgagcagcacctgcattccgagcggccagccgtgtgcggatagcgatgattgctgcgaaacctttcattgcaaatgggtgttttttaccagcaaatttatgtgccgccgcgtgtggggcaaagataccggtgttgctatcgattacaaagacgatgacgacaagcttgcggccgctggtaacggaaatggcaacgggaatggcaacggtaacggaaacggggatggtactcgagttgctgtgggccaggacacgcaggaggtcatcgtggtgccacactccttgccctttaaggtggtggtgatctcagccatcctggccctggtggtgctcaccatcatctcccttatcatcctcatcatgctttggcagaagaaaccacgtaggatccaccggccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggccttcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaaactgagcaaagaccccaacgagaagcgcgatcacatggtcctgctggagttcgtgaccgccgccgggatcactctcggcatggacgagctgtacaagtaa

SEQ ID NO: 808 - protein, 388 aa, synthetic construct
  REGION 1..388: note = Transmembrane T-toxin
MSALLILALVGAAMSSTCIPSGQPCADSDDCCETFHCKWVFFTSKFMCRRVWGKDTGVAIDYKDDDDKLAAAGNGNGNGNGNGNGNGDGTRVAVGQDTQEVIVVPHSLPFKVVVISAILALVVLTIISLIILIMLWQKKPRRIHRPVATMVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSKLSKDPNEKRDHMVLLEFVTAAGITLGMDELYK

SEQ ID NO: 809 - protein, 9 aa, synthetic construct
  REGION 1..9: note = toxin sequence element
GCKWYLGDC

SEQ ID NO: 810 - protein, 38 aa, synthetic construct
  REGION 1..38: note = toxin sequence element
GVEINVKCSGSPQCLKPCKDAGMDFGDCMNDKCHCTPK

SEQ ID NO: 811 - protein, 36 aa, synthetic construct
  REGION 1..36: note = chimeric Hv1
VVVVISFILDIVLLFQEHQFEALGLLILLRLWRVFR

SEQ ID NO: 812 - protein, 38 aa, synthetic construct
  REGION 1..38: note = chimeric Hv1
VVVVVSFGVDIALIFVGESEALAAIGLLVILRLWRVAR

SEQ ID NO: 813 - protein, 36 aa, synthetic construct
  REGION 1..36: note = chimeric Hv1
VVVVISFGVDIALLFQEHQFEALGLLILLRLWRVFR

SEQ ID NO: 814 - protein, 36 aa, synthetic construct
  REGION 1..36: note = chimeric Hv1
VVVVISFGVDIALLFQEHQFAAIGLLVILRLWRVFR

SEQ ID NO: 815 - protein, 36 aa, synthetic construct
  REGION 1..36: note = chimeric Hv1
VVVVISFILDIVLLFQEHQFAAIGLLVILRLWRVFR

SEQ ID NO: 816 - protein, 38 aa, synthetic construct
  REGION 1..38: note = chimeric Hv1
VVVVVSFILDIVLLFVGESEALAAIGLLVILRLWRVAR

SEQ ID NO: 817 - protein, 36 aa, synthetic construct
  REGION 1..36: note = chimeric Hv1
VVVVISFGVDIALIFQEHQFEALGLLILLRLWRVFR

SEQ ID NO: 818 - protein, 36 aa, synthetic construct; identical to SEQ ID NO: 817
  REGION 1..36: note = chimeric Hv1

SEQ ID NO: 819 - protein, 36 aa, synthetic construct; identical to SEQ ID NO: 811
  REGION 1..36: note = chimeric Hv1

SEQ ID NO: 820 - protein, 36 aa, synthetic construct
  REGION 1..36: note = chimeric Hv1
VVVVISFGVDIALLFQEHQFEALGLLVILRLWRVFR

SEQ ID NO: 821 - protein, 36 aa, synthetic construct
  REGION 1..36: note = chimeric Hv1
VVVVISFILDIVLLFQEHQFEALGLLVILRLWRVFR

(19) United States
(12) Patent Application Publication
(10) Pub. No.: US 2025/0115647 A1
(43) Pub. Date: Apr. 10, 2025

(54) HV1 MODULATORS AND USES

(71) Applicant: The Regents of the University of California, Oakland, CA (US)

(72) Inventor: Steven Alan GOLDSTEIN, Chicago, IL (US)

(21) Appl. No.: 18/815,668

(22) Filed: Aug. 26, 2024

Related U.S. Application Data
(63) Continuation of application No. 17/684,346, filed on Mar. 1, 2022, now Pat. No. 12,084,481.
Application No. 17/684,346 is a continuation of application No. 16/474,906, filed on Jun. 28, 2019, now Pat. No. 11,274,130.
(60) Provisional application No. 62/441,097, filed on Dec. 30, 2016.
(60) Provisional application No. 62/447,433, filed on Jan. 17, 2017.

(51) Int. Cl. C07K 14/435; C07K 14/705

(52) U.S. Cl. CPC C07K 14/43518; C07K 14/43522; C07K 14/705; C07K 2319/02; C07K 2319/41

(57) ABSTRACT

The present invention provides novel agents for modulation of Hv1 channels. The present invention provides agents for activating and/or inhibiting Hv1 channel function and/or activity, and reagents and methods relating thereto.

### RELATED APPLICATIONS

This application is a continuation of U.S. patent application Ser. No. (USSN) 17/684,346, Mar. 1, 2022, now pending, which is a continuation of U.S. Ser. No. 16/474,906, filed Jun. 28, 2019, now USPN 11, 274,130, issue date Mar. 15, 2022, which is a National Phase Patent Application claiming benefit of priority under 35 U.S.C. § 371 to International (PCT) Patent Application serial number PCT/US2017/068896, filed Dec. 29, 2017, now expired, which claims benefit to U.S. Provisional Application No. 62/441,097, filed Dec. 30, 2016, now expired and U.S. Provisional Application No. 62/447,433, filed Jan. 17, 2017, now expired. The aforementioned applications are expressly incorporated herein by reference in their entirety and for all purposes.

### STATEMENT AS TO FEDERALLY SPONSORED RESEARCH

This invention was made with government support under ROIGM111716 awarded by the National Institutes of Health (NIH). The government has certain rights in the invention.

### REFERENCE TO ELECTRONIC SEQUENCE LISTING

The application contains a Sequence Listing which has been submitted electronically in .XML format and is hereby incorporated by reference in its entirety. Said .XML copy, created on Aug. 23, 2024, is named “5825_137088C1.xml” and is 1,008,965 bytes in size. The sequence listing contained in this .XML file is part of the specification and is hereby incorporated by reference herein in its entirety.

### BACKGROUND

Voltage-gated ion channels facilitate the transfer of ions across cell membranes and function as key components of essential cellular processes. One particular type of voltage-gated ion channel is the voltage-gated proton channel (Hv1). Hv1 is a transmembrane protein that regulates the transfer of protons across cell membranes. When the Hv1 channel is open, protons permeate the channel and cross the cellular membrane.

The Hv1 channel is expressed in many different tissues and is associated with a wide variety of physiological and pathological processes. For example, Hv1 channels may play a role in immune defense, sperm activity, and cancer progression. For these and other reasons, Hv1 may be an attractive drug target (Seredenina, T., et al. “Voltage-gated proton channels as novel drug targets: from NADPH oxidase regulation to sperm biology.” Antioxid Redox Signal. 10; 23(5): 490-513 (2015)). However, clinically compatible Hv1 activators or inhibitors are not known. For these reasons, there is a need for the development of activators and inhibitors of Hv1 channels.

### SUMMARY

The present disclosure provides technologies relating to modulation of Hv1 channels. Among other things, the present disclosure provides Hv1 modulating agents, and various compositions and methods relating thereto.

In some embodiments, an Hv1 modulating agent is or comprises an engineered polypeptide component having an inhibitor cysteine knot (ICK)-like structural motif.

In some embodiments, an Hv1 modulating agent is or comprises an engineered polypeptide component that includes one or more toxin sequence elements, each of which has an amino acid sequence that is substantially identical to, but differs from, that of a corresponding element found in a wild-type toxin.

In some embodiments, an Hv1 modulating agent shares one or more cysteines with a wild-type toxin sequence. In some embodiments, an Hv1 modulating agent shares the same approximate relative position of cysteines with a wild-type toxin.

In some embodiments, an Hv1 modulating agent is or comprises a polypeptide sequence set forth in Tables 2A, 3A, and 4.

In some embodiments, an Hv1 modulating agent is encoded by a nucleotide sequence that is or comprises a sequence set forth in Tables 2C and 3B.

In some embodiments, an Hv1 modulating agent can be expressed from a vector including a nucleic acid sequence encoding the Hv1 modulating agent.

In some embodiments, an Hv1 modulating agent binds to the external surface of human Hv1. In some embodiments, an Hv1 modulating agent binds to the S3-S4 external loop region of human Hv1.

In some embodiments, an Hv1 modulating agent inhibits human Hv1 function. For example, in some embodiments, an Hv1 modulating agent may decrease or block proton current. In some embodiments, an Hv1 modulating agent may reduce the number or likelihood of Hv1 channel opening. In some embodiments, an Hv1 modulating agent may increase the rate of Hv1 channel closing.

In some embodiments, an Hv1 modulating agent activates human Hv1 function. For example, in some embodiments, an Hv1 modulating agent increases proton current. In some embodiments, an Hv1 modulating agent increase the rate of Hv1 channel opening. In some embodiments, an Hv1 modulating agent slows the rate of Hv1 channel closing.

In some embodiments, an Hv1 modulating agent inhibits sperm capacitation.

In some embodiments, an Hv1 modulating agent decreases reactive oxygen species (ROS) production in white blood cells.

The present invention further provides various reagents and methods associated with Hv1 modulating agents including, for example, systems for identifying and characterizing them, strategies for preparing them, and various therapeutic compositions and methods relating to them. Further description of certain embodiments of these aspects, and others, of the present invention, is presented below.

### BRIEF DESCRIPTION OF THE DRAWING

FIG. 1 Presents an amino acid sequence alignment of exemplary Hv1 modulating agents C5 and C6. Six conserved cysteine residues and three disulfide bridges of an inhibitor cysteine knot (ICK)-like structural motif are indicated.

FIG. 2 Presents the amino acid sequences of exemplary Hv1 modulating agents C5 and C6 and the amino acid sequence of hanatoxin (HaTx1). Sequence elements corresponding to sequences in Table 3A are labeled. Conserved cysteine residues are highlighted. NT, N-terminus. CT, C-terminus.

FIG. 3A-F: Presents exemplary T-toxin amino acid sequences and effects of T-toxins on hHv1 function:

FIG. 3A depicts an exemplary T-toxin comprising the amino acid sequence of Hv1 modulating agent C6 linked to a trypsin secretory signal sequence at the N-terminus, a 16 amino acid linker with embedded C-Myc epitope tag at the C-terminus, and a hydrophobic sequence for GPI attachment from the mammalian Lynx1 peptide;

FIG. 3B depicts an exemplary T-toxin comprising an N-terminal trypsin secretory signal sequence, the amino acid sequence of HaTx1 linked by a flexible 7 amino acid linker to C6, a C-Myc epitope embedded into a 6 amino acid linker at the C-terminus, and a hydrophobic sequence for GPI attachment;

FIG. 3C depicts an exemplary T-toxin comprising an N-terminal trypsin secretory signal sequence, C6 dimers linked by a rigid 10 amino acid linker, a C-Myc epitope embedded into a 6 amino acid linker at the C-terminus, and a hydrophobic sequence for GPI attachment;

FIG. 3D depicts an exemplary T-toxin comprising an N-terminal trypsin secretory signal sequence, C6 dimers linked by a flexible 10 amino acid linker, a C-Myc epitope embedded into a 6 amino acid linker at the C-terminus, and a hydrophobic sequence for GPI attachment;

FIG. 3E depicts an exemplary T-toxin comprising an N-terminal trypsin secretory signal sequence, C6 dimers linked by long flexible 38 amino acid linker, a C-Myc epitope embedded into a 6 amino acid linker at the C-terminus, and a hydrophobic sequence for GPI attachment; and

FIG. 3F shows inhibition of wild-type hHv1 measured as unblocked fractional current in oocytes expressing only hHv1 or both hHv1 and T-toxin.

FIG. 4A-B: Illustrates activating and inhibiting effects of exemplary Hv1 modulators C5 and C6 on hHv1. Whole-cell patch clamp recordings were performed on HEK-293T cells over-expressing hHv1:

FIG. 4A graphically illustrates proton currents with C5 (FIG. 4A); and

FIG. 4B graphically illustrates proton currents with C6 (FIG. 4B),
each figure illustrating current with and without any peptide or modifications (black traces).

FIG. 5A-K: Illustrates that Hv1 modulating agent C6 affects response of sperm to progesterone, but not other changes related to sperm capacitation:

FIG. 5A graphically illustrates that C6 did not affect the vitality (FIG. 5A),

FIG. 5F graphically illustrates that C6 did not affect the protein tyrosine phosphorylation (FIG. 5F and FIG. 5G),

FIG. 5G graphically illustrates that C6 did not affect the protein tyrosine phosphorylation,

FIG. 5H graphically illustrates that C6 did not affect the cholesterol content of the membranes (FIG. 5H);

FIG. 5B illustrates an image showing that C6 did not significantly alter the mobility of sperm;

FIG. 5C graphically illustrates that C6 did not significantly alter the mobility of sperm to VSL;

FIG. 5D graphically illustrates that C6 did not significantly alter the mobility of sperm to progesterone; and
to progesterone that C6 did not significantly alter the mobility of sperm to VAP

FIG. 5I graphically illustrates the (F/Fo)⁻¹ as a function of time showing that the increase of cytosolic calcium triggered by progesterone is diminished in the presence of the Hv1 modulating agent and the acrosome reaction induced by the hormone is inhibited;

FIG. 5J graphically illustrates the d(F/Fo)⁻¹ as a function of time showing that the increase of cytosolic calcium triggered by progesterone is diminished in the presence of the Hv1 modulating agent and the acrosome reaction induced by the hormone is inhibited;

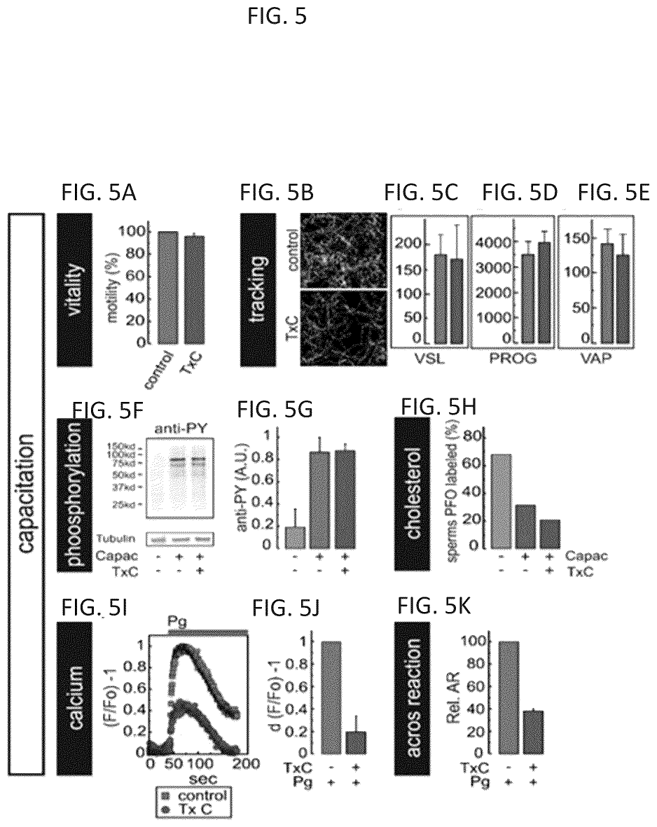

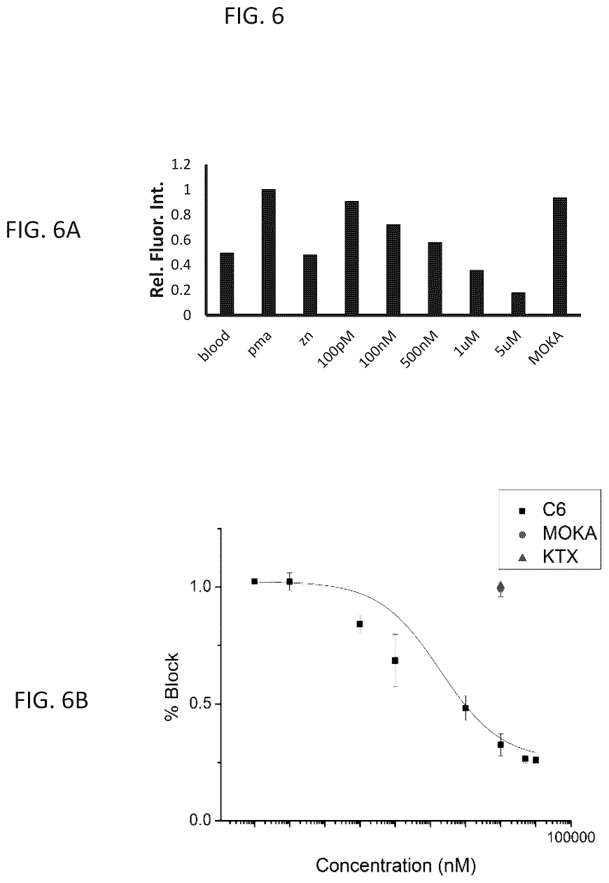

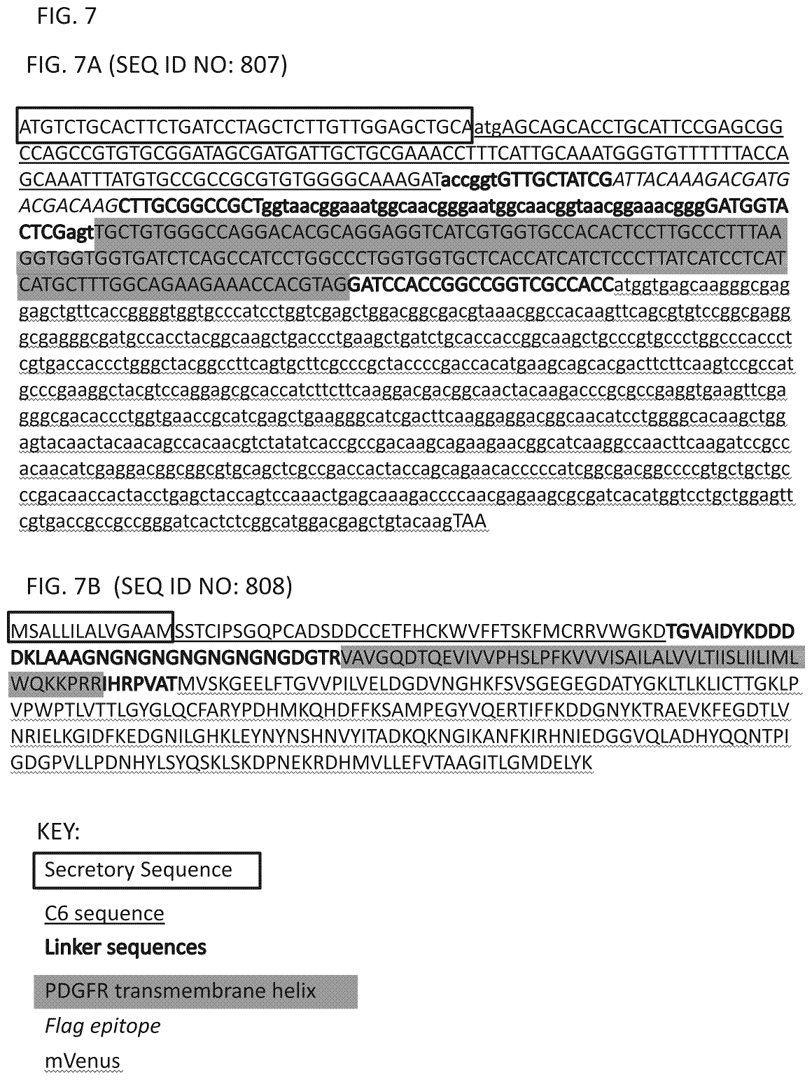

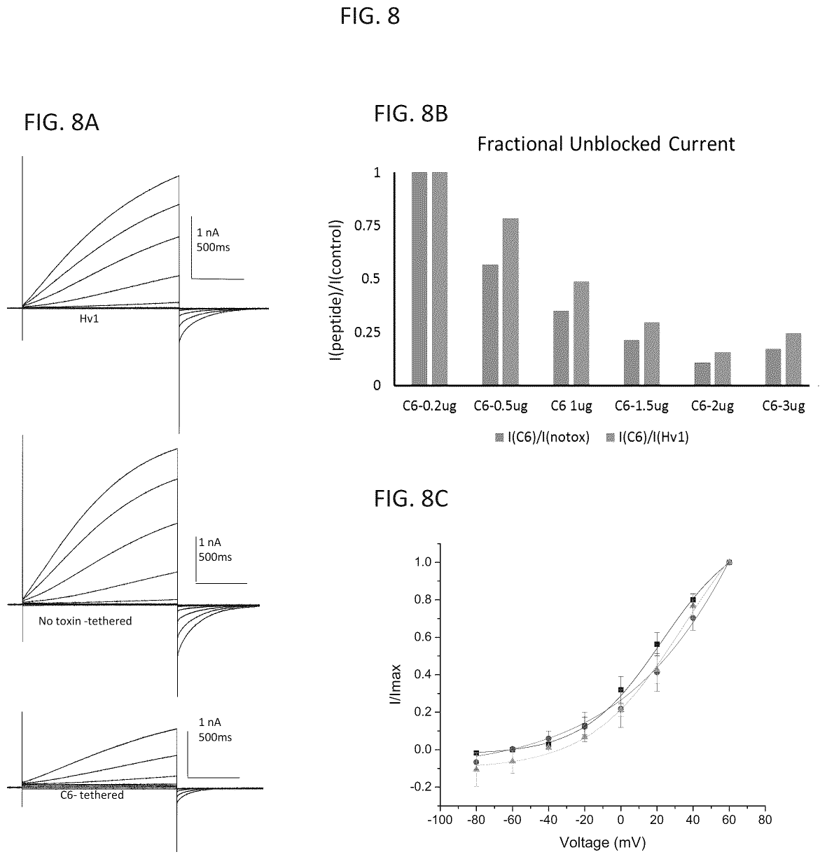

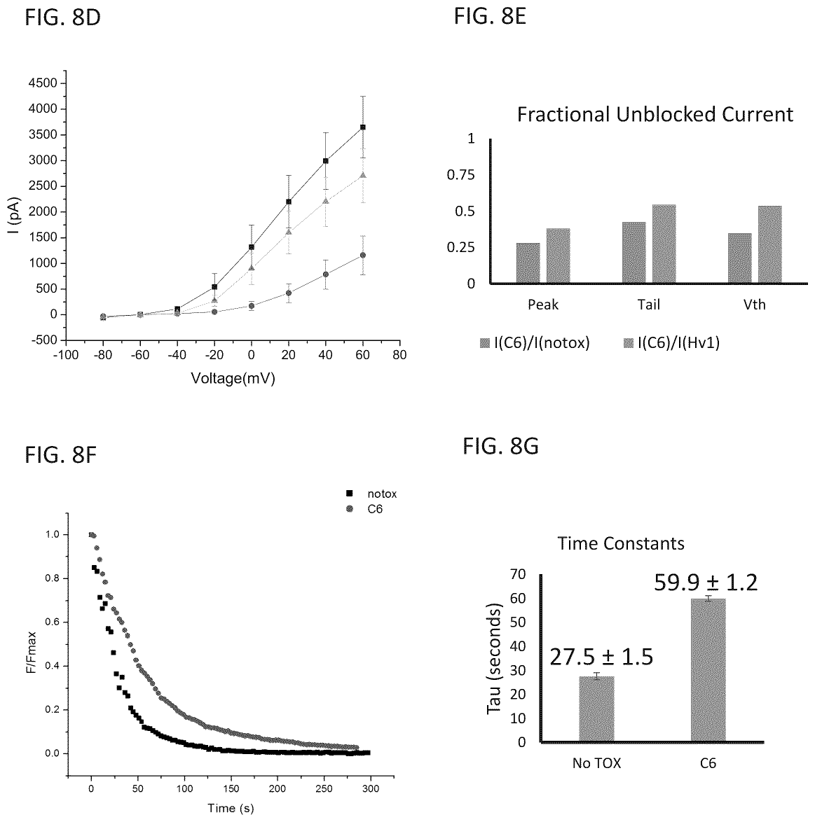

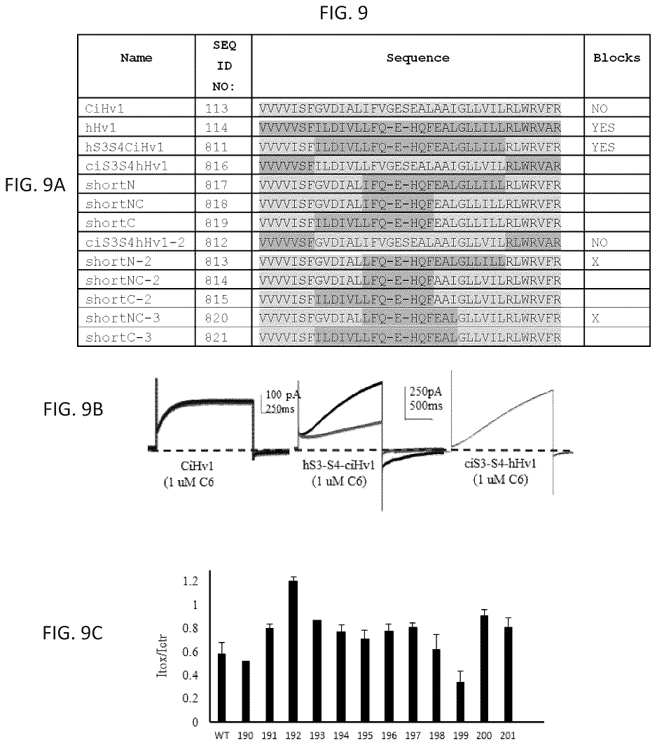

FIG. 5K graphically illustrates the across reaction showing that the increase of cytosolic calcium triggered by progesterone is diminished in the presence of the Hv1 modulating agent and the acrosome reaction induced by the hormone is inhibited;
and for FIGS. 5I-5K all responses with C6 were compared to control peptide; VSL, velocity straight line; PROG, progression; VAP, velocity average path; Capac, capacitating medium (Human tubal fluid (HTF) media supplemented with 5 mg/mL BSA; as described, for example, in Pocognoni, C. A., et al., “PerfringolysinO as a useful tool to study human sperm physiology,” Fertility & Sterility 99(1): 99-106(2013)).

FIG. 6A-B: Demonstrates that Hv1 modulating agent C6 blocks production of ROS in human blood cells in a dose-dependent manner; Phorbol myristate acetate (PMA) was used to stimulate ROS production as shown by the increase of fluorescence over baseline (blood); The known inhibitor of Hv1, zinc (Zn), blocks to background levels; Various concentrations of C6 also blocked fluorescence intensity in a dose-dependent manner; Two other toxins that block potassium channels with nM affinity (Moka and KTX) had no effect:

FIG. 6A graphically shows relative fluorescence intensity, measured at 590 nM (excited at 530 nM) using Amplex Red, which reacts with ROS to give a fluorescent product, for whole blood alone, PMA-stimulated whole blood, and PMA-stimulated whole blood with various inhibitors. 10 μM of MOKA toxin was used as a control; and

FIG. 6B graphically shows dose response curve plotted as the percentage of fluorescence from PMA-stimulated whole blood blocked versus concentration of C6 present. MOKA toxin and KTX were used as controls and showed no effect at 10 μM.

FIG. 7A-B FIG. 7A Presents an exemplary T-toxin nucleotide (SEQ ID NO: 807) sequence encoding a protein comprising a PDGFR transmembrane helix which links an internal mVenus fluorescent protein to an external C6.

FIG. 7B Presents an exemplary T-toxin amino acid (SEQ ID NO: 808) sequence comprising a PDGFR transmembrane helix which links an internal mVenus fluorescent protein to an external C6.

FIG. 8A-G: Illustrates effects of Hv1 modulating agent C6 tethered to HEK-293T cell surfaces via a PDGFR transmembrane link;

FIG. 8A graphically shows current recordings for WT Hv1 without any tether or peptide (top), expressed with the transmembrane mVenus without a peptide sequence (middle), or expressed with tethered mVenus-C6 (bottom);

FIG. 8B shows WT Hv1 current with various amounts of tethered toxin plasmid normalized to current with the transmembrane tether without peptide (I(C6)/I(notox)) or to WT Hv1 with no tether expressed (I(C6)/I(Hv1);

FIG. 8C demonstrates small shifts observed in g-V normalized to the maximum seen for each condition. Black line is WT Hv1, Green line is WT Hv1 with transmembrane without tether, Red line is WT-Hv1 with tethered C6;

FIG. 8D demonstrates current-voltage (I-V) showing the decrease in current in WT (black), WT with tether without toxin (green) or WT with tethered C6 (red);

FIG. 8E shows amount of current blocked by 1 g of expressed tethered C6 in peak (end of pulse) or tail current, Current is normalized to the maximum of either WT alone (I(C6)/I(Hv1)) or the WT co-expressed with a tether without toxin (I(C6)/I(notox));

FIG. 8F shows FRET measurements between Hv1-TFP and mVenus transmembrane without toxin or with C6, Normalized fluorescence versus time is fit with a single exponential decay to determine the time constants for fluorescence decay with or without toxin; and

FIG. 8G shows average taus measured with the tether without toxin and with the tether with C6 from the fit in FIG. 8F, Increase in decay rate indicates FRET and indicates an interaction between C6 and Hv1.

FIG. 9A-C: Demonstrates that Hv1 modulating agent C6 targets an S3-S4 external loop region of hHv1:

FIG. 9A illustrates sequence alignments of Ciona intestinalis Hv1 (CiHv1, yellow), human Hv1 (hHv1, cyan), a chimeric Hv1 where an S3-S4 external loop region from hHv1 replaces a corresponding region from CiHv1 (hS3S4CiHv1), a chimeric Hv1 where an S3-S4 external loop region from CiHv1 replaces an hHv1 loop region, as well as three different loop lengths transferred (ShortN, ShortNC, shortC), If C6 blocks (YES) or does not (NO) block the channel is indicated, X indicates that proton currents were not measurable;

FIG. 9B demonstrates a representative trace (left) for CiHv1 with 1 μM C6 (red trace) or without (black trace), a representative trace (middle) for hS3S4CiHv1 which is sensitive to C6 (red trace, 1 μM C6), and a representative trace (right) for CiS3S4hHv1 which is insensitive to C6 (red trace, 1 μM C6), Black traces are current without any applied peptide; and

FIG. 9C demonstrates results from a cysteine scan of part of the transferred epitope, Bars are the amount of current with 1 μM C6 normalized to current without toxin (Itox/Ictr), Many residues show decreased affinity but only G199 and E192 show dramatically different effects to the WT (first bar).

### DEFINITIONS

Component: The term “component” as used herein refers to a relevant part, portion, or moiety of an entity of interest. For example, in some embodiments, an entity of interest may be a polypeptide component.

Comprising: A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

Corresponding to: As used herein, the term “corresponding to” designates the position/identity of a structural element in a compound or composition through comparison with an appropriate reference compound or composition. For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190^{th} amino acid in a particular amino acid chain but rather corresponds to the residue found at 190 in the reference polypeptide; those of ordinary skill in the art readily appreciate how to identify “corresponding” amino acids.

Engineered: In general, the term “engineered” refers to the aspect of having been manipulated by the hand of man. For example, a polynucleotide is considered to be “engineered” when two or more sequences, that are not linked together in that order in nature, are manipulated by the hand of man to be directly linked to one another in the engineered polynucleotide. For example, in some embodiments of the present invention, an engineered polynucleotide comprises a regulatory sequence that is found in nature in operative association with a first coding sequence but not in operative association with a second coding sequence, is linked by the hand of man so that it is operatively associated with the second coding sequence. Comparably, a cell or organism is considered to be “engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). As is common practice and is understood by those in the art, progeny of an engineered polynucleotide or cell are typically still referred to as “engineered” even though the actual manipulation was performed on a prior entity.

Inhibitor Cysteine Knot (ICK)-like structural motif: As used herein, the term “inhibitor cysteine knot (ICK)-like structural motif” designates a peptide structure that has substantial structural similarity to an ICK structural motif. In some embodiments, an ICK-like structural motif has three disulfide bridges. In some embodiments, an ICK-like structural motif has two, one, or zero disulfide bridges. In some embodiments, an ICK-like structural motif has three beta strands. In some embodiments, an ICK-like structural motif has two, one, or zero beta strands. In some embodiments, an ICK-like structural motif has an amino acid sequence with six conserved cysteine residues of an ICK structural motif. In some embodiments, an ICK-like structural motif has an amino acid sequence with 5, 4, 3, 2, 1, or 0 conserved cysteine residues of an ICK structural motif.

Hv1 associated disease or condition: As used herein, the phrase “Hv1 associated disease or condition” refers to a disease, disorder, or condition in which cells exhibit relatively abnormal, uncontrolled, or undesired Hv1 channel function. Abnormal or uncontrolled Hv1 function may arise from, among other mechanisms, dysregulatd phosphorylation, differential isoform expression, or single nucleotide polymorphisms (SNPs) that alter Hv1 properties. In some embodiments, abnormal or uncontrolled Hv1 function includes abnormal activation or opening of Hv1 channels. In some embodiments, abnormal or uncontrolled Hv1 function includes abnormal closing of Hv1 channels. In some embodiments, cells that exhibit abnormal or uncontrolled Hv1 function display an abnormal level or regulation of transmembrane proton flux, transmembrane voltage and/or transmembrane pH gradient (ΔpH, defined as pHₒ-pHᵢ). In some embodiments, such cells display an abnormal level or regulation of NOX enzyme activity and/or reactive oxygen species (ROS) production. A variety of types of Hv1 associated diseases or conditions may exist, for example, inflammation, autoimmunity, cancer, asthma, brain damage in ischemic stroke, Alzheimer's disease, infertility, and numerous other conditions. In some embodiments, an Hv1 associated disease or condition refers to a condition in which Hv1 channel function is within normal, but undesired range. For example, an Hv1 associated disease or condition may refer to a condition in which changing Hv1 function would achieve a more preferred physiological outcome than not changing Hv1 function. For example, suppression of Hv1 function in human sperm may be used as a form of birth control to block fertilization.

Library: As used herein, the term “library” refers to a collection of members. A library may be comprised of any type of members. For example, in some embodiments, a library comprises a collection of phage particles. In some embodiments, a library comprises a collection of peptides. In some embodiments, a library comprises a collection of cells. A library typically includes diverse members (i.e., members of a library differ from each other by virtue of variability in an element, such as a peptide sequence, between members). For example, a library of phage particles can include phage particles that express unique peptides. A library of peptides can include peptides having diverse sequences. A library can include, for example, at least 10¹, 10², 10³, 10⁴, 10⁵, 10⁶, 10⁷, 10⁸, 10⁹, or more unique members.

Modulate: The term “modulate” is used to refer to the characteristic of changing the state and/or nature of an entity of interest. For example, a particular agent is considered to modulate an entity of interest if the presence, level, and/or form of the agent correlates with a change in the presence, level, and/or form of the entity of interest. In some embodiments, to modulate means to increase activity. In some embodiments, to modulate means to antagonize, inhibit, or reduce activity. In some embodiments, modulation involves binding or direct interaction between a modulator and the entity of interest. In some embodiments, to modulate means to affect level of a target entity of interest; alternatively or additionally, in some embodiments, to modulate means to affect activity of a target entity without affecting level of the target entity. In some embodiments, to modulate means to affect both level and activity of a target entity of interest. In some embodiments, effects of a modulator are apparent at the level of the whole-cell, tissue, system (e.g. immune system), or whole organism.

Pharmaceutical Composition: As used herein, the term “pharmaceutical composition” refers to a composition that is suitable for administration to a human or animal subject. In some embodiments, a pharmaceutical composition comprises an active agent formulated together with one or more pharmaceutically acceptable carriers. In some embodiments, the active agent is present in a unit dose amount appropriate for administration in a therapeutic regimen. In some embodiments, a therapeutic regimen comprises one or more doses administered according to a schedule that has been determined to show a statistically significant probability of achieving a desired therapeutic effect when administered to a subject or population in need thereof. In some embodiments, a pharmaceutical composition may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces. In some embodiments, a pharmaceutical composition is intended and suitable for administration to a human subject. In some embodiments, a pharmaceutical composition is sterile and substantially pyrogen-free.

Polypeptide: As used herein, the term “polypeptide” refers to any polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide is referred to as a “peptide.”

Substantial identity: As used herein, the term “substantial identity” refers to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul et al., Basic local alignment search tool, J. Mol. Biol., 215(3): 403-410, 1990; Altschul et al., Methods in Enzymology; Altschul et al., Nucleic Acids Res. 25:3389-3402, 1997; Baxevanis et al., Bioinformatics: A Practical Guide to the Analysis of Genes and Proteins, Wiley, 1998; and Misener, et al, (eds.), Bioinformatics Methods and Protocols (Methods in Molecular Biology, Vol. 132), Humana Press, 1999. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

Substantial structural similarity: As used herein, the term “substantial structural similarity” refers to presence of shared structural features such as presence and/or identity of particular amino acids at particular positions. In some embodiments the term “substantial structural similarity” refers to presence and/or identity of structural elements (for example: loops, sheets, helices, H-bond donors, H-bond acceptors, glycosylation patterns, salt bridges, and disulfide bonds). In some embodiments, the term “substantial structural similarity” refers to three dimensional arrangement and/or orientation of atoms or moieties relative to one another (for example: distance and/or angles between or among them between an agent of interest and a reference agent).

Toxin: As used herein, the term “toxin” refers to all peptides and/or proteins, of any amino acid length and sequence, in either monomeric or multimeric forms, naturally present in animal venoms or poisons and their non-venom homologues. Non-venom homologues include any molecule present outside of a venom gland or not used as a venom component but similar in sequence, structure and/or function to toxins. Animal toxins include all molecules identified or inferred by any means (e.g., physical, chemical, biochemical, genetic, genomic, proteomic) from animal venoms or poisons, including but not limited to isolation from crude venoms, isolation from venom gland tissues or extracts, identification based on venom gland proteome/proteomics, venome/venomics, transcriptome, and/or EST analysis. In some embodiments, a toxin is a toxin from a venom or poison of a centipede, lizard, scorpion, sea anemone, snail, snake, spider, or toad. In some embodiments, the amino acid sequence of a toxin can be a sequence that encodes an expressed and/or active toxin, or a sequence showing substantial identity thereto. In some embodiments, the amino acid sequence of a toxin is substantially identical to that of a wild-type toxin. In some embodiments, the amino acid sequence of a toxin is less than 100, 90, 80, 70, 60, 50, 40, 30, 20 or fewer amino acids long. In some embodiments, the amino acid sequence of a toxin is more than 5, 6, 7, 8.9, 10, 11, 12, 13, 14, 15, 16, 17, 18, 19, 20, 21, 22, 23, 24, 25, 26, 27, 28, 29, 30 or more amino acids long. Representative toxins, and their amino acid sequences and source designations are presented in Table 1.

Toxin Sequence Element: The phrase “toxin sequence element” is used herein to refer to a stretch of amino acid sequence, typically at least 5 amino acids in length, that corresponds to an element found in a wild-type toxin. In some embodiments, a toxin sequence element has a length within a range of about 5 to about 100 amino acids. In some embodiments, a toxin sequence has a length of about 5, 6, 7, 8, 9, 10, 11, 12, 13, 14, 15, 16, 17, 18, 19, 20, 21, 22, 23, 24, 25, or more amino acids. In some embodiments, a toxin sequence element has a length within a range of about 5 to about 25 amino acids. In some embodiments, a toxin sequence element differs from a corresponding sequence element found in the wild-type toxin; for example, in some embodiments, a toxin sequence element differs from its corresponding wild-type sequence element, by a sequence variation that includes an addition, substitution, or deletion of at least one amino acid residue. In some embodiments, the variation alters (e.g., adds, substitutes or deletes) 1, 2, 3, 4, 5 or more residues. In some embodiments, the variation alters exactly 1 residue. In some embodiments, the variation alters exactly 2 residues. In some embodiments, the variation alters exactly 3 residues. In some embodiments, the variation alters not more than 5, 4, 3, 2, or 1 residues. In some embodiments, the variation alters fewer than 15%, 14%, 13%, 12%, 11%, 10%, 9%, 8%, 7%, 6%, 5%, 4%, 3%, 2%, or 1% of the relevant residues. In some embodiments, a toxin sequence element corresponds to a full-length toxin. In some embodiments, a toxin sequence element corresponds to a full-length reference wild-type toxin. In some embodiments, a toxin sequence element corresponds to a portion of a reference wild-type toxin. In some embodiments, a toxin sequence element corresponds to a portion of a wild-type reference toxin, which portion is bounded on at least one end by a cysteine residue (e.g., a cysteine residue that, in the wild-type toxin, may participate in a disulfide bond).

Wild-type: As used herein, the term “wild-type” refers to a form of an entity (e.g., a polypeptide or nucleic acid) that has a structure and/or activity as found in nature in a “normal” (as contrasted with mutant, diseased, altered) state or context. In some embodiments, more than one “wild type” form of a particular polypeptide or nucleic acid may exist in nature, for example as “alleles” of a particular gene or normal variants of a particular polypeptide. In some embodiments, that form (or those forms) of a particular polypeptide or nucleic acid that is most commonly observed in a population (e.g., in a human population) is the “wild-type” form.

### DETATLED DESCRIPTION

### Hv1 Channel

The voltage-gated proton channel (Hv1), also known as the hydrogen voltage-gated channel 1 (HVCN1), is a protein that in humans is encoded by the HVCN1 gene. There are at least ten species with functionally confirmed Hv1 genes, including human (hHv1) and mouse (mHv1), in addition to several species with predicted Hv1 genes that have not yet been confirmed by expression and electrophysiology studies. Among its functions, Hv1 transports protons (H⁺) across cell membranes (DeCoursey, T. E. “The voltage-gated proton channel: a riddle, wrapped in a mystery, inside an enigma.” Biochemistry 54(21): 3250-68 (2015)). In humans, the Hv1 protein is expressed in a variety of tissues and body systems, including the immune system, the circulatory system, and the reproductive system. In these and other areas, Hv1 plays important physiological functions, such as regulation of cell charge and pH. In the present disclosure the terms Hv1 channel and Hv1 are equivalent.

Hv1 belongs to a superfamily of voltage-gated ion channels. Similar to other voltage-gated ion channels, Hv1 is a transmembrane protein that facilitates the transfer of ions (e.g. H⁺) across cell membranes. Also like other voltage-gated ion channels, Hv1 has a voltage sensor domain (VSD). However, Hv1 channels also have several unique features that distinguish them from other voltage-gated ion channels. For example, Hv1 channels are exquisitely selective for protons, whereas other ion channels such as potassium channels have some permeability to other ions besides K⁺.

According to the National Center for Biotechnology Information (NCBI) Gene database, there are three transcript variants for human HVCN1. Variant 1 (NM_001040107.1) represents the longest transcript. Variant 2 (NM_032369.3) differs in the 5′ untranslated region (UTR) compared to variant 1. Variants 1 and 2 encode the same protein (isoform 1), which is 273 amino acids. Variant 3 (NM_001256413.1) differs in the 5′ UTR, lacks a portion of the 5′ coding region, and initiates translation at a downstream start codon compared with variant 1. The resulting protein (isoform 2) is shorter (253 amino acids) and has a distinct N-terminus compared to isoform 1. The longer isoform (isoform 1) is considered to be most widely expressed, while the shorter isoform (isoform 2) has been found only in B-lymphocytes and exhibits functionally important differences compared to the full-length protein. Recently, another Hv1 isoform (Hv1Sper, post-translationally cleaved) was reported in human sperm. At least seven validated, nonsynonymous single-nucleotide polymorphisms (SNPs) for human HVCN1 have been identified. Only two of these seven have a frequencies above 1%.

### Hv1 Channel Structure

The structure of the Hv1 channel differs from other voltage-gated ion channels. Other voltage-gated ion channels consist of six transmembrane segments, with segments S1-S4 constituting the VSD that detects changes in membrane potential and S5-S6 forming the pore domain responsible for selective ion permeation. In contrast, Hv1 channels lack the pore domain (S5-S6). Instead, Hv1 channels have the first four transmembrane segments (S1-S4) and assemble as a dimer with each subunit containing its own permeation pathway. The N-terminus and C-terminus of Hv1 are on the cytoplasmic side. The main region of attachment of the Hv1 channel dimer is in the intracellular C-terminus.

Hv1 from several multicellular species (human, mouse, and the sea squirt Ciona intestinalis) exist as dimers, whereas Hv1 in several unicellular species are predicted to exist as monomers. When Hv1 from species with dimeric channels are forced to express as monomers, the channels open (activate) several-fold faster than dimeric forms and their g_{H}-V relationship is somewhat (10-15 mV) more positive. The promoters of each monomer in an Hv1 channel dimer gate cooperatively, such that both promoters must undergo voltage-induced conformational change before either conduction pathway is open. Consequently, the probability that dimeric Hv1 will open is more dependent on voltage compared to the monomeric form.

Certain amino acids may be important for Hv1 selectivity for protons. The acidic amino acid aspartate at position 112 (Asp 112), which is located in the middle of the S1 transmembrane helix, is one such amino acid important for Hv1 proton selectivity. Mutating Asp 112 to any other amino acid except Glu (another acidic amino acid) converts hHv1 channel into an anion channel. Asp 112 interacts with arginine (Arg208) in the S4 segment via two hydrogen bonds, with Asp or Arg protonated. Introducing a hydronium ion, H3O⁺, into either configuration results in protonation of Asp, breaking of the hydrogen bonds, and resulting in a neutral water molecule that mediates interactions in AspH⁰—H₂O⁰-Arg⁺. From this protonated selectivity filter configuration, reprotonation of the water molecule results in net H⁺ permeation. Therefore, the unique abilities of protons to travel with a water molecule and to transfer readily and reversibly with other groups is exploited by Hv1 in achieving proton selectivity. Proton selectivity can also be preserved when Asp is replaced with Glu, or when Arg is replaced with Lys.

Besides the selectivity filter, there is another hydrophobic region in hHv1 predicted by molecular dynamic simulations. This second region is a highly conserved Phe150. The proton may inject its own water wire through this hydrophobic region. Thus, protons are uniquely able to open the selectivity filter and to hydrate dry regions of the pore.

### Hv1 Channel Mechanism

Hv1 channels are uniquely selective for protons, with detectably no other ion permeation. The requirement of such selectivity is crucial because the concentration of protons in mammalian cells or bodily fluids is orders of magnitude lower than that of other major cations like Na⁺ and K⁺. As already discussed above, Hv1 channel selectivity is dependent on specific amino acids, including Asp 112 and Arg208.

The primary determinants of Hv1 channel activation are transmembrane voltage and transmembrane pH gradient (ΔpH, defined as pHₒ-pHᵢ). Hv1 opens at relatively positive transmembrane voltages (i.e. depolarization), but voltage-dependence is strongly modulated by pH. When the cytosol becomes more acidic relative to the extracellular or intraluminal space, the entire conductance-voltage relationship of the channel shifts by 40 mV to more negative voltages for each unit increase in ΔpH. Conversely, when the extracellular or intraluminal side becomes more acidic than the cytosol, the conductance-voltage relationship shifts by 40 mV to more positive voltages for each unitary change in ΔpH. Parameters that are useful in determining Hv1 channel activation include: (1) the membrane voltage (measured on the cytosolic side, relative to the extracellular or luminal side); (2) the cytosolic pH (whereby acidification favors activation at any given voltage); and (3) the extracellular or intra-luminal pH (whereby acidification opposes activation at any given voltage). Three charged Arg residues in the S4 transmembrane segment of Hv1 confer voltage dependency, while the structural basis for pH sensing is not fully understood.

Besides voltage and ΔpH, other parameters can also influence Hv1 channel activation. For example, phosphorylation of the channel by PKC can produce an enhanced responsiveness mode, allowing for more channels to open more quickly. PKC phosphorylates Hv1 at Thr29 located in the intracellular N-terminus. A situational example of enhanced gating of Hv1 is phagocyte exposure to pathogenic stimuli, such as bacteria. A diversity of stimuli can induce enhanced gating, including chemotactic peptides such as fMLF in neutrophils, lipopolysaccharide (LPS) in dendritic cells, IgE in basophils, IL-5 in eosinophils, and arachidonic acid in neutrophils and eosinophils. Such enhanced Hv1 gating is only functional in certain cells. The intensity of enhanced gating response may be associated with the presence of an active NADPH oxidase complex. Additionally, slower tail current decay (channel closing), is temporally correlated with NADPH oxidase activity. Enhanced gating makes Hv1 channels more likely to open or remain open, thereby requiring a smaller stimulus to activate H⁺ flux. Hv1 proton currents are also sensitive to temperature, and have a small (15 fF) unitary conductance.

### Hv1 Channel Expression

Hv1 channels have been identified directly by voltage-clamp recordings in many primary tissue cell types, including neutrophils, basophils, eosinophils, cardiac fibroblasts, cultured myotubes, tracheal epithelium, and monocytes. Neutrophil and eosinophil granulocytes express the highest levels of Hv1.

In most cells, Hv1 is expressed in plasma membranes, though there is evidence that Hv1 can also be expressed on intracellular membranes such as Golgi membranes in some cells. Full-length Hv1 can be detected in human granulocytes by western blot as a 30 kDa monomer or 70 kDa dimer. Based on immunocytochemistry, Hv1 partially colocalizes with NOX2 in the membrane of intracellular granules and in the plasma membrane.

### Hv1 Channel Functions

Functions of Hv1 channels differ depending on the cells in which they are expressed. Cells in which high activity and a physiological function for Hv1 channels have been documented include immune cells, central nervous system cells, airway epithelia, spermatozoa, and cardiac fibroblasts. Under normal circumstances, when Hv1 channels open, H⁺ efflux occurs and thereby increases pHᵢ, decreases pHₒ and hyperpolarizes the membrane potential. These changes can have different consequences in different cells.

In some cells, Hv1 channel expression and function is closely linked to expression and function of the enzyme NADPH oxidase (NOX), of which there are four isoforms: NOX1, NOX2, NOX3 and NOX4. NOX is a membrane-bound enzyme that transfers electrons from NADPH across cell membranes and couples these electrons to molecular oxygen to produce superoxide anion. In some locations, superoxide can undergo further reactions to generate reactive oxygen species (ROS). One of the functions of Hv1 linked to NOX activity is the extrusion of protons to compensate for the loss of electrons, as discussed in more detail below.

Hv1 expression and/or function has been detected in both innate and adaptive immune cells. A major role of Hv1 channels is in the phagosome, an intracellular organelle in white blood cells where pathogens such as bacteria are engulfed and destroyed. The primary role for Hv1 channels in the phagosome is to allow NOX2 (the NADPH oxidase enzyme complex) to produce large quantities of reactive oxygen species (ROS) to kill pathogens, in a process called “respiratory burst”. During the respiratory burst, NOX enzymes catalyze the transfer of electrons from NADPH across the plasma membrane to reduce molecular oxygen to O²⁻, generating two protons in the cytoplasm. The resulting depolarization and cytoplasmic acidification inhibits NOX2 activity. Depolarization opens Hv1 channels to sustain NOX2 activity by extruding protons from the cytoplasm, thereby maintaining physiological membrane potential and re-establishing normal pH. Such contributions of Hv1 channels to NOX2-dependent ROS release are characterized in granulocytes and in particular neutrophils. H⁺ current has also been detected in basophils. In these cells, IgE stimulates Hv1 channels, which facilitate release of histamine. Hv1 may also participate in ROS production and/or histamine release by mast cells.

B lymphocytes of the adaptive immune system, which are responsible for antibody production, express Hv1 protein. It has been suggested that Hv1 mediates signaling in the antibody maturation process upon B-cell receptor activation by antigen binding. For example, Hv1 channels may be required for ROS production by NOX2 in mature B lymphocytes upon antigen stimulation. Given the involvement of Hv1 in B cell receptor signaling, inhibitors of Hv1 may be useful for treating autoimmune diseases and B cell malignancies.

Hv1 channels are also expressed in T lymphocytes, which are cells of the adaptive immune system that recognize antigens presented by major histocompatibility complex I or II. Here, Hv1 may function to facilitate ROS production and help regulate the number of activated T lymphocytes, thereby opposing an autoimmune phenotype.

Expression and activity of Hv1 has been confirmed in human sperm. Functional data indicate that Hv1 activity may be necessary for sperm activation and mobility of human sperm to achieve fertilization. The process that prepares sperm to fertilize an oocyte is called capacitation, a kind of maturation process that is triggered by an increase in intracellular pH and ROS. Changes related to capacitation include: changes in sperm motility, decrease of cholesterol in the membranes, increase of tyrosine phosphorylation in several proteins, and maturation of the sperm response to progesterone. Interestingly, seminal fluid has an unusually high concentration of Zn²⁺ (which is known to inhibit Hv1), whereas the female reproductive tract has low Zn²⁺ concentrations. It is hypothesized that on arrival of sperm in the female reproductive tract, Hv1 becomes activated and cooperates with the sperm-specific Ca²⁺ channel CatSper and NOX5 to activate sperm effector functions such as sperm movement, capacitation, sperm-zona pellucida interaction, acrosome reaction, and sperm-oocyte fusion.

There is evidence to suggest the presence of Hv1 channels in mammalian brain tissue. Without wishing to be bound by any particular theory, the present disclosure proposes that Hv1 channels may play a neuroprotective role by extruding protons from metabolically active neurons and regulating neuronal pH homeostasis. Hv1 expression has been detected in human microglia, the macrophage-like cells of the central nervous system. Hv1 in microglia may contribute to CNS disease by supporting NOX function. Microglia can become activated in acute and chronic brain disorders including brain injury, ischemia, and neurodegeneration. The expression and function of Hv1 is correlated with the expression and function of NOX2. Hv1 channels may support the activity of NOX2 in microglia by extruding excess protons from the cytoplasm. Oxidative stress, at least in part due to ROS generation by NOX, can contribute to the development of CNS disease. Hv1 inhibition could be beneficial for the treatment of neurodegenerative processes accompanied by excessive production of ROS by microglia, such as stroke, Alzheimer's disease, Parkinson's disease, and amyotrophic lateral sclerosis, among others.

The present disclosure proposes that NOX-independent Hv1 channel functions may also exist, given that certain cell types (e.g., basophil granulocytes) that exhibit Hv1 channel activity are not known to express NOX.

Evidence indicates that Hv1 also functions in airway epithelia (e.g. tracheal epithelia) in functions such as providing a protective mechanism through acidification of the airway surface liquid. Hv1 may also function in cardiac fibroblasts, though its function in these cells is not fully known. Some experimental data also suggests Hv1 function in monocytes and macrophages. Hv1 channels may promote osteoclast cell function, for instance by promoting bone resorption by osteoclast cells. Hv1 channels may also mediate antigen presentation by dendritic cells.

Many other tissues and cells not mentioned here also express Hv1 channels, some at relatively low levels. For most of the cell types and tissues that have been reported to express low levels of Hv1 channels, a specific function has not been assigned.

Given the widespread expression and function of Hv1 channels, it may not be surprising that channel dysfunction can cause or enhance pathologic states. Moreover, genomic studies have identified the HVCN1 gene as being relevant to multiple diseases. For example, HVCN1 has been associated with Crohn's disease activity and cystic fibrosis. A study in HVCN1 knockout rats indicated that Hv1 may contribute to the development of hypertension and renal disease with a high-salt diet. The link between Hv1 function and ROS production provides some insight on the mechanism of some Hv1-associated disorders. Excessive ROS production is thought to cause local tissue damage and contribute to several pathological conditions, including atherosclerosis, ischemic stroke, Parkinson's disease, ischemic liver disease, Alzheimer's disease, and aging. A study on ischemic stroke has confirmed that Hv1 can exacerbate brain damage by facilitating production of ROS by NOX in microglia. Moreover, it was recently shown that Hv1-deficient mice are protected in models of stroke, suggesting that pharmacological inhibition of Hv1 channels may have neuroprotective benefits (see Seredenina, T., et al. “Voltage-gated proton channels as novel drug targets: from NADPH oxidase regulation to sperm biology.” Antioxid Redox Signal. 10; 23(5): 490-513 (2015) and references cited therein).

Hv1 channels may contribute to the malignancy of several cancers, including breast cancer, colorectal cancer and leukemia. For example, higher levels of Hv1 expression occur in breast cancer cell lines with greater metastatic likelihood and knockdown of Hv1 in breast cancer cell lines reduced proliferation and invasiveness. In human patients, a high level of Hv1 expression was correlated with poor prognosis. One mechanism by which Hv1 contributes to cancer cell malignancy is related to the abnormal metabolism of cancer cells, which use glycolysis in preference to oxidative phosphorylation even in the presence of adequate oxygen. This altered metabolism creates a buildup of lactic acid that acidifies the cells, thus requiring enhanced activity of H⁺ extrusion to prevent cell death.

The particular isoform of Hv1 that is expressed may contribute to certain malignancies. The levels of the short isoform of Hv1 are higher in malignant B cell lines as compared to normal B lymphocytes. Moreover, the short isoform comprises approximately one-third of the Hv1 protein in malignant B cells from patients with chronic lymphocytic leukemia. The enhanced gating response is substantially more pronounced in the short compared to the long isoform of Hv1. Hv1 channel activity, proliferation and cell migration are all promoted by the expression of the short isoform.

### Hv1 Channel Modulators

Modulation of Hv1 channel activity is an attractive strategy for treating Hv1-related pathologies, including the ones described above. For example, agents that modulate Hv1 may be expected to ameliorate inflammation, allergies, autoimmunity, cancer, asthma, brain damage from ischemic stroke, Alzheimer's disease, infertility, and numerous other conditions. Depending on the disease and affected tissue, either Hv1 activation or inhibition could prove useful. However, clinically compatible Hv1 modulators are not known. Therefore, there is an unmet need in identifying potent and selective modulators of Hv1.

One of the challenges in creating modulators of Hv1 channels stems from the channel structure. The extracellular loops of hHv1 are fewer than a dozen amino acids, resulting in a relatively small extracellular portion of the Hv1 molecule to which drugs can bind. For example, this limits the epitope possibilities for antibodies to bind externally. Additionally, inhibition of Hv1 by physical occlusion is also a challenge, since the channel is structured to be closely packed and prevent other ions from permeating.

In some instances, such as autoimmune disease and male infertility, Hv1 activation may be an attractive pharmacological strategy. Unsaturated long-chain fatty acids such as oleic acid and arachidonic acid have been shown to enhance Hv1 proton currents. This appears to be a direct pharmacological activation of Hv1. Arachidonic acid has been observed to activate a proton conductance in phagocytes. However, arachidonic acid can also activate multiple signaling pathways, which in certain cases can lead to activation of NOX enzymes and therefore indirectly activate H⁺ currents. Activators of enhanced gating can also enhance Hv1 function. In general, ion channel activators are more difficult to identify than inhibitors since binding to the channel usually produces inhibition.

Zn²⁺ and other polyvalent cations are known to inhibit Hv1 channels. Hv1 channels can be blocked by Zn²⁺ at concentrations ranging from 100 nM to 1 mM depending on the extracellular pH and on the presence of other polyvalent cations. The mechanism by which Zn²⁺ inhibits Hv1 involves Zn²⁺ competing with H⁺ for binding to the external surface of Hv1 channels. Two His residues (His140 and His193) located at the interface between the channel monomers coordinate Zn²⁺ in the closed channel and thereby oppose channel opening. This mechanism changes the membrane potential perceived by the channel, and therefore requires stronger voltage to elicit proton current. Zn²⁺ shifts the current-voltage relationship positively and slows the kinetics of Hv1 channel activation. However, Zn²⁺ ions are implicated in many other physiological processes, and therefore the usefulness of Zn²⁺ as a specific H⁺ channel blocker is limited.

There are no documented high-affinity blockers of Hv1 channels that originate in venom or toxin. Tarantula toxins, including hanatoxin, can inhibit Hv1 at low micromolar concentrations by interacting with the S3 and S4 helices from the membrane interior and shifting the g_{H}-V relationship in the positive direction. However, hanatoxin is not specific for Hv1. Different voltage-sensing proteins, including Hv1 and other ion channels, contain the highly conserved voltage sensor regions composed of S3 and S4 helices, termed the paddle motif. Binding of hanatoxin to the paddle motif inhibits ion fluxes through various voltage-dependent ion channels besides Hv1.

Guanidine derivatives have been shown to inhibit depolarization-induced H⁺ current (see Seredenina, T., et al. “Voltage-gated proton channels as novel drug targets: from NADPH oxidase regulation to sperm biology.” Antioxid Redox Signal. 10; 23(5): 490-513 (2015); DeCoursey, T. E. “The voltage-gated proton channel: a riddle, wrapped in a mystery, inside an enigma.” Biochemistry 54(21): 3250-68 (2015); and references cited therein). The derivative 2GBI [2-guanidinobenzimidazole (IC₅₀=38 μM)] was found to have an intracellular site of action and to only bind when the channel was open. Other identified derivatives include 1-(1,3-benzothiasol-2-yl)guanidine and 5-chloro-2-guanidinobenzimidazole. Guanidine derivatives have shown neuroprotective potential in an in vitro model of ischemia. The biggest challenge for pharmaceutical application of guanidine derivatives is their intracellular site of action.

Several other compounds have been observed to block H⁺ currents. Examples of such other compounds include weak bases (e.g. 4-aminopyridine, amiloride, verapamil or D600), tricyclic antidepressants (imipramine, amitryptiline, and desipramine), the selective serotonin reuptake inhibitor fluoxetine, the morphine-derivative dextromethorphan (DM), and a tea catechin flavonoid EGCG. These other potential inhibitors have several drawbacks, including mechanisms of action that do not directly involve Hv1 channels, multiple other targets, and effective concentrations that are too high to be of pharmaceutical interest.

To-date, there are no selective inhibitors of Hv1 channels. There is an unmet need to develop such inhibitors, especially ones that are compatible with clinical use.

### Hv1 Modulating Agents

### Hv1 Modulating Agent Activities

The present disclosure provides agents that modulate Hv1. Among other things, the present disclosure provides agents that, for example, modulate one or more Hv1 activities when contacted with an Hv1 channel, for example, in vitro and/or in vivo. In some embodiments, Hv1 modulating agents modulate Hv1 activities of Hv1 monomers and dimers with similar IC₅₀. In some embodiments, Hv1 modulating agents specifically bind Hv1. In some embodiments, Hv1 modulating agents inhibit Hv1. In some embodiments, Hv1 modulating agents activate Hv1.

In some embodiments, Hv1 modulating agents do not physically occlude Hv1 channels. In some embodiments, Hv1 modulating agents bind to Hv1 but do not bind to other voltage-gated channels or other ion channels.

In some embodiments, Hv1 modulating agents bind to the external surface of Hv1. In some embodiments, Hv1 modulating agents target or bind to the S3-S4 external loop region of hHv1. For example, Hv1 modulating agents may bind to hHv1 at amino acid residues I183 to L204. In some embodiments, Hv1 modulating agents bind to regions of hHv1 comprising an amino acid sequence corresponding to ILDIVLLFQEHQFEALGLLILL (SEQ ID NO: 111).

In some embodiments, Hv1 modulating agent binding to Hv1 is reversible. In some embodiments, Hv1 modulating agent binding to Hv1 may be irreversible. In some embodiments, Hv1 modulating agent binding to Hv1 is strong but not irreversible.

In some embodiments Hv1 modulating agents bind to open Hv1 channels. In some embodiments, Hv1 modulating agents bind to closed channels. In some embodiments, affinity of Hv1 modulating agents for closed states of Hv1 is about 1 nM. In some embodiments, affinity of Hv1 modulating agents is lower for open states of the channel (e.g. about 200 nM) as compared to closed states of the channel. In some embodiments, Hv1 modulating agents slow opening of closed states of Hv1 even as they unbind.

In some embodiments, provided Hv1 modulating agents may change transmembrane voltage of a cell. Hv1 modulating agents may hyperpolarize the membrane potential. Hv1 modulating agents may depolarize the membrane potential. Effects of Hv1 modulating agents may be measured by, for example, direct electrophysiological recordings of voltage-gated proton currents, such as patch-clamp recordings.

In some embodiments, provided Hv1 modulating agents are characterized in that, for example, they decrease or block proton current. In some embodiments, Hv1 modulating agents may reduce the number or likelihood of Hv1 channel opening. In some embodiments, Hv1 modulating agents may speed up the rate of Hv1 channel closing. In some embodiments, Hv1 modulating agents may cause an Hv1 channel to require stronger voltage to elicit proton current.

In some embodiments, provided Hv1 modulating agents are characterized in that, for example, they increase proton currents and/or slow the closing of Hv1 channels. In some embodiments, Hv1 modulating agents may provide an enhanced responsiveness mode, allowing more channels to open more quickly, increasing likelihood that Hv1 channels will open or remain open, and/or reducing the stimulus required to activate H⁺ flux.

In some embodiments, Hv1 modulating agents may increase or decrease proton (H⁺) current but do not directly alter current of other ions (e.g. Na⁺, K²⁺, Ca²⁺).

In some embodiments, Hv1 modulating agents may change the transmembrane pH gradient (ΔpH, defined as pHₒ-pHᵢ). In some embodiments, provided Hv1 modulating agents may increase or decrease intracellular or cytosolic pH (pHᵢ). Thus, Hv1 modulating agents may decrease or increase the cytoplasmic acidity. In some embodiments, provided Hv1 modulating agents may increase or decrease the extracellular, intraluminal, or organelle pH (pHₒ). Thus, Hv1 modulating agents may decrease or increase the extracellular, intraluminal, or organelle acidity.

In some embodiments, Hv1 modulating agents may increase or decrease cellular ROS production.

In some embodiments, Hv1 modulating agents may increase or decrease the function and/or activity of NOX enzymes, including NOX1, NOX2, NOX3, and/or NOX4. In some such embodiments, Hv1 modulating agents may enhance or reduce the ability of NOX enzymes to transfer electrons. In some such embodiments, Hv1 modulating agents may increase or decrease the production of superoxide anion by a cell. In some such embodiments, Hv1 modulating agents may increase or decrease the quantity of ROS production mediated by NOX enzymes. In some embodiments, Hv1 modulating agents may sustain NOX activity by extruding protons from the cytoplasm.

In some embodiments, Hv1 modulating agents may alter signaling pathways that can be affected by Hv1 activity. In some embodiments, Hv1 modulating agents may affect cellular, physiological, or pathological processes that can be affected by Hv1 activity. In some such embodiments, Hv1 modulating agents may influence inflammation, allergies, autoimmunity, cancer, asthma, brain damage from ischemic stroke, Alzheimer's disease, and fertility.

In some embodiments, Hv1 modulating agents may alter sperm activity or fertilization. Hv1 modulating agents may affect sperm mobility, capacitation, sperm-zona pellucida interaction, acrosome reaction, and sperm-oocyte fusion. In a particular example, Hv1 modulating agents inhibit properties associated with sperm capacitation.

In another example, Hv1 modulating agents may alter the ability of white blood cells to fight infections. Hv1 modulating agents may alter the activity of phagosomes, NOX enzymes, or ROS production. In a particular example, Hv1 modulating agents may decrease ROS production in white blood cells.

### Hv1 Modulating Agent Structure

In some embodiments, an Hv1 modulating agent is or comprises a polypeptide. In some embodiments, a polypeptide component of an Hv1 modulating agent is 10-100 amino acids in length.

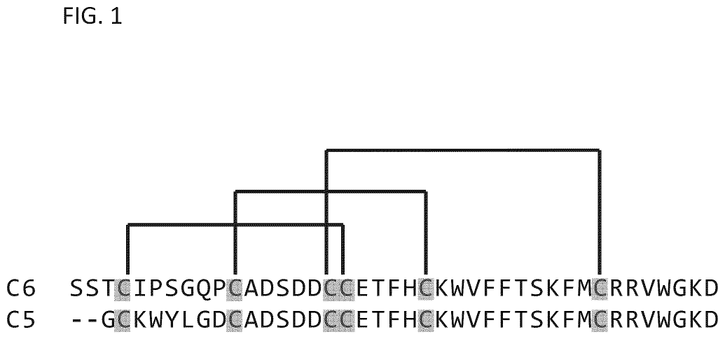

In many embodiments in which a provided Hv1 modulating agent includes a polypeptide component, the polypeptide component of the agent has an amino acid tertiary structure that is characterized as an inhibitor cysteine knot (ICK)-like structural motif. In some embodiments, a polypeptide component has a structure that has substantial structural similarity to an ICK structural motif. In some embodiments, a polypeptide component has three disulfide bridges. In some embodiments, a polypeptide component has three beta strands. In some embodiments, a polypeptide component has an amino acid sequence with six conserved cysteine residues of an ICK motif (FIG. 1).

In many embodiments in which a provided Hv1 modulating agent includes a polypeptide component, the polypeptide component of the agent has an amino acid sequence that includes one or more elements that is substantially identical to, but different from, that of wild-type toxin sequences (e.g., of a wild-type voltage sensor toxin). In some embodiments, such a sequence element has a length of about 5 to about 20 amino acids. In some embodiments, such a sequence element shows at least 40%, 50%, 60%, 70%, 80%, 90%, 91%, 92%, 93%, 94%, 95%, 96%, 97%, 98%, 99%, or 100% sequence identity with a corresponding element of a wild-type toxin. In some embodiments, a polypeptide component of a provided Hv1 modulating agent may show significant (e.g., at least 40%, 50%, 60%, 70%, 80%, 90%, 91%, 92%, 93%, 94%, 95%, 96%, 97%, or higher) overall sequence identity with, while differing from, a wild-type toxin. In some embodiments, a polypeptide component has an amino acid sequence that includes a plurality of toxin sequence elements, each of which is substantially identical to a sequence element that is found in the same, or a different, reference wild-type toxin. In some such embodiments, the plurality of toxin sequence elements are assembled in linear order so that the amino acid sequence shows overall correspondence with (e.g., shares one or more certain structural features, such as number and/or [relative] location of one or more cysteine residues) a full-length toxin. In some embodiments, a polypeptide component has an amino acid sequence that includes one or more residues found in a wild-type toxin that participates in binding by that wild-type toxin to a voltage-sensing protein.

Exemplary wild-type toxin sequences are presented in Table 1. In some embodiments, a wild-type toxin is a venom toxin. In some embodiments, a venom toxin is a toxin found in venom of organisms such as scorpion (e.g., Pandinus imperator), sea anemone, snails (e.g. Conus marmoreus), snakes, and spiders (e.g., Grammostola rosea).

TABLE 1

| SEQ | | | NCBI | |
|---|---|---|---|---|
| ID | Toxin | | Accession | |
| NO: | Name | Sequence | NO: | Animal Species |
| 1 | HwTx-IV | ECLEIFKACNPSNDQCCKSSKLVCSRKTRWCKYQI | P83303.2 | Haplopelma |
| | | | | schmidti (Chinese |
| | | | | bird spider) |
| 2 | HnTx-IV | ECLGFGKGCNPSNDQCCKSSNLVCSRKHRWCKYEI | D2Y232.1 | Haplopelma |
| | | | | hainanum (Chinese |
| | | | | bird spider) |
| 3 | HnTx-V | ECLGFGKGCNPSNDQCCKSANLVCSRKHRWCKYEI | P60975.1 | Haplopelma |
| | | | | hainanum (Chinese |
| | | | | bird spider) |
| 4 | PaurTx3 | DCLGFLWKCNPSNDKCCRPNLVCSRKDKWCKYQI | P84510.1 | Paraphysa scrofa |
| | | | | (Chilean copper |
| | | | | tarantula) |
| 5 | CcoTx1 | DCLGWFKSCDPKNDKCCKNYTCSRRDRWCKYDL | P84507.1 | Ceratogyrus |
| | | | | marshalli |
| | | | | (Straighthorned |
| | | | | baboon tarantula) |
| 6 | CcoTx2 | DCLGWFKSCDPKNDKCCKNYTCSRRDRWCKYYL | P84508.1 | Ceratogyrus |
| | | | | marshalli |
| | | | | (Straighthorned |
| | | | | baboon tarantula) |
| 7 | VSTX3 | DCLGWFKGCDPDNDKCCEGYKCNRRDKWCKYKLW | P0C2P5.1 | Grammostola rosea |
| | | | | (Chilean rose |
| | | | | tarantula) |
| 8 | TlTx1 | AACLGMFESCDPNNDKCCPNRECNRKHKWCKYKLW | P83745.1 | Theraphosa blondi |
| | | | | (Goliath |
| | | | | birdeating spider) |
| 9 | JZTX-25 | DDCLGMFSSCNPDNDKCCEGRKCDRRDQWCKWNPW | B1P1F1.1 | Chilobrachys |
| | | | | guangxiensis |
| | | | | (Chinese earth |
| | | | | tiger tarantula) |
| 10 | JZTX-27 | DCLGLFWICNYMDDKCCPGYKCERSSPWCKIDI | B1P1H2.1 | Chilobrachys |
| | | | | guangxiensis |
| | | | | (Chinese earth |
| | | | | tiger tarantula) |
| 11 | TlTx3 | DDCLGMFSSCDPNNDKCCPNRVCRVRDQWCKYKLW | P83747.1 | Theraphosa blondi |
| | | | | (Goliath |
| | | | | birdeating spider) |
| 12 | TlTx2 | DDCLGMFSSCDPKNDKCCPNRVCRSRDQWCKYKLW | P83746.1 | Theraphosa blondi |
| | | | | (Goliath |
| | | | | birdeating spider) |
| 13 | HwTx-I | ACKGVFDACTPGKNECCPNRVCSDKHKWCKWKL | P56676.2 | Haplopelma |
| | | | | schmidti (Chinese |
| | | | | bird spider) |
| 14 | GsMTx4 | GCLEFWWKCNPNDDKCCRPKLKCSKLFKLCNFSFGK | Q7YT39.1 | Grammostola rosea |
| | | | | (Chilean rose |
| | | | | tarantula) |
| 15 | Omega- | ADCGWLFHSCESNADCCENWACATTGRFRYLCKYQI | P81595.1 | Hadronyche versuta |
| | AcTx- | | | (Blue mountains |
| | Hv1b | | | funnel-web spider) |
| 16 | IpTxa | GDCLPHLKRCKADNDCCGKKCKRRGTNAEKRCR | P59868.1 | Pandinus imperator |
| | | | | (Emperor scorpion) |
| 17 | VSTX1 | ECGKFMWKCKNSNDCCKDLVCSSRWKWCVLASPF | P60980.2 | Grammostola rosea |
| | | | | (Chilean rose |
| | | | | tarantula) |
| 18 | HnTx-I | ECKGFGKSCVPGKNECCSGYACNSRDKWCKVLL | D2Y1X6.1 | Haplopelma |
| | | | | hainanum (Chinese |
| | | | | bird spider) |
| 19 | Maurocalcine | GDCLPHLKLCKENKDCCSKKCKRRGTNIEKRCR | P60254.1 | Scorpio maurus |
| | | | | palmatus (Chactoid |
| | | | | scorpion) |
| 20 | HpTX3 | ECGTLFSGCSTHADCCEGFICKLWCRYERTW | P58427.1 | Heteropoda |
| | | | | venatoria (Brown |
| | | | | huntsman spider) |
| 21 | HNTX- | ECRYWLGTCSKTGDCCSHLSCSPKHGWCVWDWTFRK | D2Y2C3.1 | Haplopelma |
| | VII | | | hainanum (Chinese |
| | | | | bird spider) |
| 22 | JZTX | GCQKFFWTCHPGQPPCCSGLACTWPTEICIDG | P0CH50.1 | Chilobrachys |
| | F4- | | | guangxiensis |
| | 32.60 | | | (Chinese earth |
| | | | | tiger tarantula) |
| 23 | HnTx- | GCKGFGDSCTPGKNECCPNYACSSKHKWCKVYL | D2Y1X9.1 | Haplopelma |
| | III | | | hainanum (Chinese |
| | | | | bird spider) |
| 24 | Toxin_KJ1 | DDCGTLFSGCDTSKDCCEGYVCHLWCKYK | P61791.1 | Heteropoda |
| | | | | venatoria (Brown |
| | | | | huntsman spider) |
| 25 | ScTx1 | DCTRMFGACRRDSDCCPHLGCKPTSKYCAWDGTI | P60991.1 | Stromatopelma |
| | | | | calceatum |
| | | | | (Featherleg baboon |
| | | | | tarantula) |
| 26 | JZTX-50 | RCIEEGKWCPKKAPCCGRLECKGPSPKQKKCTRP | B1P1B0.1 | Chilobrachys |
| | | | | guangxiensis |
| | | | | (Chinese earth |
| | | | | tiger tarantula) |
| 27 | ProTx-1 | ECRYWLGGCSAGQTCCKHLVCSRRHGWCVWDGTFS | P83480.1 | Thrixopelma |
| | | | | pruriens (Peruvian |
| | | | | green velvet |
| | | | | tarantula) |
| 28 | HmTx1 | ECRYLFGGCSSTSDCCKHLSCRSDWKYCAWDGTFS | P60992.1 | Heteroscodra |
| | | | | maculata (Togo |
| | | | | starburst |
| | | | | tarantula) |
| 29 | GxTx1E | EGECGGFWWKCGSGKPACCPKYVCSPKWGLCNFPMP | P84835.1 | Chilobrachys |
| | | | | guangxiensis |
| | | | | (Chinese earth |
| | | | | tiger tarantula) |
| 30 | GxTX-1D | DGECGGFWWKCGSGKPACCPKYVCSPKWGLCNFPMP | P84836.1 | Chilobrachys |
| | | | | guangxiensis |
| | | | | (Chinese earth |
| | | | | tiger tarantula) |
| 31 | Omega- | DDDCGWIMDDCTSDSDCCPNWVCSKTGFVKNICKYEM | P56207.1 | Hadronyche versuta |
| | AcTx- | | | (Blue mountains |
| | Hv1a | | | funnel-web spider) |
| 32 | JZTX | LCSREGEFCYKLRKCCAGFYCKAFVLHCYRN | P0CH55.1 | Chilobrachys |
| | F7- | | | guangxiensis |
| | 15.33 | | | (Chinese earth |
| | | | | tiger tarantula) |
| 33 | Tx2-9 | SFCIPFKPCKSDENCCKKFKCKTTGIVKLCRW | AAB32862.1 | Brachypelma |
| | | | | smithii (Mexican |
| | | | | red knee |
| | | | | tarantula) |
| 34 | GxTX-2 | ECRKMFGGCSVDSDCCAHLGCKPTLKYCAWDGT | P84837.1 | Chilobrachys |
| | | | | guangxiensis |
| | | | | (Chinese earth |
| | | | | tiger tarantula) |
| 35 | HpTX1 | DCGTIWHYCGTDQSECCEGWKCSRQLCKYVIDW | P58425.1 | Heteropoda |
| | | | | venatoria (Brown |
| | | | | huntsman spider) |
| 36 | SHLP-I | GCLGDKCDYNNGCCSGYVCSRTWKWCVLAGPWRR | Q86C51.1 | Haplopelma |
| | | | | schmidti (Chinese |
| | | | | bird spider) |
| 37 | JZTX- | GCGGLMAGCDGKSTFCCSGYNCSPTWKWCVYARP | P0C2X7.2 | Chilobrachys |
| | VII | | | guangxiensis |
| | | | | (Chinese earth |
| | | | | tiger tarantula) |
| 38 | JZTX-29 | ECRKMFGGCSVHSDCCAHLGCKPTLKYCAWDGTF | B1P1E4.1 | Chilobrachys |
| | | | | guangxiensis |
| | | | | (Chinese earth |
| | | | | tiger tarantula) |
| 39 | JZTX- | GCGGLMDGCDGKSTFCCSGFNCSPTWKWCVYARP | B1P1C4.1 | Chilobrachys |
| | 12.1 | | | guangxiensis |
| | | | | (Chinese earth |
| | | | | tiger tarantula) |
| 40 | Toxin_AU2 | DDCGGLFSGCDSNADCCEGYVCRLWCKYKL | P61792.1 | Heteropoda |
| | | | | venatoria (Brown |
| | | | | huntsman spider) |
| 41 | HaTx1 | ECRYLFGGCKTTSDCCKHLGCKFRDKYCAWDFTFS | P56852.1 | Grammostola rosea |
| | | | | (Chilean rose |
| | | | | tarantula) |
| 42 | HaTx2 | ECRYLFGGCKTTADCCKHLGCKFRDKYCAWDFTFS | P56853.1 | Grammostola rosea |
| | | | | (Chilean rose |
| | | | | tarantula) |
| 43 | VaTx1 | SECRWFMGGCDSTLDCCKHLSCKMGLYYCAWDGTF | P0C244.1 | Psalmopoeus |
| | | | | cambridgei |
| | | | | (Trinidad chevron |
| | | | | tarantula) |
| 44 | JzTx-XI | ECRKMFGGCSVDSDCCAHLGCKPTLKYCAWDGTFGK | P0C247.2 | Chilobrachys |
| | | | | guangxiensis |
| | | | | (Chinese earth |
| | | | | tiger tarantula) |
| 45 | HmTx2 | ECRYFWGECNDEMVCCEHLVCKEKWPITYKICVWDRT | P60993.1 | Heteroscodra |
| | | F | | maculata (Togo |
| | | | | starburst |
| | | | | tarantula) |
| 46 | JzTx- | DGECGGFWWKCGRGKPPCCKGYACSKTWGWCAVEAP | P62520.1 | Chilobrachys |
| | III | | | guangxiensis |
| | | | | (Chinese earth |
| | | | | tiger tarantula) |
| | | | | (Chilobrachys |
| | | | | jingzhao) |
| 47 | PcTx1 | EDCIPKWKGCVNRHGDCCEGLECWKRRRSFEVCVPKT | P60514.1 | Psalmopoeus |
| | | PKT | | cambridgei |
| | | | | (Trinidad chevron |
| | | | | tarantula) |
| 48 | Agelenin | GGCLPHNRFCNALSGPRCCSGLKCKELSIWDSRCL | P31328.1 | Allagelena |
| | | | | opulenta (Funnel |
| | | | | weaving spider) |
| 49 | JZTX-13 | QCGEFMWKCGAGKPTCCSGYDCSPTWKWCVLKSPGRR | B1P1C9.1 | Chilobrachys |
| | | | | guangxiensis |
| | | | | (Chinese earth |
| | | | | tiger tarantula) |
| 50 | JZTX-15 | TCYDIGELCSSDKPCCSGYYCSPRWGWCIYSTRGGR | B1P1D4.1 | Chilobrachys |
| | | | | guangxiensis |
| | | | | (Chinese earth |
| | | | | tiger tarantula) |
| 51 | Omega- | SAVCIPSGQPCPYSKYCCSGSCTYKTNENGNSVQRCD | P81599.1 | Hadronyche versuta |
| | AcTx- | | | (Blue mountains |
| | Hv1f | | | funnel-web spider) |
| 52 | HNTX- | CAAEGIPCDPNPVKDLPCCSGLACLKPTLHGIWYKHH | D2Y299.1 | Haplopelma |
| | XIX | YCYTQ | | hainanum (Chinese |
| | | | | bird spider) |
| 53 | lamda- | GCNRKNKKCNSDADCCRYGERCISTKVNYYCRPDRGP | P86399.2 | Mesobuthus eupeus |
| | MeuTx | | | (Lesser Asian |
| | | | | scorpion) |
| 54 | JZTX-24 | VCRGYGLPCTPEKNDCCQRLYCSQHRLCSVKA | B1P1F0.1 | Chilobrachys |
| | | | | guangxiensis |
| | | | | (Chinese earth |
| | | | | tiger tarantula) |
| 55 | HwTx-X | KCLPPGKPCYGATQKIPCCGVCSHNKCT | P68424.2 | Haplopelma |
| | | | | schmidti (Chinese |
| | | | | bird spider) |
| 56 | JZTX-21 | CGGWMAKCADSDDCCETFHCTRFNVCGK | B1P1E6.1 | Chilobrachys |
| | | | | guangxiensis |
| | | | | (Chinese earth |
| | | | | tiger tarantula) |
| 57 | magi-11 | SCKLTFWRCKKDKECCGWNICTGLCIPP | Q75WH2.1 | Macrothele gigas |
| | | | | (Spider) |
| 58 | SGTx1 | TCRYLFGGCKTTADCCKHLACRSDGKYCAWDGTF | P56855.1 | Stromatopelma |
| | | | | calceatum |
| | | | | griseipes (Feather |
| | | | | leg baboon |
| | | | | tarantula) |
| 59 | JZTX-44 | ECKWYLGDCKAHEDCCEHLRCHSRWDWCIWDGTF | B1P1G8.1 | Chilobrachys |
| | | | | guangxiensis |
| | | | | (Chinese earth |
| | | | | tiger tarantula) |
| 60 | HNTX-VI | ECKYLWGTCEKDEHCCEHLGCNKKHGWCGWDGTF | P0CH70 | Haplopelma |
| | | | | hainanum (Chinese |
| | | | | bird spider) |
| 61 | GsAF_I | YCQKWLWTCDSERKCCEDMVCRLWCKKRL | P61408.1 | Grammostola rosea |
| | | | | (Chilean rose |
| | | | | tarantula) |
| 62 | GrTx1 | YCQKWMWTCDSKRKCCEDMVCQLWCKKRL | P85117.1 | Grammostola rosea |
| | | | | (Chilean rose |
| | | | | tarantula) |
| 63 | PaTX1 | YCQKWMWTCDSARKCCEGLVCRLWCKKII | P61230.1 | Paraphysa scrofa |
| | | | | (Chilean copper |
| | | | | tarantula) |
| 64 | Magi-5 | GCKLTFWKCKNKKECCGWNACALGICMPR | P83561.2 | Macrothele gigas |
| | | | | (Spider) |
| 65 | HwTx-V | ECRWYLGGCSQDGDCCKHLQCHSNYEWCVWDGTFSK | P0C245.1 | ECRWYLGGCSQDGDCCKH |
| | | | | LQCHSNYEWCVWDGTFSK |
| 66 | VaTx2 | GACRWFLGGCKSTSDCCEHLSCKMGLDYCAWDGTF | P0C245.1 | Psalmopoeus |
| | | | | cambridgei |
| | | | | (Trinidad chevron |
| | | | | tarantula) |
| 67 | SNX482 | GVDKAGCRYMFGGCSVNDDCCPRLGCHSLFSYCAWDL | P56854.1 | Hysterocrates |
| | | TFSD | | gigas (African |
| | | | | tarantula) |
| 68 | PnVIIA | DCTSWFGRCTVNSECCSNSCDQTYCELYAFPSFGA | P56711.2 | Conus pennaceus |
| | | | | (Feathered cone) |
| 69 | PNTx27C4 | IACAPRFSLCNSDKECCKGLRCQSRIANMWPTFCSQ | P83996.2 | Phoneutria |
| | | | | nigriventer |
| | | | | (Brazilian armed |
| | | | | spider) |
| 70 | PRTx27C3 | IACAPRGLLCFRDKECCKGLTCKGRFVNTWPTFCLV | P83892.1 | Phoneutria reidyi |
| | | | | (Brazilian |
| | | | | Amazonian armed |
| | | | | spider) |
| 71 | JZTX-36 | DCRKMFGGCSKHEDCCAHLACKRTFNYCAWDGSFSK | B1P1D7.1 | Chilobrachys |
| | | | | guangxiensis |
| | | | | (Chinese earth |
| | | | | tiger tarantula) |
| 72 | JZTX-38 | ECRWLFGGCEKDSDCCEHLGCRRAKPSWCGWDFTV | B1P1G2.1 | Chilobrachys |
| | | | | guangxiensis |
| | | | | (Chinese earth |
| | | | | tiger tarantula) |
| 73 | JZTX-39 | ECRWLFGGCEKDSDCCEHLGCRRAKPSWCGWDFTF | B1P1G4.1 | Chilobrachys |
| | | | | guangxiensis |
| | | | | (Chinese earth |
| | | | | tiger tarantula) |
| 74 | PRTx26An0C3 | IACAPRFSICNSDKECCKGLRCQSRIANMWPTFCLV | P86418.1 | Phoneutria |
| | | | | nigriventer |
| | | | | (Brazilian armed |
| | | | | spider) |
| 75 | HNTX- | CIGEGVPCDENDPRCCSGLVCLKPTLHGIWYKSYYCY | D2Y253.1 | Haplopelma |
| | XVI | KK | | hainanum (Chinese |
| | | | | bird spider) |
| 76 | HNTX- | DCAGYMRECKEKLCCSGYVCSSRWKWCVLPAPWRR | D2Y240.1 | Haplopelma |
| | VIII | | | hainanum (Chinese |
| | | | | bird spider) |
| 77 | HNTX-IX | ECRWYLGGCSQDGDCCKHLQCHSNYEWCIWDGTFSK | D2Y236.1 | Haplopelma |
| | | | | hainanum (Chinese |
| | | | | bird spider) |
| 78 | F5- | ECKKLFGGCTTSSECCAHLGCKQKWPFYCAWDWSF | P0CH51.1 | Chilobrachys |
| | 21.66 | | | guangxiensis |
| | | | | (Chinese earth |
| | | | | tiger tarantula) |
| 79 | Hm-2 | GCIPSFGECAWFSGESCCTGICKWVFFTSKFMCRRVW | P85506.1 | Heriaeus melloteei |
| | | GKD | | (Crab spider) |
| 80 | HdCa | SEKDCIKHLQRCRENKDCCSKKCSRRGTNPEKRCR | B8QG00.1 | Hadrurus gertschi |
| | | | | (Scorpion) |
| 81 | ProTx-2 | YCQKWMWTCDSERKCCEGMVCRLWCKKKLW | P83476.1 | Thrixopelma |
| | | | | pruriens (Peruvian |
| | | | | green velvet |
| | | | | tarantula) |
| 82 | JzTx-V | YCQKWMWTCDSKRACCEGLRCKLWCRKIIG | Q2PAY4.1 | Chilobrachys |
| | | | | guangxiensis |
| | | | | (Chinese earth |
| | | | | tiger tarantula) |
| 83 | HpTX2 | DDCGKLFSGCDTNADCCEGYVCRLWCKLDW | P58426.1 | Heteropoda |
| | | | | venatoria (Brown |
| | | | | huntsman spider) |
| 84 | GsAF_II | YCQKWMWTCDEERKCCEGLVCRLWCKKKIEW | P61409.2 | Grammostola rosea |
| | | | | (Chilean rose |
| | | | | tarantula) |
| | | | | (Grammostola |
| | | | | spatulata) |
| 85 | MrvIB | ACSKKWEYCIVPILGFVYCCPGLICGPFVCV | AAB34194.1 | Conus marmoreus |
| | | | | (Marble cone) |
| 86 | GsMTx-2 | YCQKWMWTCDEERKCCEGLVCRLWCKRIINM | P60273.1 | Grammostola rosea |
| | | | | (Chilean rose |
| | | | | tarantula) |
| 87 | VSTX2 | YCQKWMWTCDEERKCCEGLVCRLWCKKKIEEG | P0C2P4.1 | Grammostola rosea |
| | | | | (Chilean rose |
| | | | | tarantula) |
| 88 | JZTX-2 | GCGTMWSPCSTEKPCCDNFSCQPAIKWCIWSP | B1P189.1 | Chilobrachys |
| | | | | guangxiensis |
| | | | | (Chinese earth |
| | | | | tiger tarantula) |
| 89 | VaTx3 | ECRWYLGGCKEDSECCEHLQCHSYWEWCLWDGSF | P0C246.1 | Psalmopoeus |
| | | | | cambridgei |
| | | | | (Trinidad chevron |
| | | | | tarantula) |
| 90 | CcoTx3 | GVDKEGCRKLLGGCTIDDDCCPHLGCNKKYWHCGWDG | P84509.1 | Ceratogyrus |
| | | TF | | marshalli |
| | | | | (Straighthorned |
| | | | | baboon tarantula) |
| 91 | JZTX-IV | ECTKFLGGCSEDSECCPHLGCKDVLYYCAWDGTFGK | P0CH56.1 | Chilobrachys |
| | | | | guangxiensis |
| | | | | (Chinese earth |
| | | | | tiger tarantula) |
| 92 | JzTx-IX | ECTKLLGGCTKDSECCPHLGCRKKWPYHCGWDGTF | B1P1F5.2 | Chilobrachys |
| | | | | guangxiensis |
| | | | | (Chinese earth |
| | | | | tiger tarantula) |
| 93 | AcTx- | SPTCIPSGQPCPYNENCCSQSCTFKENENGNTVKRCD | P56207.1 | Hadronyche versuta |
| | Hv1 | | | (Blue mountains |
| | | | | funnel-web spider) |
| | | | | (Atrax versutus) |
| 94 | JZTX-34 | ACREWLGGCSKDADCCAHLECRKKWPYHCVWDWTV | B1P1F7.1 | Chilobrachys |
| | | | | guangxiensis |
| | | | | (Chinese earth |
| | | | | tiger tarantula) |
| 95 | Omega- | SVCIPSGQPCPYNEHCCSGSCTYKENENGNTVQRCD | P83580.2 | Atrax robustus |
| | AcTx- | | | |
| | Ar1a | | | |
| 96 | Omega- | SSTCIPSGQPCPYNENCCSQSCTYKENENGNTVKRCD | P81595 | Hadronyche versuta |
| | hexatoxin- | | | (Blue mountains |
| | Hv1b | | | funnel-web spider) |
| 97 | Omega- | STCTPTDQPCPYHESCCSGSCTYKANENGNQVKRCD | P0C2L4.1 | Hadronyche |
| | AcTx- | | | formidabilis |
| | Hi1a | | | (Northern tree |
| | | | | funnel-web spider) |
| 98 | Omega- | SPTCIRSGQPCPYNENCCSQSCTFKTNENGNTVKRCD | P0C2L4.1 | Hadronyche |
| | AcTx- | | | formidabilis |
| | Hf1a | | | (Northern tree |
| | | | | funnel-web spider) |
| 99 | Omega- | SPTCIPTGQPCPYNENCCSQSCTYKANENGNQVKRCD | P0C2L6.1 | Hadronyche infensa |
| | AcTx- | | | (Fraser island |
| | Hi1b | | | funnel-web spider) |
| 100 | Omega- | SSTCIRTDQPCPYNESCCSGSCTYKANENGNQVKRCD | P0C2L7.1 | Hadronyche infensa |
| | AcTx- | | | (Fraser island |
| | Hi1c | | | funnel-web spider) |
| 101 | Omega- | SSTCIPSGQPCPYNENCCSQSCTFKENENGNTVKRCD | P81596.1 | Hadronyche versuta |
| | AcTx- | | | (Blue mountains |
| | Hv1c | | | funnel-web spider) |
| 102 | Omega- | SPTCIPSGQPCPYNENCCSKSCTYKENENGNTVQRCD | P81597.1 | Hadronyche versuta |
| | AcTx- | | | (Blue mountains |
| | Hv1d | | | funnel-web spider) |
| 103 | Omega- | SPTCIPSGQPCPYNENCCSQSCTYKENENGNTVKRCD | P81598.1 | Hadronyche versuta |
| | AcTx- | | | (Blue mountains |
| | Hv1e | | | funnel-web spider) |
| 104 | magi-1 | CMGYDIHCTDRLPCCFGLECVKTSGYWWYKKTYCRRK | P83557.1 | Macrothele gigas |
| | | S | | (Spider) |
| 105 | Omega- | SPVCTPSGQPCQPNTQPCCNNAEEEQTINCNGNTVYR | P83588.1 | Missulena bradleyi |
| | MSTX- | CA | | (Eastern mouse |
| | Mb1a | | | spider) |
| 106 | F3- | SPVCTP5GQPCQPNTQPCCNNAEEEQTINCNGNTVYR | P0CH70.1 | Haplopelma |
| | 24.71 | CA | | hainanum (Chinese |
| | | | | bird spider) |
| 107 | JzTx- | YCQKWMWTCDSERKCCEGYVCELWCKYNL | P0C5X7.2 | Chilobrachys |
| | XII | | | guangxiensis |
| | | | | (Chinese earth |
| | | | | tiger tarantula) |
| 108 | JZTX- | ACGQFWWKCGEGKPPCCANFACKIGLYLCIWSP | B1P1B7.1 | Chilobrachys |
| | 1.2 | | | guangxiensis |
| | | | | (Chinese earth |
| | | | | tiger tarantula) |
| 109 | GrTx- | DCVRFWGKCSQTSDCCPHLACKSKWPRNICVWDGSV | P60590.2 | Grammostola rosea |
| | SIA | | | (Chilean rose |
| | | | | tarantula) |
| 110 | JZTX-35 | DCRALYGGCTKDEDCCKHLACRRTLPTYCAWDLTFP | B1P1F9.1 | Chilobrachys |
| | | | | guangxiensis |
| | | | | (Chinese earth |
| | | | | tiger tarantula) |

In some embodiments, a wild-type toxin sequence can be a predicted wild-type toxin sequence. In some embodiments, a predicted wild-type toxin sequence is identified in public protein databases. In some embodiments, a sequence element found in a wild-type toxin sequence is identified by isolating an amino acid sequence delineated by six conserved cysteine residues that form disulfide bridges in an ICK motif of a wild-type toxin sequence. In some embodiments, a known amino acid sequence of a wild-type toxin sequence can be used as a template to align amino acid sequences from public protein databases and identify predicted wild type-toxin sequences. In one example, the amino acid sequence of the Peruvian green velvet tarantula (Thrixopelma pruriens) is used as a template to identify predicted wild-type toxin sequences using basic local alignment search tools in public protein databases.

In some embodiments, a polypeptide component has an amino acid sequence that is substantially identical to a sequence set forth in Table 2. In some embodiments, a polypeptide component has an amino acid sequence that is substantially identical to a sequence set forth in Table 2 and an activating or inhibiting effect on Hv1 as set forth in Table 2B. In some embodiments, a polypeptide component has an amino acid sequence that is encoded by a nucleotide sequence that is substantially identical to a sequence set forth in Table 2C.

TABLE 2A

| SEQ | | |
|---|---|---|
| ID | | |
| NO: | Name | Sequence |
| 115 | A1 | DCAGYMRECKKDKECCGWNICNRKH |
| | | KWCKYKLW |
| 116 | A2 | GCQMTFWKCNALDHNCCHGYAACGC |
| | | KKIIVSARIA |
| 117 | A4 | GGCLPHNRFCNPSNDQCCKSANLVC |
| | | RLWCKKKIEGDP |
| 118 | A6, G2 | GCKGFGDSCADSDDCCETFHCKWVF |
| | | FTSKFMCRRVWGKD |
| 119 | B1 | GCLGDKCADSDDCCETFHCKWVFFT |
| | | SKFMCRRVWGKD |
| 120 | B2 | SPTCIPSGQPCADSDDCCETFHCKW |
| | | VFFTSKFMCRRVWGKD |
| 121 | B3 | DEDCQPPGNFCXNTSDCCEHLXCPT |
| | | TPRFPYLCQYXMG |
| 122 | B4 | GACRWFLGGCTPEKNDCCQRLYCGP |
| | | FVCV |
| 123 | B5 | SPVCTPSGQPCRENKDCCSKKCKTT |
| | | GIVKLCRW |
| 124 | B6 | ACSKKWEYCTKDSECCPHLGCWKRR |
| | | RSFEVCVPKTPKT |
| 125 | C1 | RCIEEGKWCTKDEDCCKHLACNRKH |
| | | KWCKYKLW |
| 126 | C2, F2 | SPTCIRSGQPCADSDDCCETFHCKW |
| | | VFFTSKFMCRRVWGKD |
| 127 | C3 | STCTPTDQPCADSDDCCETFHCKWV |
| | | FFTSKFMCRRWGKD |
| 128 | C5 | GCKWYLGDCADSDDCCETFHCKWVF |
| | | FTSKFMCRRVWGKD |
| 129 | C6, D5 | SSTCIPSGQPCADSDDCCETFHCKW |
| | | VFFTSKFMCRRVWGKD |
| 130 | D3 | ACSKKWEYCKEKLCCSGYVCKRRGT |
| | | NIEKRCRG |
| 131 | D4 | ACGQFWWKCTSDSDCCPNWVCRLWC |
| | | KYKL |
| 132 | D6, E2 | CRYWLGGCSQDGDCCKHLQCSPRWG |
| | | WCIYSTRGGR |
| 133 | E1 | DCGTIWHYCTPEKNDCCQRLYCSPR |
| | | WRLVHL |
| 134 | E3 | IACAPRFSICDPKNDKCCPMRVCSD |
| | | KHKWCKWKL |
| 135 | E4 | SSTCIPSGQPCRENKDCCSKKCSDK |
| | | HKWCKWKLG |
| 136 | E5 | DGECGGFWWKCKNSNDCCKDLVCKE |
| | | KWPITYKICVWDRTF |
| 137 | E6 | IACAPRFSLCDTSKDCCEGYVCNRK |
| | | HKWCKYKLW |
| 138 | F4 | ECKGFGKSCADSDDCCETFHCKWVF |
| | | FTSKFMCRRWGKD |
| 139 | F5 | SPVCTPSGQPCADSDDCCETFHCKW |
| | | VFFTSKFMCRRVWGKD |
| 140 | F6 | DDCGGLFSGCTPGKNECCPNRVCKI |
| | | GLYLCIWS |
| 141 | G1 | GCLGDKCADSDDCCETFHCKWVFFT |
| | | SKFMCRRWGKD |
| 142 | G3 | CRYLFGGCAWFSGESCCTGICSPRW |
| | | GWCIYSTRGGR |
| 143 | G4 | GDCLPHLKLCNPNDDKCCRPKLKCS |
| | | RRGTNPEKRCR |
| 144 | G6 | DDCGTLFSGCPYSKYCCSGSCKRRG |
| | | TNIEKRCR |
| 145 | H4 | AAEGCLCDRCXHSGDCCEDFHCTCE |
| | | FFNM |

TABLE 2B

| SEQ | | | Activator/ |
|---|---|---|---|
| ID NO: | Name | Sequence | Inhibitor |
| 118 | A6 | GCKGFGDSCADSDDCCET | ACTIVATOR |
| | | FHCKWVFFTSKFMCRRVW | |
| | | GKD | |
| 119 | B1 | GCLGDKCADSDDCCETFH | ACTIVATOR |
| | | CKWVFFTSKFMCRRVWGK | |
| | | D | |
| 128 | C5 | GCKWYLGDCADSDDCCET | ACTIVATOR |
| | | FHCKWVFFTSKFMCRRVW | |
| | | GKD | |
| 129 | C6 | SSTCIPSGQPCADSDDCC | INHIBITOR |
| | | ETFHCKWVFFD | |
| 129 | D5 | SSTCIPSGQPCADSDDCC | INHIBITOR |
| | | ETFHCKWVFFTSKFMCRR | |
| | | VWGKD | |
| 126 | F2 | SPTCIRSGQPCADSDDCC | ACTIVATOR |
| | | ETFHCKWVFFTSKFMCRR | |
| | | VWGKD | |
| 118 | G2 | GCKGFGDSCADSDDCCET | ACTIVATOR |
| | | FHCKWVFFTSKFMCRRVW | |
| | | GKD | |

TABLE 2C

| SEQ | | |
|---|---|---|
| ID | | |
| NO: | Name | Nucleotide Sequences |
| 146 | A1 | GATTGCGCGGGCTATATGCGCGAAT |
| | | GTAAAAAAGATAAAGAATGCTGCGG |
| | | CTGGAACATTTGCAACCGCAAACAT |
| | | AAATGGTGCAAATATAAACTGTGG |
| 147 | A2 | GGCTGCCAAATGACCTTTTGGAAAT |
| | | GTAACGCGCTGGATCACAACTGCTG |
| | | CCATGGCTATGCCGCCTGTGGATGC |
| | | AAAAAAATTATTGTATCCGCGAGAA |
| | | TCGCG |
| 148 | A4 | GGCGGCTGCCTGCCGCATAACCGCT |
| | | TTTGTAACCCGAGCAACGATCAGTG |
| | | CTGCAAAAGCGCGAACCTGGTGTGC |
| | | CGCCTGTGGTGCAAAAAAAAAATTG |
| | | AAGGGGATCCG |
| 149 | A6, | GGCTGCAAAGGCTTTGGCGATAGCT |
| | G2 | GTGCGGATAGCGATGATTGCTGCGA |
| | | AACCTTTCATTGCAAATGGGTGTTT |
| | | TTTACCAGCAAATTTATGTGCCGCC |
| | | GCGTGTGGGGCAAAGAT |
| 150 | B1 | GGCTGCCTGGGCGATAAATGTGCGG |
| | | ATAGCGATGATTGCTGCGAAACCTT |
| | | TCATTGCAAATGGGTGTTTTTTACC |
| | | AGCAAATTTATGTGCCGCCGCGTGT |
| | | GGGGCAAAGAT |
| 151 | B2 | AGCCCGACCTGCATTCCGAGCGGCC |
| | | AGCCGTGTGCGGATAGCGATGATTG |
| | | CTGCGAAACCTTTCATTGCAAATGG |
| | | GTGTTTTTTACCAGCAAATTTATGT |
| | | GCCGCCGCGTGTGGGGCAAAGATGG |
| | | AT |
| 152 | B3 | GACGAAGATTGCCAACCGCCGGGCA |
| | | ACTTTTGTANCAACACCAGCGATTG |
| | | CTGCGAACATCTGNNCTGCCCGACC |
| | | ACCCCCCGCTTTCCCTATCTGTGCC |
| | | AATACCNCATGGGA |
| 153 | B4 | AGGCGCGTGCCGCTGGTTTCTGGGC |
| | | GGCTGTACCCCGGAAAAAAACGATT |
| | | GCTGCCAGCGCCTGTATTGCGGCCC |
| | | GTTTGTGTGCGTG |
| 154 | B5 | AGCCCGGTGTGCACCCCGAGCGGCC |
| | | AGCCGTGTCGCGAAAACAAAGATTG |
| | | CTGCAGCAAAAAATGCAAAACC |
| | | ACCGGCATTGTGAAACTGTGCCGCT |
| | | GG |
| 155 | B6 | GCGTGCAGCAAAAAATGGGAATATT |
| | | GTACCAAAGATAGCGAATGCTGCCC |
| | | GCATCTGGGCTGCTGGAAACGCCGC |
| | | CGCAGCTTTGAAGTGTGCGTGCCGA |
| | | AAACCCCGAAAACC |
| 156 | Cl | CGCTGCATTGAAGAAGGCAAATGGT |
| | | GTACCAAAGATGAAGATTGCTGCAA |
| | | ACATCTGGCGTGCAACCGCAAACAT |
| | | AAATGGTGCAAATATAAACTGTGG |
| 157 | C2, | AGCCCGACCTGCATTCGCAGCGGCC |
| | F2 | AGCCGTGTGCGGATAGCGATGATTG |
| | | CTGCGAAACCTTTCATTGCAAATGG |
| | | GTGTTTTTTACCAGCAAATTTATGT |
| | | GCCGCCGCGTGTGGGGCAAAGAT |
| 158 | C3 | AGCACCTGCACCCCGACCGATCAGC |
| | | CGTGTGCGGATAGCGATGATTGCTG |
| | | CGAAACCTTTCATTGCAAATGGGTG |
| | | TTTTTTACCAGCAAATTTATGTGCC |
| | | GCCGCGTGTGGGGCAAAGAT |
| 159 | C4 | AAATGCCGCTGGCTGTTTGGCGGGG |
| | | TACCCCGGGCAAAAACGAATGCTGG |
| | | CCGAACTATGCGTGCCATAGCTATT |
| | | GGGAATGGGGCCTGTGGGATGGCAG |
| | | CTTTGGATCCG |
| 160 | C5 | GGCTGCAAATGGTATCTGGGCGATT |
| | | GTGCGGATAGCGATGATTGCTGCGA |
| | | AACCTTTCATTGCAAATGGGTGTTT |
| | | TTTACCAGCAAATTTATGTGCCGCC |
| | | GCGTGTGGGGCAAAGAT |
| 161 | C6, | AGCAGCACCTGCATTCCGAGCGGCC |
| | D5 | AGCCGTGTGCGGATAGCGATGATTG |
| | | CTGCGAAACCTTTCATTGCAAA |
| | | TGGGTGTTTTTTACCAGCAAATTTA |
| | | TGTGCCGCCGCGTGTGGGGCAAAGA |
| | | T |
| 162 | D3 | GCGTGCAGCAAAAAATGGGAATATT |
| | | GTAAAGAAAAACTGTGCTGCAGCGG |
| | | CTATGTGTGCAAACGCCGCGGCACC |
| | | AACATTGAAAAACGCTGCCGCGGA |
| 163 | D4 | GCGTGCGGCCAGTTTTGGTGGAAAT |
| | | GTACCAGCGATAGCGATTGCTGCCC |
| | | GAACTGGGTGTGCCGCCTGTGG |
| | | TGCAAATATAAACTG |
| 164 | D6, | TGCCGCTATTGGCTGGGCGGCTGTA |
| | E2 | GCCAGGATGGCGATTGCTGCAAACA |
| | | TCTGCAGTGCAGCCCGCGCTGGGGC |
| | | TGGTGCATTTATAGCACCCGCGGCG |
| | | GCCGC |
| 165 | El | GATTGCGGCACCATTTGGCATTATT |
| | | GTACCCCGGAAAAAAACGATTGCTG |
| | | CCAGCGCCTGTATTGCAGCCCGCGC |
| | | TGGAGGCTGGTGCATTTA |
| 166 | E3 | ATTGCGTGCGCGCCGCGCTTTAGCA |
| | | TTTGTGATCCGAAAAACGATAAATG |
| | | CTGCCCGAACCGCGTGTGCAGC |
| | | GATAAACATAAATGGTGCAAATGGA |
| | | AACTG |
| 167 | E4 | AGCAGCACCTGCATTCCGAGCGGCC |
| | | AGCCGTGTCGCGAAAACAAAGATTG |
| | | CTGCAGCAAAAAATGCAGCGATAAA |
| | | CATAAATGGTGCAAATGGAAACTGG |
| | | GA |
| 168 | E5 | GATGGCGAATGCGGCGGCTTTTGGT |
| | | GGAAATGTAAAAACAGCAACGATTG |
| | | CTGCAAAGATCTGGTGTGCAAAGAA |
| | | AAATGGCCGATTACCTATAAAATTT |
| | | GCGTGTGGGATCGCACCTTT |
| 169 | E6 | ATTGCGTGCGCGCCGCGCTTTAGCC |
| | | TGTGTGATACCAGCAAAGATTGCTG |
| | | CGAAGGCTATGTGTGCAACCGC |
| | | AAACATAAATGGTGCAAATATAAAC |
| | | TGTGG |
| 170 | F4 | GAATGCAAAGGCTTTGGCAAAAGCT |
| | | GTGCGGATAGCGATGATTGCTGCGA |
| | | AACCTTTCATTGCAAATGGGTGTTT |
| | | TTTACCAGCAAATTTATGTGCCGCC |
| | | GCGTGTGGGGCAAAGAT |
| 171 | F5 | AGCCCGGTGTGCACCCCGAGCGGCC |
| | | AGCCGTGTGCGGATAGCGATGATTG |
| | | CTGCGAAACCTTTCATTGCAAATGG |
| | | GTGTTTTTTACCAGCAAATTTATGT |
| | | GCCGCCGCGTGTGGGGCAAAGAT |
| 172 | F6 | GATGATTGCGGCGGCCTGTTTAGCG |
| | | GCTGTACCCCGGGCAAAAACGAATG |
| | | CTGCCCGAACCGCGTGTGCAAA |
| | | ATTGGCCTGTATCTGTGCATTTGGA |
| | | GCCCG |
| 173 | G1 | GGCTGCCTGGGCGATAAATGTGCGG |
| | | ATAGCGATGATTGCTGCGAAACCTT |
| | | TCATTGCAAATGGGTGTTTTTTACC |
| | | AGCAAATTTATGTGCCGCCGCGTGT |
| | | GGGGCAAAGAT |
| 174 | G3 | TGCCGCTATCTGTTTGGCGGCTGTG |
| | | CGTGGTTTAGCGGCGAAAGCTGCTG |
| | | CACCGGCATTTGCAGCCCGCGCTGG |
| | | GGCTGGTGCATTTATAGCACCCGCG |
| | | GCGGCCGC |
| 175 | G4 | GGCGATTGCCTGCCGCATCTGAAAC |
| | | TGTGTAACCCGAACGATGATAAATG |
| | | CTGCCGCCCGAAACTGAAATGCAGC |
| | | CGCCGCGGCACCAACCCGGAAAAAC |
| | | GCTGCCGC |
| 176 | G6 | GATGATTGCGGCACCCTGTTTAGCG |
| | | GCTGTCCGTATAGCAAATATTGCTG |
| | | CAGCGGCAGCTGCAAACGCCGCGGC |
| | | ACCAACATTGAAAAACGCTGCCGC |
| 177 | H4 | GCTGCCTGTGCGATAGATGTGTNCA |
| | | TAGCGGTGATTGTTGCGAAGACTTT |
| | | CATTGCACCTGCGAGTTTTTTAACA |
| | | TGTAATTTATG |

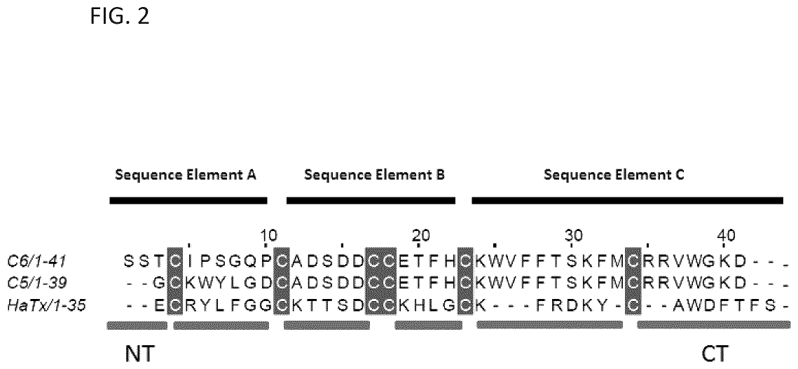

In some embodiments, a polypeptide component is composed of one or more polypeptide elements, each of which has an amino acid sequence that is substantially identical to a reference sequence element A, B, or C as set forth in Table 3A. In some embodiments, one or more A, B, C reference sequence elements is or comprises a wild type toxin sequence element. In some embodiments, a polypeptide component has an amino acid sequence that comprises or consists of a single sequence element corresponding to an A reference sequence element, a single sequence element corresponding to a B reference sequence element, and a single sequence element corresponding to a C reference sequence element. In some such embodiments, the single sequence elements are arranged in a linear order as follows: A-B-C. Examples of Hv1 modulating agents having A-B-C sequence elements are depicted in FIG. 2 and Table 2. Examples of nucleotide sequences encoding polypeptide sequence elements A, B, and C are set forth in Table 3B3.

TABLE 3A

| SEQ | | SEQ | | SEQ | |
|---|---|---|---|---|---|
| ID | | ID | | ID | |
| NO: | Element A | NO: | Element B | NO: | Element C |
| 178 | AACLGMFESC | 273 | ADSDDCCETFHC | 377 | ALGICMPR |
| 179 | ACGQFWWKC | 274 | AWFSGESCCTGIC | 378 | ATTGRFRYLCKYQI |
| 180 | ACKGVFDAC | 275 | DEERKCCEGLVC | 379 | DRRDQWCKWNPW |
| 181 | ACREWLGGC | 276 | DENDPRCCSGLVC | 380 | ELWCKYNL |
| 182 | ADCGWLFHSC | 277 | DGKSTFCCSGFNC | 381 | ERSSPWCKIDIW |
| 183 | CAAEGIPC | 278 | DGKSTFCCSGYNC | 382 | HLWCKYK |
| 184 | CGGWMAKC | 279 | DPDNDKCCEGYKC | 383 | HSLFSYCAWDLTFSD |
| 185 | CIGEGVPC | 280 | DPKNDKCCKNYTC | 384 | HSNYEWCIWDGTFSK |
| 186 | CMGYDIHC | 281 | DPKNDKCCPNRVC | 385 | HSNYEWCVWDGT |
| 187 | DCAGYMREC | 282 | DPNNDKCCPNREC | 386 | HSRWDWCIWDGTF |
| 188 | DCGTIWHYC | 283 | DPNNDKCCPNRVC | 387 | HSYWEWCLWDGSF |
| 189 | DCLGFLWKC | 284 | DPNPVKDLPCCSGLAC | 388 | ICSGXNWK |
| 190 | DCLGLFWIC | 285 | DSARKCCEGLVC | 389 | ISTKVNYYCRPDRGP |
| 191 | DCLGWFKGC | 286 | DSERKCCEDMVC | 390 | KAFVLHCYRN |
| 192 | DCLGWFKSC | 287 | DSERKCCEGMVC | 391 | KDVLYYCAWDGTF |
| 193 | DCRALYGGC | 288 | DSERKCCEGYVC | 392 | KEKWPITYKICVWDRTF |
| 194 | DCRKMFGGC | 289 | DSKRACCEGLRC | 393 | KELSIWDSRCL |
| 195 | DCTRMFGAC | 290 | DSKRKCCEDMVC | 394 | KFRDKYCAWDFTFS |
| 196 | DCVRFWGKC | 291 | DSNADCCEGYVC | 395 | KGPSPKQKKCTRP |
| 197 | DDCGGLFSGC | 292 | DSTLDCCKHLSC | 396 | KGRFVNTWPTFCLV |
| 198 | DDCGKLFSGC | 293 | DTNADCCEGYVC | 397 | KIGLYLCIWSP |
| 199 | DDCGTLFSGC | 294 | DTSKDCCEGYVC | 398 | KLWCRKIIG |
| 200 | DDCLGMFSSC | 295 | DYNNGCCSGYVC | 399 | KLWCRYERTW |
| 201 | DDDCGWIMDDC | 296 | EKDEHCCEHLGC | 400 | KMGLDYCAWDGTF |
| 202 | DGECGGFWWKC | 297 | EKDSDCCEHLGC | 401 | KMGLYYCAWDGTF |
| 203 | ECGKFMWKC | 298 | ESNADCCENWAC | 402 | KPTLKYCAWDGT |
| 204 | ECGTLFSGC | 299 | FRDKECCKGLTC | 403 | KPTLKYCAWDGTF |
| 205 | ECKGFGKSC | 300 | GAGKPTCCSGYDC | 404 | KPTSKYCAWDGTI |
| 206 | ECKKLFGGC | 301 | GEGKPPCCANFAC | 405 | KQKWPFYCAWDWSF |
| 207 | ECKWYLGDC | 302 | GRGKPPCCKGYAC | 406 | KRRGTNAEKRCR |
| 208 | ECKYLWGTC | 303 | GSGKPACCPKYVC | 407 | KRRGTNIEKRCR |
| 209 | ECLEIFKAC | 304 | GTDQSECCEGWKC | 408 | KRTFNYCAWDGSFSK |
| 210 | ECLGFGKGC | 305 | HPGQPPCCSGLAC | 409 | KSKWPRNICVWDGSV |
| 211 | ECRKMFGGC | 306 | KADNDCCGKKC | 410 | KTTGIVKLCRW |
| 212 | ECRWLFGGC | 307 | KAHEDCCEHLRC | 411 | KWVFFTSKFMCRRVWGKD |
| 213 | ECRWYLGGC | 308 | KEDSECCEHLQC | 412 | LKPTLHGIWYKHHYCYTQ |
| 214 | ECRYFWGEC | 309 | KEKLCCSGYVC | 413 | LKPTLHGIWYKSYYCYKK |
| 215 | ECRYLFGGC | 310 | KENKDCCSKKC | 414 | NGNTVYRCA |
| 216 | ECRYWLGGC | 311 | KKDKECCGWNIC | 415 | NKKHGWCGWDGTF |
| 217 | ECRYWLGTC | 312 | KNKKECCGWNAC | 416 | NKKYWHCGWDGTF |
| 218 | ECTKFLGGC | 313 | KNSNDCCKDLVC | 417 | NRKHKWCKYKLW |
| 219 | ECTKLLGGC | 314 | KSDENCCKKFKC | 418 | NRRDKWCKYKLW |
| 220 | EDCIPKWKGC | 315 | KSTSDCCEHLSC | 419 | NSRDKWCKVLL |
| 221 | EGECGGFWWKC | 316 | KTTADCCKHLAC | 420 | QLWCKKRL |
| 222 | GACRWFLGGC | 37 | KTTADCCKHLGC | 421 | QPAIKWCIWSP |
| 223 | GCANAYKSC | 318 | KTTSDCCKHLGC | 422 | QSRIANMWPTFCLV |
| 224 | GCGGLMAGC | 319 | NALSGPRCCSGLKC | 423 | QSRIANMWPTFCSQ |
| 225 | GCGGLMDGC | 320 | NDEMVCCEHLVC | 424 | RKKWPYHCGWDGTF |
| 226 | GCGTMWSPC | 321 | NGPHTCCWGYNGYKKAC | 425 | RKKWPYHCVWDWTV |
| 227 | GCIPSFGEC | 322 | NPDNDKCCEGRKC | 426 | RLWCKKII |
| 228 | GCKGFGDSC | 323 | NPNDDKCCRPKLKC | 427 | RLWCKKKIEEG |
| 229 | GCKLTFWKC | 324 | NPSNDKCCRPNLVC | 428 | RLWCKKKIEW |
| 230 | GCLEFWWKC | 325 | NPSNDQCCKSANLVC | 429 | RLWCKKKLW |
| 231 | GCLGDKC | 326 | NPSNDQCCKSSKLVC | 430 | RLWCKKRL |
| 232 | GCNRKNKKC | 327 | NPSNDQCCKSSNLVC | 431 | RLWCKLDW |
| 233 | GCQKFFWTC | 328 | NSDADCCRYGERC | 432 | RLWCKRIINM |
| 234 | GDCLPHLKLC | 329 | NSDKECCKGLRC | 433 | RLWCKYKL |
| 235 | GDCLPHLKRC | 330 | NYMDDKCCPGYKC | 434 | RRAKPSWCGWDFTF |
| 236 | GGCLPHNRFC | 331 | PKKAPCCGRLEC | 435 | RRAKPSWCGWDFTV |
| 237 | GVDKAGCRYMFGGC | 332 | PYHESCCSGSC | 436 | RRTLPTYCAWDLTFP |
| 238 | GVDKEGCRKLLGGC | 333 | PYNEHCCSGSC | 437 | RSDGKYCAWDGTF |
| 239 | IACAPRFSIC | 334 | PYNENCCSKSC | 438 | RSDWKYCAWDGTFS |
| 240 | IACAPRFSLC | 335 | PYNENCCSQSC | 439 | RSRDQWCKYKLW |
| 241 | IACAPRGLLC | 336 | PYNESCCSGSC | 440 | RVRDQWCKYKLW |
| 242 | KCLPPGKPC | 337 | PYSKYCCSGSC | 441 | SDKHKWCKWKL |
| 243 | LCSREGEFC | 338 | QPNTQPCCNNAEEEQTINC | 442 | SHNKCT |
| 244 | QCGEFMWKC | 339 | RENKDCCSKKC | 443 | SKLFKLCNFSF |
| 245 | RCIEEGKWC | 340 | RRDSDCCPHLGC | 444 | SKTGFVKNICKYEM |
| 246 | SAVCIPSGQPC | 341 | SAGQTCCKHLVC | 445 | SKTWGWCAVEAP |
| 247 | SCKLTFWRC | 342 | SEDSECCPHLGC | 446 | SPKHGWCVWDWTFRK |
| 248 | SECRWFMGGC | 343 | SKDADCCAHLEC | 447 | SPKWGLCNFPMP |
| 249 | SEKDCIKHLQRC | 344 | SKHEDCCAHLAC | 448 | SPRWGWCIYSTRGGR |
| 250 | SFCIPFKPC | 345 | SKTGDCCSHLSC | 449 | SPTWKWCVLKSPGRR |
| 251 | SPTCIPSGQPC | 346 | SQDGDCCKHLQC | 450 | SPTWKWCVYARP |
| 252 | SPTCIPTGQPC | 347 | SQTSDCCPHLAC | 451 | SQHRLCSVKA |
| 253 | SPTCIRSGQPC | 348 | SSDKPCCSGYYC | 452 | SRKDKWCKYQI |
| 254 | SPVCTPSGQPC | 349 | SSTSDCCKHLSC | 453 | SRKHRWCKYEI |
| 255 | SSTCIPSGQPC | 350 | STEKPCCDNFSC | 454 | SRKTRWCKYQI |
| 256 | SSTCIRTDQPC | 351 | STHADCCEGFIC | 455 | SRQLCKYVIDW |
| 257 | STCTPTDQPC | 352 | SVDSDCCAHLGC | 456 | SRRDRWCKYDL |
| 258 | SVCIPSGQPC | 353 | SVHSDCCAHLGC | 457 | SRRDRWCKYYL |
| 259 | TCRYLFGGC | 354 | SVNDDCCPRLGC | 458 | SRRGTNPEKRCR |
| 260 | TCYDIGELC | 355 | TDRLPCCFGLEC | 459 | SRRHGWCVWDGTFS |
| 261 | VCRGYGLPC | 356 | TIDDDCCPHLGC | 460 | SRTWKWCVLAGPW |
| 262 | YCQKWLWTC | 357 | TKDEDCCKHLAC | 461 | SSKHKWCKVYL |
| 263 | YCQKWMWTC | 358 | TKDSECCPHLGC | 462 | SSRWKWCVLASPF |
| 264 | CKQADEPC | 359 | TPEKNDCCQRLYC | 463 | SSRWKWCVLPAPW |
| 265 | ACRKKWEYC | 360 | TPGKNECCPNRVC | 464 | TFKENENGNTVKRCD |
| 266 | DDDCEPPGNFC | 361 | TPGKNECCPNYAC | 465 | TFKTNENGNTVKRCD |
| 267 | VKPCRKEGQLC | 362 | TSDSDCCPNWVC | 466 | TGLCIPP |
| 268 | WCKQSGEMC | 363 | TTSSECCAHLGC | 467 | TRFNVCGK |
| 269 | CLSGGEVC | 364 | VNRHGDCCEGLEC | 468 | TWPTEICID |
| 270 | GKPCHEEGCQL | 365 | VPGKNECCSGYAC | 469 | TYKANENGNQVKRCD |
| 271 | CIPFLHPC | 366 | YGATQKIPCCGVC | 470 | TYKENENGNTVKRCD |
| 272 | ACSKKWEYC | 367 | YKLRKCCAGFYC | 471 | TYKENENGNTVQRCD |
| | | 368 | DVFSLDCCTGIC | 472 | TYKTNENGNSVQRCD |
| | | 369 | IVPIIGFIYCCPGLIC | 473 | VKTSGYWWYKKTYCRRKS |
| | | 370 | GMIKIGPPCCSGWC | 474 | WKRRRSFEVCVPKTPKT |
| | | 371 | DPIFQNCCRGWNC | 475 | LGVCMW |
| | | 372 | NVLDQNCCDGYC | 476 | FFACA |
| | | 373 | DFLFPKCCNYC | 477 | VLFCV |
| | | 374 | DPFLQNCCLGWNC | 478 | IVFVCT |
| | | 375 | TFFFPDCCNSIC | 479 | ILLFCS |
| | | 376 | IVPILGFVYCCPGLIC | 480 | VFVCI |
| | | | | 481 | AQFICL |
| | | | | 482 | GPFVCV |

TABLE 3B

| SEQ | | SEQ | | SEQ | |
|---|---|---|---|---|---|
| ID | | ID | | ID | |
| NO. | Element A | NO. | Element B | NO. | Element C |
| 483 | GCGGCGTGCCTGG | 578 | GCGGATAGCGATGATTGCTGC | 682 | GCGCTGGGCATTTGCAT |
| | GCATGTTTGAAAGCTGT | | GAAACCTTTCATTGC | | GCCGCGC |
| 484 | GCGTGCGGCCAGT | 579 | GCGTGGTTTAGCGGCGAAAGC | 683 | GCGACCACCGGCCGCTTTCGCT |
| | TTTGGTGGAAATGT | | TGCTGCACCGGCATTTGC | | ATCTGTGCAAATATCAGATT |
| 485 | GCGTGCAAAGGCG | 580 | GATGAAGAACGCAAATGCTGC | 684 | GATCGCCGCGATCAGTG |
| | TGTTTGATGCGTGT | | GAAGGCCTGGTGTGC | | GTGCAAATGGAACCCGTGG |
| 486 | GCGTGCCGCGAAT | 581 | GATGAAAACGATCCGCGCTGC | 685 | GAACTGTGGTGCAAATA |
| | GGCTGGGCGGCTGT | | TGCAGCGGCCTGGTGTGC | | TAACCTG |
| 487 | GCGGATTGCGGCT | 582 | GATGGCAAAAGCACCTTTTGCT | 686 | GAACGCAGCAGCCCGTG |
| | GGCTGTTTCATAGCTGT | | GCAGCGGCTTTAACTGC | | GTGCAAAATTGATATTTGG |
| 488 | TGCGCGGCGGAAG | 583 | GATGGCAAAAGCACCTTTTGCT | 687 | CATCTGTGGTGCAAATAT |
| | GCATTCCGTGT | | GCAGCGGCTATAACTGC | | AAA |
| 489 | TGCGGCGGCTGGA | 584 | GATCCGGATAACGATAAATGCT | 688 | CATAGCCTGTTTAGCTATTGCGC |
| | TGGCGAAATGT | | GCGAAGGCTATAAATGC | | GTGGGATCTGACCTTTAGCGAT |
| 490 | TGCATTGGCGAAGG | 585 | GATCCGAAAAACGATAAATGCT | 689 | CATAGCAACTATGAATGGTGCAT |
| | CGTGCCGTGT | | GCAAAAACTATACCTGC | | TTGGGATGGCACCTTTAGCAAA |
| 491 | TGCATGGGCTATGA | 586 | GATCCGAAAAACGATAAATGCT | 690 | CATAGCAACTATGAATG |
| | TATTCATTGT | | GCCCGAACCGCGTGTGC | | GTGCGTGTGGGATGGCACC |
| 492 | GATTGCGCGGGCT | 587 | GATCCGAACAACGATAAATGCT | 691 | CATAGCCGCTGGGATTG |
| | ATATGCGCGAATGT | | GCCCGAACCGCGAATGC | | GTGCATTTGGGATGGCACCTTT |
| 493 | GATTGCGGCACCAT | 588 | GATCCGAACAACGATAAATGCT | 692 | CATAGCTATTGGGAATG |
| | TTGGCATTATTGT | | GCCCGAACCGCGTGTGC | | GTGCCTGTGGGATGGCAGCTTT |
| 494 | GATTGCCTGGGCTT | 589 | GATCCGAACCCGGTGAAAGAT | 693 | ATTTGCAGCGGCAACTG |
| | TCTGTGGAAATGT | | CTGCCGTGCTGCAGCGGCCTG | | GAAA |
| | | | GCGTGC | | |
| 495 | GATTGCCTGGGCTTT | 590 | GATAGCGCGCGCAAATGCTGC | 694 | ATTAGCACCAAAGTGAACTAT |
| | CTGTGGAAATGT | | GAAGGCCTGGTGTGC | | TATCGCCCGGATCGCGGCCCG |
| 496 | GATTGCCTGGGCTG | 591 | GATAGCGAACGCAAATGCTGC | 695 | AAAGCGTTTGTGCTGCA |
| | GTTTAAAGGCTGT | | GAAGATATGGTGTGC | | TTGCTATCGCAAC |
| 497 | GATTGCCTGGGCTG | 592 | GATAGCGAACGCAAATGCTGC | 696 | AAAGATGTGCTGTATTAT |
| | GTTTAAAAGCTGT | | GAAGGCATGGTGTGC | | TGCGCGTGGGATGGCACCTTT |
| 498 | GATTGCCGCGCGC | 593 | GATAGCGAACGCAAATGCTGC | 697 | AAAGAAAAATGGCCGATTACCTATAA |
| | TGTATGGCGGCTGT | | GAAGGCTATGTGTGC | | AATTTGCGTGTGGGATCGCACCTTT |
| 499 | GATTGCCGCAAAAT | 594 | GATAGCAAACGCGCGTGCTGC | 698 | AAAGAACTGAGCATTTG |
| | GTTTGGCGGCTGT | | GAAGGCCTGCGCTGC | | GGATAGCCGCTGCCTG |
| 500 | GATTGCACCCGCAT | 595 | GATAGCAAACGCAAATGCTGC | 699 | AAATTTCGCGATAAATAT |
| | GTTTGGCGCGTGT | | GAAGATATGGTGTGC | | TGCGCGTGGGATTTTAC |
| | | | | | CTTTAGC |
| 501 | GATTGCGTGCGCTT | 596 | GATAGCAACGCGGATTGCTGC | 700 | AAAGGCCCGAGCCCGAA |
| | TTGGGGCAAATGT | | GAAGGCTATGTGTGC | | ACAGAAAAAATGCACCCGCCCG |
| 502 | GATGATTGCGGCG | 597 | GATAGCACCCTGGATTGCTGC | 701 | AAAGGCCGCTTTGTGAA |
| | GCCTGTTTAGCGGCTGT | | AAACATCTGAGCTGC | | CACCTGGCCGACCTTTTGCCTGGTG |
| 503 | GATGATTGCGGCAA | 598 | GATACCAACGCGGATTGCTGC | 702 | AAAATTGGCCTGTATCTG |
| | ACTGTTTAGCGGCTGT | | GAAGGCTATGTGTGC | | TGCATTTGGAGCCCG |
| 504 | GATGATTGCGGCAC | 599 | GATACCAGCAAAGATTGCTGC | 703 | AAACTGTGGTGCCGCAA |
| | CCTGTTTAGCGGCTGT | | GAAGGCTATGTGTGC | | AATTATTGGC |
| 505 | GATGATTGCCTGGG | 600 | GATTATAACAACGGCTGCTGCA | 704 | AAACTGTGGTGCCGCTA |
| | CATGTTTAGCAGCTGT | | GCGGCTATGTGTGC | | TGAACGCACCTGG |
| 506 | GATGATGATTGCGG | 601 | GAAAAAGATGAACATTGCTGCG | 705 | AAAATGGGCCTGGATTA |
| | CTGGATTATGGATGATTGT | | AACATCTGGGCTGC | | TTGCGCGTGGGATGGCACCTTT |
| 507 | GATGGCGAATGCG | 602 | GAAAAAGATAGCGATTGCTGC | 706 | AAAATGGGCCTGTATTAT |
| | GCGGCTTTTGGTGGAAATGT | | GAACATCTGGGCTGC | | TGCGCGTGGGATGGCACCTTT |
| 508 | GAATGCGGCAAATT | 603 | GAAAGCAACGCGGATTGCTGC | 707 | AAACCGACCCTGAAATAT |
| | TATGTGGAAATGT | | GAAAACTGGGCGTGC | | TGCGCGTGGGATGGCACC |
| 509 | GAATGCGGCACCCT | 604 | TTTCGCGATAAAGAATGCTGCA | 708 | AAACCGACCCTGAAATAT |
| | GTTTAGCGGCTGT | | AAGGCCTGACCTGC | | TGCGCGTGGGATGGCACCTTT |
| 510 | GAATGCAAAGGCTT | 605 | GGCGCGGGCAAACCGACCTGC | 709 | AAACCGACCAGCAAATA |
| | TGGCAAAAGCTGT | | TGCAGCGGCTATGATTGC | | TTGCGCGTGGGATGGCACCATT |
| 511 | GAATGCAAAAAACT | 606 | GGCGAAGGCAAACCGCCGTGC | 710 | AAACAGAAATGGCCGTT |
| | GTTTGGCGGCTGT | | TGCGCGAACTTTGCGTGC | | TTATTGCGCGTGGGATTGGAGCTTT |
| 512 | GAATGCAAATGGTA | 607 | GGCCGCGGCAAACCGCCGTGC | 711 | AAACGCCGCGGCACCAA |
| | TCTGGGCGATTGT | | TGCAAAGGCTATGCGTGC | | CGCGGAAAAACGCTGCCGC |
| 513 | GAATGCAAATATCT | 608 | GGCAGCGGCAAACCGGCGTGC | 712 | AAACGCCGCGGCACCAA |
| | GTGGGGCACCTGT | | TGCCCGAAATATGTGTGC | | CATTGAAAAACGCTGCCGC |
| 514 | GAATGCCTGGAAAT | 609 | GGCACCGATCAGAGCGAATGC | 713 | AAACGCACCTTTAACTAT |
| | TTTTAAAGCGTGT | | TGCGAAGGCTGGAAATGC | | TGCGCGTGGGATGGCAGCTTTAGCAAA |
| 515 | GAATGCCTGGGCTT | 610 | CATCCGGGCCAGCCGCCGTGC | 714 | AAAAGCAAATGGCCGCG |
| | TGGCAAAGGCTGT | | TGCAGCGGCCTGGCGTGC | | CAACATTTGCGTGTGGGATGGCAGCGTG |
| 516 | GAATGCCGCAAAAT | 611 | AAAGCGGATAACGATTGCTGC | 715 | AAAACCACCGGCATTGT |
| | GTTTGGCGGCTGT | | GGCAAAAAATGC | | GAAACTGTGCCGCTGG |
| 517 | GAATGCCGCTGGCT | 612 | AAAGCGCATGAAGATTGCTGC | 716 | AAATGGGTGTTTTTTACCAGCAAATT |
| | GTTTGGCGGTGT | | GAACATCTGCGCTGC | | TATGTGCCGCCGCGTGTGGGGCAAAGAT |
| 518 | GAATGCCGCTGGTA | 613 | AAAGAAGATAGCGAATGCTGC | 717 | CTGAAACCGACCCTGCATGGCATTTG |
| | TCTGGGCGGCTGT | | GAACATCTGCAGTGC | | GTATAAACATCATTATTGCTATACCCAG |
| 519 | GAATGCCGCTATTT | 614 | AAAGAAAAACTGTGCTGCAGC | 718 | CTGAAACCGACCCTGCATGGCATTTGGT |
| | TTGGGGCGAATGT | | GGCTATGTGTGC | | ATAAAAGCTATTATTGCTATAAAAAA |
| 520 | GAATGCCGCTATCT | 615 | AAAGAAAACAAAGATTGCTGCA | 719 | AACGGCAACACCGTGTA |
| | GTTTGGCGGCTGT | | GCAAAAAATGC | | TCGCTGCGCG |
| 521 | GAATGCCGCTATTG | 616 | AAAAAAGATAAAGAATGCTGCG | 720 | AACAAAAAACATGGCTG |
| | GCTGGGCGGCTGT | | GCTGGAACATTTGC | | GTGCGGCTGGGATGGCACCTTT |
| 522 | GAATGCCGCTATTG | 617 | AAAAACAAAAAAGAATGCTGCG | 721 | AACAAAAAATATTGGCAT |
| | GCTGGGCACCTGT | | GCTGGAACGCGTGC | | TGCGGCTGGGATGGCACCTTT |
| 523 | GAATGCACCAAATT | 618 | AAAAACAGCAACGATTGCTGCA | 722 | AACCGCAAACATAAATG |
| | TCTGGGCGGCTGT | | AAGATCTGGTGTGC | | GTGCAAATATAAACTGTGG |
| 524 | GAATGCACCAAACT | 619 | AAAAGCGATGAAAACTGCTGCA | 723 | AACCGCCGCGATAAATG |
| | GCTGGGCGGCTGT | | AAAAATTTAAATGC | | GTGCAAATATAAACTGTGG |
| 525 | GAAGATTGCATTCC | 620 | AAAAGCACCAGCGATTGCTGC | 724 | AACAGCCGCGATAAATG |
| | GAAATGGAAAGGCTGT | | GAACATCTGAGCTGC | | GTGCAAAGTGCTGCTG |
| 526 | GAAGGCGAATGCG | 621 | AAAACCACCGCGGATTGCTGC | 725 | CAGCTGTGGTGCAAAAA |
| | GCGGCTTTTGGTGGAAATGT | | AAACATCTGGCGTGC | | ACGCCTG |
| 527 | GGCGCGTGCCGCT | 622 | AAAACCACCGCGGATTGCTGC | 726 | CAGCCGGCGATTAAATG |
| | GGTTTCTGGGCGGCTGT | | AAACATCTGGGCTGC | | GTGCATTTGGAGCCCG |
| 528 | GGCTGCGCGAACG | 623 | AAAACCACCAGCGATTGCTGCA | 727 | CAGAGCCGCATTGCGAA |
| | CGTATAAAAGCTGT | | AACATCTGGGCTGC | | CATGTGGCCGACCTTTTGCCTGGTG |
| 529 | GGCTGCGGCGGCC | 624 | AACGCGCTGAGCGGCCCGCGC | 728 | CAGAGCCGCATTGCGAA |
| | TGATGGCGGGCTGT | | TGCTGCAGCGGCCTGAAATGC | | CATGTGGCCGACCTTTTGCAGCCAG |
| 530 | GGCTGCGGCGGCC | 625 | AACGATGAAATGGTGTGCTGC | 729 | CGCAAAAAATGGCCGTA |
| | TGATGGATGGCTGT | | GAACATCTGGTGTGC | | TCATTGCGGCTGGGATGGCACCTTT |
| 531 | GGCTGCGGCACCA | 626 | AACGGCCCGCATACCTGCTGC | 730 | CGCAAAAAATGGCCGTA |
| | TGTGGAGCCCGTGT | | TGGGGCTATAACGGCTATAAAA | | TCATTGCGTGTGGGATTGGACCGTG |
| | | | AAGCGTGC | | |
| 532 | GGCTGCATTCCGAG | 627 | AACCCGGATAACGATAAATGCT | 731 | CGCCTGTGGTGCAAAAA |
| | CTTTGGCGAATGT | | GCGAAGGCCGCAAATGC | | AATTATT |
| 533 | GGCTGCAAAGGCTT | 628 | AACCCGAACGATGATAAATGCT | 732 | CGCCTGTGGTGCAAAAA |
| | TGGCGATAGCTGT | | GCCGCCCGAAACTGAAATGC | | AAAAATTGAAGAAGGC |
| 534 | GGCTGCAAACTGAC | 629 | AACCCGAGCAACGATAAATGCT | 733 | CGCCTGTGGTGCAAAAA |
| | CTTTTGGAAATGT | | GCCGCCCGAACCTGGTGTGC | | AAAAATTGAATGG |
| 535 | GGCTGCCTGGAATT | 630 | AACCCGAGCAACGATCAGTGC | 734 | CGCCTGTGGTGCAAAAA |
| | TTGGTGGAAATGT | | TGCAAAAGCGCGAACCTGGTG | | AAAACTGTGG |
| | | | TGC | | |
| 536 | GGCTGCCTGGGCG | 631 | AACCCGAGCAACGATCAGTGC | 735 | CGCCTGTGGTGCAAAAA |
| | ATAAATGT | | TGCAAAAGCAGCAAACTGGTGT | | ACGCCTG |
| | | | GC | | |
| 537 | GGCTGCAACCGCA | 632 | AACCCGAGCAACGATCAGTGC | 736 | CGCCTGTGGTGCAAACT |
| | AAAACAAAAAATGT | | TGCAAAAGCAGCAACCTGGTG | | GGATTGG |
| | | | TGC | | |
| 538 | GGCTGCCAGAAATT | 633 | AACAGCGATGCGGATTGCTGC | 737 | CGCCTGTGGTGCAAACG |
| | TTTTTGGACCTGT | | CGCTATGGCGAACGCTGC | | CATTATTAACATG |
| 539 | GGCGATTGCCTGC | 634 | AACAGCGATAAAGAATGCTGCA | 738 | CGCCTGTGGTGCAAATA |
| | CGCATCTGAAACTGTGT | | AAGGCCTGCGCTGC | | TAAACTG |
| 540 | GGCGATTGCCTGC | 635 | AACTATATGGATGATAAATGCT | 739 | CGCCGCGCGAAACCGAGCTG |
| | CGCATCTGAAACGCTGT | | GCCCGGGCTATAAATGC | | GTGCGGCTGGGATTTTACCTTT |
| 541 | GGCGGCTGCCTGC | 636 | CCGAAAAAAGCGCCGTGCTGC | 740 | CGCCGCGCGAAACCGAGCTGGT |
| | CGCATAACCGCTTTTGT | | GGCCGCCTGGAATGC | | GCGGCTGGGATTTTACCGTG |
| 542 | GGCGTGGATAAAGCGGGCTGCC | 637 | CCGTATCATGAAAGCTGCTGCA | 741 | CGCCGCACCCTGCCGACCTATTGC |
| | GCTATATGTTTGGCGGCTGT | | GCGGCAGCTGC | | GCGTGGGATCTGACCTTTCCG |
| 543 | GGCGTGGATAAAGAAGGCTGCCG | 638 | CCGTATAACGAACATTGCTGCA | 742 | CGCAGCGATGGCAAATA |
| | CAAACTGCTGGGCGGCTGT | | GCGGCAGCTGC | | TTGCGCGTGGGATGGCACCTTT |
| 544 | ATTGCGTGCGCGC | 639 | CCGTATAACGAAAACTGCTGCA | 743 | CGCAGCGATTGGAAATA |
| | CGCGCTTTAGCATTTGT | | GCAAAAGCTGC | | TTGCGCGTGGGATGGCACCTTTAGC |
| 545 | ATTGCGTGCGCGC | 640 | CCGTATAACGAAAACTGCTGCA | 744 | CGCAGCCGCGATCAGTG |
| | CGCGCTTTAGCCTGTGT | | GCCAGAGCTGC | | GTGCAAATATAAACTGTGG |
| 546 | ATTGCGTGCGCGC | 641 | CCGTATAACGAAAGCTGCTGCA | 745 | CGCGTGCGCGATCAGTG |
| | CGCGCGGCCTGCTGTGT | | GCGGCAGCTGC | | GTGCAAATATAAACTGTGG |
| 547 | AAATGCCTGCCGCC | 642 | CCGTATAGCAAATATTGCTGCA | 746 | AGCGATAAACATAAATG |
| | GGGCAAACCGTGT | | GCGGCAGCTGC | | GTGCAAATGGAAACTG |
| 548 | CTGTGCAGCCGCG | 643 | CAGCCGAACACCCAGCCGTGC | 747 | AGCCATAACAAATGCAC |
| | AAGGCGAATTT | | TGCAACAACGCGGAAGAAGAA | | C |
| | | | CAGACCATTAACTGC | | |
| 549 | CAGTGCGGCGAATT | 644 | CGCGAAAACAAAGATTGCTGCA | 748 | AGCAAACTGTTTAAACTG |
| | TATGTGGAAATGT | | GCAAAAAATGC | | TGCAACTTTAGCTTT |
| 550 | CGCTGCATTGAAGA | 645 | CGCCGCGATAGCGATTGCTGC | 749 | AGCAAAACCGGCTTTGT |
| | AGGCAAATGGTGT | | CCGCATCTGGGCTGC | | GAAAAACATTTGCAAATATGAAATG |
| 551 | AGCGCGGTGTGCATTCCGAG | 646 | AGCGCGGGCCAGACCTGCTGC | 750 | AGCAAAACCTGGGGCTG |
| | CGGCCAGCCGTGT | | AAACATCTGGTGTGC | | GTGCGCGGTGGAAGCGCCG |
| 552 | AGCTGCAAACTGAC | 647 | AGCGAAGATAGCGAATGCTGC | 751 | AGCCCGAAACATGGCTGGTGCGT |
| | CTTTTGGCGCTGT | | CCGCATCTGGGCTGC | | GTGGGATTGGACCTTTCGCAAA |
| 553 | AGCGAATGCCGCT | 648 | AGCAAAGATGCGGATTGCTGC | 752 | AGCCCGAAATGGGGCCT |
| | GGTTTATGGGCGGCTGT | | GCGCATCTGGAATGC | | GTGCAACTTTCCGATGCCG |
| 554 | AGCGAAAAAGATTG | 649 | AGCAAACATGAAGATTGCTGCG | 753 | AGCCCGCGCTGGGGCTGGTGCAT |
| | CATTAAACATCTGCAGCGCTGT | | CGCATCTGGCGTGC | | TTATAGCACCCGCGGCGGCCGC |
| 555 | AGCTTTTGCATTCC | 650 | AGCAAAACCGGCGATTGCTGC | 754 | AGCCCGACCTGGAAATGGTGCGTG |
| | GTTTAAACCGTGT | | AGCCATCTGAGCTGC | | CTGAAAAGCCCGGGCCGCCGC |
| 556 | AGCCCGACCTGCAT | 651 | AGCCAGGATGGCGATTGCTGC | 755 | AGCCCGACCTGGAAATG |
| | TCCGAGCGGCCAGCCGTGT | | AAACATCTGCAGTGC | | GTGCGTGTATGCGCGCCCG |
| 557 | AGCCCGACCTGCAT | 652 | AGCCAGACCAGCGATTGCTGC | 756 | AGCCAGCATCGCCTGTG |
| | TCCGACCGGCCAGCCGTGT | | CCGCATCTGGCGTGC | | CAGCGTGAAAGCG |
| 558 | AGCCCGACCTGCAT | 653 | AGCAGCGATAAACCGTGCTGC | 757 | AGCCGCAAAGATAAATG |
| | TCGCAGCGGCCAGCCGTGT | | AGCGGCTATTATTGC | | GTGCAAATATCAGATT |
| 559 | AGCCCGGTGTGCA | 654 | AGCAGCACCAGCGATTGCTGC | 758 | AGCCGCAAACATCGCTG |
| | CCCCGAGCGGCCAGCCGTGT | | AAACATCTGAGCTGC | | GTGCAAATATGAAATT |
| 560 | AGCAGCACCTGCAT | 655 | AGCACCGAAAAACCGTGCTGC | 759 | AGCCGCAAAACCCGCTG |
| | TCCGAGCGGCCAGCCGTGT | | GATAACTTTAGCTGC | | GTGCAAATATCAGATT |
| 561 | CGGCTGGCCGCTC | 656 | AGCACCCATGCGGATTGCTGC | 760 | AGCCGCCAGCTGTGCAA |
| | GGAATGCAGGTGCTGCTTGT | | GAAGGCTTTATTTGC | | ATATGTGATTGATTGG |
| 562 | AGCACCTGCACCCC | 657 | AGCGTGGATAGCGATTGCTGC | 761 | AGCCGCCGCGATCGCTG |
| | GACCGATCAGCCGTGT | | GCGCATCTGGGCTGC | | GTGCAAATATGATCTG |
| 563 | AGCGTGTGCATTCC | 658 | AGCGTGCATAGCGATTGCTGC | 762 | AGCCGCCGCGATCGCTG |
| | GAGCGGCCAGCCGTGT | | GCGCATCTGGGCTGC | | GTGCAAATATTATCTG |
| 564 | ACCTGCCGCTATCT | 659 | AGCGTGAACGATGATTGCTGC | 763 | AGCCGCCGCGGCACCAA |
| | GTTTGGCGGCTGT | | CCGCGCCTGGGCTGC | | CCCGGAAAAACGCTGCCGC |
| 565 | ACCTGCTATGATAT | 660 | ACCGATCGCCTGCCGTGCTGC | 764 | AGCCGCCGCCATGGCTG |
| | TGGCGAACTGTGT | | TTTGGCCTGGAATGC | | GTGCGTGTGGGATGGCACCTTTAGC |
| 566 | GTGTGCCGCGGCT | 661 | ACCATTGATGATGATTGCTGCC | 765 | AGCCGCACCTGGAAATG |
| | ATGGCCTGCCGTGT | | CGCATCTGGGCTGC | | GTGCGTGCTGGCGGGCCCGTGG |
| 567 | TATTGCCAGAAATG | 662 | ACCAAAGATGAAGATTGCTGCA | 766 | AGCAGCAAACATAAATG |
| | GCTGTGGACCTGT | | AACATCTGGCGTGC | | GTGCAAAGTGTATCTG |
| 568 | TATTGCCAGAAATG | 663 | ACCAAAGATAGCGAATGCTGC | 767 | AGCAGCCGCTGGAAATG |
| | GATGTGGACCTGT | | CCGCATCTGGGCTGC | | GTGCGTGCTGGCGAGCCCGTTT |
| 569 | TGCAAACAGGCGG | 664 | ACCCCGGAAAAAAACGATTGCT | 768 | AGCAGCCGCTGGAAATG |
| | ATGAACCGTGT | | GCCAGCGCCTGTATTGC | | GTGCGTGCTGCCGGCGCCGTGG |
| 570 | GCGTGCCGCAAAAA | 665 | ACCCCGGGCAAAAACGAATGC | 769 | ACCTTTAAAGAAAACGAA |
| | ATGGGAATATTGT | | TGCCCGAACCGCGTGTGC | | AACGGCAACACCGTGAAACGCTGCGAT |
| 571 | GATGATGATTGCGA | 666 | ACCCCGGGCAAAAACGAATGC | 770 | ACCTTTAAAACCAACGAA |
| | ACCGCCGGGCAACTTTTGT | | TGCCCGAACTATGCGTGC | | AACGGCAACACCGTGAAACGCTGCGAT |
| 572 | GTGAAACCGTGCC | 667 | ACCAGCGATAGCGATTGCTGC | 771 | ACCGGCCTGTGCATTCC |
| | GCAAAGAAGGCCAGCTGTGT | | CCGAACTGGGTGTGC | | GCCG |
| 573 | TGGTGCAAACAGAG | 668 | ACCACCAGCAGCGAATGCTGC | 772 | ACCCGCTTTAACGTGTG |
| | CGGCGAAATGTGT | | GCGCATCTGGGCTGC | | CGGCAAA |
| 574 | TGCCTGAGCGGCG | 669 | GTGAACCGCCATGGCGATTGC | 773 | ACCTGGCCGACCGAAAT |
| | GCGAAGTGTGT | | TGCGAAGGCCTGGAATGC | | TTGCATTGAT |
| 575 | GGCAAACCGTGCC | 670 | GTGCCGGGCAAAAACGAATGC | 774 | ACCTATAAAGCGAACGA |
| | ATGAAGAAGGCCAGCTGTGT | | TGCAGCGGCTATGCGTGC | | AAACGGCAACCAGGTGAAACGCTGCGAT |
| 576 | TGCATTCCGTTTCT | 671 | TATGGCGCGACCCAGAAAATTC | 775 | ACCTATAAAGAAAACGAA |
| | GCATCCGTGT | | CGTGCTGCGGCGTGTGC | | AACGGCAACACCGTGAAACGCTGCGAT |
| 577 | GCGTGCAGCAAAAA | 672 | TATAAACTGCGCAAATGCTGCG | 776 | ACCTATAAAGAAAACGAA |
| | ATGGGAATATTGT | | CGGGCTTTTATTGC | | AACGGCAACACCGTGCAGCGCTGCGAT |
| | | 673 | GATGTGTTTAGCCTGGATTGCT | 777 | ACCTATAAAACCAACGAA |
| | | | GCACCGGCATTTGC | | AACGGCAACAGCGTGCAGCGCTGCGAT |
| | | 674 | ATTGTGCCGATTATTGGCTTTA | 778 | GTGAAAACCAGCGGCTACTATTGTTGG |
| | | | TTTATTGCTGCCCGGGCCTGAT | | TGGTATAAAAAAACCCGCCGCAAAAGC |
| | | | TTGC | | |
| | | 675 | GGCATGATTAAAATTGGCCCGC | 779 | TGGAAACGCCGCCGCAGCTTTGAAG |
| | | | CGTGCTGCAGCGGCTGGTGC | | TGTGCGTGCCGAAAACCCCGAAAACC |
| | | 676 | GATCCGATTTTTCAGAACTGCT | 780 | CTGGGCGTGTGCATGTGG |
| | | | GCCGCGGCTGGAACTGC | | |
| | | 677 | AACGTGCTGGATCAGAACTGCT | 781 | TTTTTTGCGTGCGCG |
| | | | GCGATGGCTATTGC | | |
| | | 678 | GATTTTCTGTTTCCGAAATGCT | 782 | GTGCTGTTTTGCGTG |
| | | | GCAACTATTGC | | |
| | | 679 | GATCCGTTTCTGCAGAACTGCT | 783 | ATTGTGTTTGTGTGCACC |
| | | | GCCTGGGCTGGAACTGC | | |
| | | 680 | ACCTTTTTTTTTCCGGATTGCT | 784 | ATTCTGCTGTTTTGCAGC |
| | | | GCAACAGCATTTGC | | |
| | | 681 | ATTGTGCCGATTCTGGGCTTTG | 785 | GTGTTTGTGTGCATT |
| | | | TGTATTGCTGCCCGGGCCTGA | | |
| | | | TTTGC | | |
| | | | | 786 | GCGCAGTTTATTTGCCTG |
| | | | | 787 | GGCCCGTTTGTGTGCGTG |

In some embodiments, reference sequence element A has an amino acid sequence GCKWYLGDC (SEQ TD NO: 809). In some embodiments, reference sequence element A has an amino acid sequence SSTCIPSGQPC (SEQ ID NO: 255). In some embodiments, reference sequence element B has an amino acid sequence ADSDDCCETFHC (SEQ ID NO: 273). In some embodiments, reference sequence element C has an amino acid sequence KWVFFTSKFMCRRVWGKD (SEQ ID NO: 411).

In some embodiments, a provided polypeptide component has an amino acid sequence that is or comprises GCKWYLGDCADSDDCCETFHCKWVFFTSKFMCRRVWGKD (SEQ ID NO: 128), as is found in the Hv1 modulating agent labeled as “C5” in Table 2A.

In some embodiments, a provided polypeptide component has an amino acid sequence that is or comprises SSTCIPSGQPCADSDDCCETFHCKWVFFTSKFMCRRVWGKD (SEQ ID NO: 129), as is found in the Hv1 modulating agent labeled as “C6” in Table 2A.

In some embodiments, a polypeptide component has an amino acid sequence that includes one or more cysteine residues at positions corresponding to those at which a cysteine residue is found in a relevant wild-type toxin (e.g., wild-type voltage sensor toxin) sequence or reference sequence element (e.g., as depicted in FIG. 2). In some embodiments, a polypeptide component has an amino acid sequence that includes all cysteine residues at positions corresponding to those at which cysteine residues are found in a relevant wild-type toxin sequence or reference sequence element. In some embodiments, a polypeptide component has an amino acid sequence that shares the same approximate relative position of cysteines (e.g., number of residues between them) with a relevant wild-type toxin sequence or reference sequence element.

In some embodiments, a polypeptide component has an amino acid sequence that includes one or more sequence elements that are identical to or includes not more than 1, 2, 3, 4, or 5 sequence differences relative to a wild-type toxin sequence element or reference sequence element.

In some embodiments, such sequence difference(s) are or comprise one or more insertions, deletions, substitutions, rearrangements (e.g., inversions) or combinations thereof. In some embodiments, such sequence difference(s) do not include any insertions. In some embodiments, such sequence difference(s) do not include any deletions. In some embodiments, such sequence differences do not include any rearrangements (e.g., inversions). In some embodiments, such sequence difference(s) may include one or more random sequence alterations.

In some embodiments, a polypeptide component has an amino acid sequence that includes one or more sequence elements that shares one or more cysteines with a sequence set forth in Table 1, Table 2, and/or Table 3. In some embodiments, such a sequence element has an amino acid sequence that shares all cysteines with a sequence set forth in Table 1, Table 2, and/or Table 3. In some embodiments, a sequence element shares the same approximate relative position of cysteines (e.g., number of residues between them) with a sequence set forth in Table 1, Table 2, and/or Table 3.

In some embodiments, a reference sequence element has an amino acid sequence of an element found in a wild-type voltage toxin sequence that differs at residues that undergo posttranslational modifications.

In some embodiments, a polypeptide component of an Hv1 modulating agent may include one or more pendant groups or other modifications, e.g., modifying or attached to one or more amino acid residues (e.g., to one or more amino acid side chains), at the polypeptide component's N-terminus, at the polypeptide component's C-terminus, or any combination thereof. In some embodiments, such pendant groups or modifications may be selected from the group consisting of acetylation, amidation, glycosylation, lipidation, methylation, pegylation, phosphorylation, etc., and combinations thereof.

In some embodiments, exemplary Hv1 modulating agents comprise a polypeptide component whose amino acid sequence further comprises one or more tag elements (e.g., a detectable tag, a localizing tag, etc).

In some embodiments, an Hv1 modulating agent may be a dimer or multimer of relevant entities (e.g., of a polypeptide component as described herein); in some embodiments, an Hv1 modulating agent may be or comprise heterodimer or heteromultimer. In some embodiments, an Hv1 modulating agent may be or comprise a homodimer or homomultimer. Exemplary Hv1 modulating agent dimers are presented in Table 4.

TABLE 4A
Dimer Sequences (with linkers)

| SEQ | | |
|---|---|---|
| ID | | |
| NO: | Name | Sequence |
| 788 | HaTx-C6 | ECRYLEGGCKTTSDCCKHLGCKFR |
| | | DKYCAWDETFSGNGNGNGSSTCIP |
| | | SGQPCADSDDCCETFHCKWVFFTS |
| | | KFMCRRVWGKD |
| 789 | C6-C6 | SSTCIPSGQPCADSDDCCETFHCK |
| | with | WVFFTSKFMCRRVWGKDDSSPYVP |
| | DkTx | VTTSSTCIPSGQPCADSDDCCETF |
| | linker | HCKWVFFTSKFMCRRVWGKD |
| 790 | C6-C6 | SSICIPSGQPCADSDDCCETFHCK |
| | with | WVFFTSKFMCRRVWGKDDSSGNGN |
| | flexible | GNGSSTCIPSWPCADSDDCCETFE |
| | linker | ICKWVFFTSKFMCRRVWGKD |
| 791 | C6-C6 | SSTCIPSGQPCADSDDCCETFHCK |
| | with | WVFFTSKFMCRRVWGKDDSSGGNG |
| | long | NGNGNGNGNGNGAAAGGNGNGNGN |
| | flexible | GNGNGNGSSTCIPSGQPCADSDDC |
| | linker | CETFHCKWVFFTSKFMCRRVWGKD |

TABLE 4B
Monomer C6 + signal AND dimer + signal

| SEQ | | |
|---|---|---|
| ID | | |
| NO: | Name | Sequence |
| 792 | C6 with signal | MSALLILALVGAAVA |
| | peptide | SSTCIPSGQPCADSD |
| | (MSALLILALVGAAVA) | DCCETFHCKWVFFTS |
| | | KFMCRRVWGKD |
| 793 | HaTx-C6 | MSALLILALVGAAVA |
| | | ECRYLFGGCKTTSDC |
| | | CKHLGCKFRDKYCAW |
| | | DFTFSGNGNGNGSST |
| | | CIPSGQPCADSDDCC |
| | | ETFHCKWVFFTSKFM |
| | | CRRVWGKD |
| 794 | C6-C6 with DkTx | MSALLILALVGAAVA |
| | linker with signal | SSTCIPSGQPCADSD |
| | peptide | DCCETFHCKWVFFTS |
| | | KFMCRRVWGKDDSSP |
| | | YVPVTTSSTCIPSGQ |
| | | PCADSDDCCETFHCK |
| | | WVFFTSKFMCRRVWG |
| | | KD |
| 795 | C6-C6 with | MSALLILALVGAAVA |
| | flexible linker | SSTCIPSGQPCADSD |
| | with signal | DCCETFHCKWVFFTS |
| | peptide | KFMCRRVWGKDDSSG |
| | | NGNGNGSSTCIPSGQ |
| | | PCADSDDCCETFHCK |
| | | WVFFTSKFMCRRVWG |
| | | KD |
| 796 | C6-C6 with long | MSALLILALVGAAVA |
| | flexible linker | SSTCIPSGQPCADSD |
| | with signal | DCCETFHCKWVFFTS |
| | peptide | KFMCRRVWGKDDSSG |
| | | GNGNGNGNGNGNGNG |
| | | AAAGGNGNGNGNGNG |
| | | NGNGSSTCIPSGQPC |
| | | ADSDDCCETFHCKWV |
| | | FFTSKFMCRRVWGKD |

TABLE 4C
Monomer + Mye tag, dimer + Myc tag,
either/both plus signal and tag

| SEQ ID | | |
|---|---|---|
| NO: | Name | Sequence |
| 797 | C6 with signal | MSALLILALVGAAVA |
| | peptide and | SSTCIPSGQPCADSD |
| | Myc tag | DCCETFHCKWVFFTS |
| | | KFMCRRV |
| | | WGKDGEQKLISEEDL |
| 798 | HaTx-C6 with | MSALLILALVGAAVA |
| | signal peptide | ECRYLFGGCKTTSDC |
| | and Myc tag | CKHLGCKFRDKYCAW |
| | | DFTFSGN |
| | | GNGNGSSTCIPSGQP |
| | | CADSDDCCETFHCKW |
| | | VFFTSKFMCRRVWGK |
| | | DGEQKLI |
| | | SEEDL |
| 799 | C6-C6 with | MSALLILALVGAAVA |
| | DkTx linker | SSTCIPSGQPCADSD |
| | with signal | DCCETFHCKWVFFTS |
| | peptide and | KFMCRRV |
| | Myc tag | WGKDDSSPYVPVTTS |
| | | STCIPSGQPCADSDD |
| | | CCETFHCKWVFFTSK |
| | | FMCRRVW |
| | | GKDGEQKLISEEDL |
| 800 | C6-C6 with | MSALLILALVGAAVA |
| | flexible | SSTCIPSGQPCADSD |
| | linker with | DCCETFHCKWVFFTS |
| | signal peptide | KFMCRRV |
| | and Myc tag | WGKDDSSGNGNGNGS |
| | | STCIPSGQPCADSDD |
| | | CCETFHCKWVFFTSK |
| | | FMCRRVW |
| | | GKDGEQKLISEEDL |
| 801 | C6-C6 with | MSALLILALVGAAVA |
| | long flexible | SSTCIPSGQPCADSD |
| | | DCCETFHCKWVFFTS |
| | | KFMCRRV |
| | | WGKDDSSGGNGNGNG |
| | | NGNGNGNGAAAGGNG |
| | | NGNGNGNGNGNGSST |
| | | CIPSGQPCADSDDCC |
| | | ETFHCKWVFFTSKFM |
| | | CRRVWGKDGEQKLIS |
| | | EEDL |

TABLE 4D
Monomer + GPI targeting, dimer + GPI targeting,
either/both plus signal,
tag and linker

| SEQ | | |
|---|---|---|
| ID | | |
| NO: | Name | Sequence |
| 802 | C6 with signal | MSALLILALVGAAVASSTCIPSGQ |
| | peptide and Myc | PCADSDDCCETFHCKWVFFTSKFM |
| | tag and GPI | CRRVWGKDGEQKLISEEDLGALCN |
| | anchor | GAGFATPVTLALVPALLATFWSLL |
| 803 | HaTx-C6 with | MSALLILALVGAAVAECRYLFGGC |
| | signal peptide | KTTSDCCKHLGCKFRDKYCAWDFT |
| | and Myc tag and | FSGNGNGNGSSTCIPSGQPCADSD |
| | GPI anchor | DCCETFHCKWVFFTSKFMCRRVWG |
| | | KDGEQKLISEEDLGALCNGAGFAT |
| | | PVTLALVPALLATFWSLL |
| 804 | C6-C6 with DkTx | MSALLILALVGAAVASSTCIPSGQ |
| | linker with | PCADSDDCCETFHCKWVFFTSKFM |
| | signal peptide | CRRVWGKDDSSPYVPVTTSSTCIP |
| | and Myc tag and | SGQPCADSDDCCETFHCKWVFFTS |
| | GPI anchor | KFMCRRVWGKDGEQKLISEEDLGA |
| | | LCNGAGFATPVTLALVPALLATFW |
| | | SLL |
| 805 | C6-C6 with | MSALLILALVGAAVASSTCIPSGQ |
| | flexible linker | PCADSDDCCETFHCKWVFFTSKFM |
| | with signal | CRRVWGKDDSSGNGNGNGSSTCIP |
| | peptide and Myc | SGQPCADSDDCCETFHCKWVFFTS |
| | tag and GPI | KFMCRRVWGKDGEQKLISEEDLGA |
| | anchor | LCNGAGFATPVTLALVPALLATFW |
| | | SLL |
| 806 | C6-C6 with long | MSALLILALVGAAVASSTCIPSGQ |
| | flexible linker | PCADSDDCCETFHCKWVFFTSKFM |
| | with signal | CRRVWGKDDSSGGNGNGNGNGNGN |
| | peptide and Myc | GNGAAAGGNGNGNGNGNGNGNGSS |
| | tag and GPI | TCIPSGQPCADSDDCCETFHCKWV |
| | anchor | FFTSKFMCRRVWGKDGEQKLISEE |
| | | DLGALCNGAGFATPVTLALVPALL |
| | | ATFWSLL |

Optionally, monomer components of a dimer or multimer agent may be or become covalently linked to one another. In some embodiments, such components may be or become covalently linked to one another via one or more disulfide bonds; in some embodiments, such components may be or become covalently linked to one another via one or more peptide bonds; in some embodiments, such components may be or become covalently linked to one another via a bond other than a peptide bond. In some embodiments, such components may be or become covalently linked to one another via a linker (e.g., via a polypeptide linker). Those skilled in the art will appreciate that a linker may be comprised of any of a variety of chemical entities. In those embodiments in which a linker comprises one or a plurality of amino acids, it may be of any desired length. In some embodiments, a linker has a size (e.g., a length) that is smaller than that of one or more of the monomer components.

In some embodiments, a polypeptide component of an Hv1 modulating agent may be connected directly or via a linker sequence to a signal peptide and/or a coat protein of a phage (for phage display methods) and/or to any other domain that may alter one or more of Hv1 modulating agent expression, binding, or function.

In some embodiments, an Hv1 modulating agent may have structural modification(s). For example, an Hv1 modulating agent may be or comprise a cyclic structure, and/or may comprise a cyclic portion. For example, a polypeptide component of an Hv1 modulating agent may be cyclized such that its N-terminus is not part of the cyclic structure. In some embodiments, an Hv1 modulating agent is not cyclic and/or does not comprise any cyclic portion. In some embodiments, an Hv1 modulating agent is linear (e.g., one or more, or all, polypeptide components of an Hv1 modulating agent is/are linear polypeptide(s)). In some embodiments, an Hv1 modulating agent may be or comprise a stapled polypeptide.

In some embodiments, a polypeptide component of an Hv1 modulating agent is incorporated into a framework or scaffold structure. For example, such a polypeptide component can be incorporated into an antibody framework. Alternatively or additionally, a polypeptide component may be incorporated into a beta-sheet framework. In some embodiments, an Hv1 modulating agent may be or comprise an antibody agent or fragment or component thereof (e.g., an antigen-binding fragment or component, such as a polypeptide including sufficient CDR sequences to bind antigen comparably to an antibody). In some embodiments, an Hv1 modulating agent may be or comprise a polypeptide component that includes one or more of an immunoglobulin domain or fragment thereof. In some embodiments, an Hv1 modulating agent may be or comprise a polypeptide component that includes a domain of an immunoglobulin heavy chain. Strategies for preparing such antibody fusions are known in the art (U.S. Ser. No. 14/152,441).

Alternatively or additionally, in some embodiments, an Hv1 modulating agent may be or comprise a nucleic acid, for example that may encode a polypeptide having Hv1 modulating agent activity and/or structure, as described herein. Exemplary nucleic acid sequences for Hv1 modulating agents are illustrated in Table 2C.

### Production of Hv1 Modulating Agents

Hv1 modulating agents can be produced by many methods. In some embodiments, an Hv1 modulating agent is produced by recombinant expression in a cell. In some embodiments, an Hv1 modulating agent is produced by peptide synthesis. In some embodiments, an Hv1 modulating agent is produced by in vitro translation.

Exemplary methods of designing and producing Hv1 modulating agents are presented in Example 1.

In some embodiments, an Hv1 modulating agent is presented on a replicable genetic package, e.g., in the form of a phage, yeast, ribosome, or nucleic acid-protein fusion.

In some embodiments, an Hv1 modulating agent is provided and/or utilized in the context of an expression or display system.

In some embodiments, Hv1 modulating agents are first synthesized as nucleic acids that encode polypeptide elements (e.g. elements A, B, and C in Table 3) and then annealed to produce nucleic acid sequences encoding polypeptide components (e.g. as in Table 2).

In some embodiments, a nucleic acid sequence encoding an Hv1 modulating agent may be inserted into a phagemid or phage vector, in-frame, to form a leader-linker-Hv1 modulating agent-linker-coat protein construct (Clackson and Lowman, Phage display. Oxford University Press, 2004; Barbas et al., Phage display. A laboratory manual. Cold Spring Harbor Laboratory Press, 2001). Exemplary upstream leader and downstream amino acid sequences are MAAE and GSASSA, respectively. An exemplary phage vector is pAS62. An exemplary coat protein is protein III or its truncated version. Phages can be grown, prepared, titered and stored (Clackson and Lowman, Phage display. Oxford University Press, 2004; Barbas et al., Phage display. A laboratory manual. Cold Spring Harbor Laboratory Press, 2001).

In some embodiments, Hv1 modulating agents can be inserted into vectors for expression and/or library selection. In some embodiments, a library is presented in a polypeptide array. In some embodiments, a library is presented on a replicable genetic package, e.g., in the form of a phage display, yeast display, ribosome, or nucleic acid-protein fusion library. See, e.g., U.S. Pat. No. 5,223,409; Garrard et al. (1991) Bio/Technology 9:1373-1377; WO 03/029456; and U.S. Pat. No. 6,207,446. Binding members of such libraries can be obtained by selection and screened in a high throughput format. See, e.g., U.S. 2003-0129659.

Hv1 modulating agent libraries for phage display can be generated by standard methods (Clackson and Lowman, Phage display. Oxford University Press, 2004; Barbas et al., Phage display. A laboratory manual. Cold Spring Harbor Laboratory Press, 2001). For example, Hv1 modulating agent libraries for phage display with a combinatorial arrangement of sequence elements are generated by designing overlapping or non-overlapping oligonucleotides corresponding to each individual element. These oligonucleotides are phosphorylated, annealed, mixed in a desired combination and concentration and ligated into a phagemid vector with or without linker sequences to create a library by standard methods (Sambrook et al., Molecular Cloning: A Laboratory Manual. Vols 1-3. Cold Spring Harbor Laboratory Press, 1989). Combinatorial arrangement of sequence elements to yield phage particles expressing a library of Hv1 modulating agents is demonstrated in Example 1A.

Identification and/or Characterization of Hv1 Modulating Agents

Identification and/or characterization of Hv1 modulating agents can include determining effects of candidate agents on Hv1, including Hv1 that is naturally or recombinantly expressed. In some embodiments, Hv1 is expressed in cells. In some embodiments, Hv1 is immobilized (e.g., immobilized on a solid support, an artificial membrane, or a plasma membrane of a cell). In some embodiments, Hv1 is purified.

Identification and/or characterization of Hv1 modulating agents can include the use of libraries of candidate agents.

In some embodiments, an Hv1 modulating agent is identified from a candidate library incorporated into a phage display system. In phage display, candidate Hv1 modulating agents are functionally displayed on the surface of phage and nucleic acid sequences encoding candidate Hv1 modulating agents are enclosed inside phage particles. Functional display permits selection of Hv1 modulating agents that interact with a target or targets (e.g. Hv1 channels). Selection of Hv1 modulating agents from the library can be based on the Hv1 modulating agent type (e.g., toxin type) and/or target biochemistry, pharmacology, immunology and/or other physicochemical or biological property.

A phage library can be transfected into Escherichia coli (E. coli) or other suitable bacterial species, propagated, and the phages purified. At this stage, Hv1 modulating agents or candidate agents can be functionally expressed on the surface of the phage and physically linked to their respective genes inside of the phage particle. A library is brought into contact with a target, such as Hv1 channels. For example, a phage library can be brought into contact with Hv1 channels that are immobilized on magnetic beads, as described in Example 2. After incubation with the target, those phages that express candidate Hv1 modulating agents with no or weak recognition for the target are washed away. The remaining Hv1 modulating agents that interact with the target are dissociated and can be (i) genotyped to establish the Hv1 modulating agent identity, or (ii) processed for one or more rounds of panning, or (iii) otherwise quantified and/or identified (e.g., ELISA, microbiological titering, functional testing).

Panning may be performed by the binding of candidate modulating agents to Hv1, followed by washes and modulating agent recovery. Panning may be repeated until the desired enrichment is achieved. In addition, libraries can be pre-depleted on surfaces or cells that contain no Hv1 or on an Hv1 where the putative modulating agent binding domain may be directly or indirectly altered. Additionally, any and all conditions of panning may be varied, altered or changed to achieve optimal results, such as the isolation of a specific Hv1 modulating agent. Panning variations include, but are not limited to, the presence of competing polypeptide(s), presence of excess target(s), length and temperature of binding, pre-absorption of the library on one or more different receptor(s) or cells or surfaces, composition of binding solution (e.g., ionic strength), stringency of washing, and recovery procedures. Phages recovered from panning may be processed for further rounds of panning, functional analysis, and/or sequencing/genotyping to deduce the resulting Hv1 modulating agent's amino acid sequence or biological properties (Clackson and Lowman, Phage display. Oxford University Press, 2004; Barbas et al., Phage display. A laboratory manual. Cold Spring Harbor Laboratory Press, 2001).

Following recovery after panning, Hv1 modulating agents of interest may be produced in native form by standard methods of peptide/protein synthesis/production (Sambrook et al., Molecular Cloning: A Laboratory Manual. Vols 1-3. Cold Spring Harbor Laboratory Press, 1989; Albericio, Solid-Phase Synthesis: A Practical Guide. CRC, 2000; Howl, Peptide Synthesis and Applications. Humana Press, 2005).

In some embodiments, Hv1 modulating agents are tested for activity toward recombinant or functional Hv1. Samples that include functional channels (e.g., cells or artificial membranes) can be treated with an Hv1 modulating agent and compared to control samples (e.g., samples without the Hv1 modulating agent), to examine the extent of modulation. In some embodiments, Hv1 may be naturally expressed.

In some embodiments, cells may be stably or transiently transfected with functional Hv1. For example, HEK-293T (mammalian human embryonic kidney) cells may be transfected with Hv1 (e.g. human Hv1 or human Hv1 tagged with a fluorescent protein) for transient expression. In one example, HEK-293T cells transiently expressing hHv1 tagged with teal fluorescent protein are used in patch clamp electrophysiology to determine effects of the Hv1 modulating agents C5 or C6 on proton currents.

Changes in proton flux may be assessed by determining changes in polarization (i.e., electrical potential) of a cell or membrane expressing Hv1. In some embodiments, a change in cellular polarization is measured by measuring changes in current (thereby measuring changes in polarization) with voltage-clamp and patch-clamp techniques, e.g., the “cell-attached” mode, the “inside-out” mode, and the “whole cell” mode (see, e.g., Ackerman et al., New Engl. J. Med. 336:1575-1595, 1997). Whole cell currents can be determined using standard methodology (see, e.g., Hamil et al., PFlugers. Archiv. 391:85, 1981). Other assays include radiolabeled rubidium flux assays and fluorescence assays using voltage sensitive dyes (see, e.g., Vestergarrd-Bogind et al., J. Membrane Biol. 88:67-75, 1988; Daniel et al., J. Pharmacol. Meth. 25:185-193, 1991; Holevinsky et al., J. Membrane Biology 137:59-70, 1994). In some embodiments, candidate Hv1 modulating agents are present in the range from 1 μM to 100 mM. Other methods for assessing Hv1 modulating agent effects on proton flux are described in the Examples herein.

Hv1 modulating agents can also be identified or characterized by evaluating processes at the cellular, tissue and/or organism level. For example, Hv1 modulating agents can be evaluated for effects downstream of Hv1 activity or signaling. Various effects of Hv1 modulating agents that may be determined using intact cells or animals include transcriptional changes, changes in cell metabolism, and changes in intracellular second messengers.

In some embodiments, Hv1 modulating agents can be evaluated for effects on human sperm. For example, Hv1 modulating agents can be evaluated for effects on sperm capacitation-related processes, including changes in sperm motility, decrease of cholesterol in the membranes, increase of tyrosine phosphorylation in several proteins, or maturation of the sperm response to progesterone. Capacitation of spermatozoa occurs along with an increase in the amplitude of voltage-gated proton current. Known Hv1 inhibitor, Zn²⁺, reduces H⁺ current in sperm cells. Accordingly, an Hv1 modulating agent can be evaluated for suppression of such sperm capacitation-related processes. Alternatively, an Hv1 modulating agent can be evaluated for enhancing sperm capacitation-related processes. Alternatively or additionally, an Hv1 modulating agent can be evaluated for non-capacitation-related processes that affect sperm activation, mobility, and/or fertilization.

In some embodiments, Hv1 modulating agents can be evaluated for effects on cells that function in the immune system. For example, Hv1 is expressed in white blood cells (WBCs). Hv1 in WBCs has been shown to compensate charge buildup on the cell membrane during production of ROS. Hv1 knockout or inhibition impairs ROS production in these cells. Accordingly, an Hv1 modulating agent can be evaluated for suppression of ROS production in WBCs.

Hv1 modulating agents can be selected for their potency and selectivity of modulation of Hv1. For example, an Hv1 modulating agent that demonstrates low ICso value for Hv1, and a higher ICso value for other ion channels within the test panel, is considered to be selective toward Hv1.

### Compositions

The present disclosure also features compositions that include and/or deliver Hv1 modulating agents.

In some embodiments, a composition is a pharmaceutically acceptable composition that includes and/or delivers an Hv1 modulating agent described herein. For example, in some embodiments, a provided composition includes an Hv1 modulating agent polypeptide component. Alternatively or additionally, in some embodiments, a provided composition includes a nucleic acid that encodes an Hv1 modulating agent polypeptide component, a cell that expresses (or is adapted to express) an Hv1 modulating agent polypeptide component, etc. In some embodiments Hv1 modulating agents having any of the modifications of the present disclosure are included in pharmaceutical compositions.

General considerations in the formulation and manufacture of pharmaceutical agents may be found, for example, in Remington's Pharmaceutical Sciences, 19^{th} ed., Mack Publishing Co., Easton, PA, 1995.

Pharmaceutical compositions may be in a variety of forms. These include, for example, liquid, semi-solid and solid dosage forms, such as liquid solutions (e.g., injectable and infusible solutions), dispersions or suspensions, microemulsions, liposomes and suppositories. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin. The preferred form of pharmaceutical composition depends on the intended mode of administration and therapeutic application. Typical compositions are in the form of injectable or infusible solutions, such as compositions similar to those used for administration of antibodies to humans.

The pharmaceutical composition can include a pharmaceutically acceptable carrier. For example, pharmaceutical compositions can include a therapeutic agent in addition to one or more inactive agents such as a sterile, biocompatible carrier.

Exemplary carriers include any and all solvents, dispersion media, coatings, antibacterial and antifungal agents, isotonic and absorption delaying agents, and the like that are physiologically compatible. For example, carriers may include sterile water, saline, buffered saline, or dextrose solution. Alternatively or additionally, the composition can contain any of a variety of additives, such as stabilizers, buffers, excipients (e.g., sugars, amino acids, etc.), or preservatives. Preferably, the carrier is suitable for oral, intravenous, intramuscular, subcutaneous, parenteral, spinal or epidermal administration (e.g., by injection or infusion).

The pharmaceutical composition can include a pharmaceutically acceptable salt, e.g., a salt that retains the desired biological activity of the Hv1 modulating agent and does not impart any undesired toxicological effects (see e.g., Berge, S. M., et al., J. Pharm. Sci. 66:1-19, 1977).

Depending on the route of administration, the Hv1 modulating agent may be coated in a material to protect the compound from the action of acids and other natural conditions that may inactivate the compound.

In certain embodiments, a pharmaceutical composition can include a therapeutic agent that is encapsulated, trapped, or bound within a lipid vesicle, a bioavailable and/or biocompatible and/or biodegradable matrix, or other microparticles.

In certain embodiments, an Hv1 modulating agent is prepared with a carrier that protects against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and poly lactic acid. Many methods for the preparation of such formulations are patented or generally known. See, e.g., Sustained and Controlled Release Drug Delivery Systems, J. R. Robinson, ed., Marcel Dekker, Inc., New York, 1978. Pharmaceutical formulation is a well-established art, and is further described in Gennaro (ed.), Remington: The Science and Practice of Pharmacy, 20.sup.th ed., Lippincott, Williams & Wilkins, 2000 (ISBN: 0683306472); Ansel et al., Pharmaceutical Dosage Forms and Drug Delivery Systems, 7.sup.th Ed., Lippincott Williams & Wilkins Publishers, 1999 (ISBN: 0683305727); and Kibbe (ed.), Handbook of Pharmaceutical Excipients American Pharmaceutical Association, 3rd ed., 2000 (ISBN: 091733096X).

In some embodiments, a provided pharmaceutical composition will include an Hv1 modulating agent that is not aggregated. For example, in some embodiments, less than 1%, 2%, 5%, 10%, 20%, or 30%, by dry weight or number, of Hv1 modulating agent is present in an aggregate.

In some embodiments, a provided pharmaceutical composition will include an Hv1 modulating agent that is not denatured. For example, less than 1%, 2%, 5%, 10%, 20%, or 30%, by dry weight or number, of Hv1 modulating agents administered is denatured.

In some embodiments, a provided pharmaceutical composition will include an Hv1 modulating agent that is not inactive. For example, less than 1%, 2%, 5%, 10%, 20%, or 30%, by dry weight or number, of Hv1 modulating agents administered is inactive.

In some embodiments, pharmaceutical compositions are formulated to reduce immunogenicity of provided Hv1 modulating agents. For example, in some embodiments, a provided Hv1 modulating agent is associated with (e.g., bound to) an agent, such as polyethylene glycol and/or carboxymethyl cellulose, that masks its immunogenicity. In some embodiments, a provided binding agent has additional glycosylating that reduces immunogenicity.

### Kits

Also provided by the present disclosure are kits that include an Hv1 modulating agent described herein and instructions for use, e.g., treatment, prophylactic, or diagnostic use.

In addition to the Hv1 modulating agent, the composition of the kit can include other ingredients, such as a solvent or buffer, a stabilizer or a preservative, and/or a second agent for treating a condition or disorder described herein. Alternatively, other ingredients can be included in the kit, but in different compositions or containers than the Hv1 modulating agent. In such embodiments, the kit can include instructions for admixing the Hv1 modulating agent and the other ingredients, or for using the Hv1 modulating agent together with the other ingredients.

Alternatively or additionally, contents of kits may include, but are not limited to, expression plasmids containing nucleotides (or characteristic or biologically active portions) encoding Hv1 modulating agents of interest (or characteristic or biologically active portions). Alternatively or additionally, kits may contain expression plasmids that express Hv1 modulating agents of interest (or characteristic or biologically active portions). Alternatively or additionally, kits may contain isolated and stored Hv1 modulating agents.

In certain embodiments, kits for use in accordance with the present invention may include, a reference sample, instructions for processing samples, performing tests on samples, instructions for interpreting the results, buffers and/or other reagents necessary for performing tests. In certain embodiments the kit can comprise a panel of antibodies.

The present invention provides kits for administration of pharmaceutical compositions. For example, in some embodiments, the invention provides a kit comprising at least one dose of an Hv1 modulating agent. In some embodiments, the invention provides a kit comprising an initial unit dose and one or more subsequent unit doses of an Hv1 modulating agent. In some such embodiments, the initial unit dose is greater than the subsequent unit doses or wherein all of the doses are equal.

### Methods of Administration

Pharmaceutical compositions may be administered in any dose appropriate to achieve a desired outcome. In some embodiments, the desired outcome is reduction in intensity, severity, and/or frequency, and/or delay of onset of one or more symptoms of an Hv1 associated disease or condition.

A therapeutically effective amount of an Hv1 modulating agent composition can be administered, typically an amount which is effective, upon single or multiple dose administration to a subject, in treating a subject, e.g., curing, alleviating, relieving or improving at least one symptom of a disease or condition in a subject to a degree beyond that expected in the absence of such treatment. A therapeutically effective amount of the composition may vary according to factors such as the disease state, age, sex, and weight of the individual, and the ability of the compound to elicit a desired response in the individual. A therapeutically effective amount is also one in which any toxic or detrimental effects of the composition is outweighed by the therapeutically beneficial effects. A therapeutically effective dosage preferably modulates a measurable parameter, favorably, relative to untreated subjects. The ability of an Hv1 modulating agent to inhibit a measurable parameter can be evaluated in an animal model system predictive of efficacy in a human disorder.

In some embodiments, pharmaceutical compositions are administered in multiple doses. In some embodiments, pharmaceutical compositions are administered in multiple doses/day. In some embodiments, pharmaceutical compositions are administered according to a continuous dosing regimen, such that the subject does not undergo periods of less than therapeutic dosing interposed between periods of therapeutic dosing. In some embodiments, pharmaceutical compositions are administered according to an intermittent dosing regimen, such that the subject undergoes at least one period of less than therapeutic dosing interposed between two periods of therapeutic dosing.

Dosage regimens can be adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of ligand calculated to produce the desired therapeutic effect in association with the required pharmaceutical carrier.

An exemplary, non-limiting range for a therapeutically or prophylactically effective amount of an Hv1 modulating agent described herein is 0.1-20 mg/Kg, more preferably 1-10 mg/Kg. In some embodiments, an agent can be administered by parenteral (e.g., intravenous or subcutaneous) infusion at a rate of less than 20, 10, 5, or 1 mg/min to reach a dose of about 1 to 50 mg/m² or about 5 to 20 mg/m². It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions (e.g., the supervising physician), and that dosage ranges set forth herein are only exemplary.

Pharmaceutical compositions of the present invention can be administered by a variety of routes, including oral, intravenous, intramuscular, intra-arterial, subcutaneous, intraventricular, transdermal, interdermal, rectal, intravaginal, intraperitoneal, topical (as by powders, ointments, creams, or drops), mucosal, nasal, buccal, enteral, sublingual; by intratracheal instillation, bronchial instillation, and/or inhalation; and/or as an oral spray, nasal spray, and/or aerosol. For example, for therapeutic applications, an Hv1 modulating agent composition can be administered by intravenous infusion at a rate of less than 30, 20, 10, 5, or 1 mg/min to reach a dose of about 1 to 100 mg/m² or 7 to 25 mg/m². Alternatively, the dose could be 100 μg/Kg, 500 μg/Kg, 1 mg/Kg, 5 mg/Kg, 10 mg/Kg, or 50 mg/Kg. The route and/or mode of administration will vary depending upon the desired results. In general the most appropriate route of administration will depend upon a variety of factors including the nature of the agent (e.g., its stability in the environment of the gastrointestinal tract), the condition of the patient (e.g., whether the patient is able to tolerate oral administration), etc.

A common mode of administration is parenteral (e.g., intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion). In one embodiment, the Hv1 modulating agent composition is administered by intravenous infusion or injection. In another embodiment, the Hv1 modulating agent composition is administered by intramuscular or subcutaneous injection. In another embodiment, the Hv1 modulating agent composition is administered orally. In some embodiments, the Hv1 modulating agent composition is administered topically. In some embodiments, the Hv1 modulating agent composition is administered transdermally. Pharmaceutical compositions typically must be sterile and stable under the conditions of manufacture and storage.

Hv1 modulating agents or pharmaceutical compositions of the present disclosure may be administered either alone or in combination with one or more other agents. In some embodiments, Hv1 modulating agents or pharmaceutical compositions of the present disclosure may be administered with one or more other Hv1 modulating agents. In some embodiments, Hv1 modulating agents or pharmaceutical compositions of the present disclosure may be administered with one or more other pharmaceutical agent including, but not limited to, small molecules, vaccines and/or antibodies. In some embodiments, Hv1 modulating agents or pharmaceutical compositions may be administered in combination with an adjuvant.

Combinations of agents may be administered at the same time or formulated for delivery together. Alternatively, each agent may be administered at a dose and on a time schedule determined for that agent. Additionally, the invention encompasses the delivery of the pharmaceutical compositions in combination with agents that may improve their bioavailability, reduce or modify their metabolism, inhibit their excretion, or modify their distribution within the body. Although the pharmaceutical compositions of the present invention can be used for treatment of any subject (e.g., any animal) in need thereof, they are most preferably used in the treatment of humans.

### Uses

As described herein, Hv1 channels have been reported to play a role in a variety of biological processes, and to impact various diseases, disorders, and conditions.

The present disclosure encompasses treatment of Hv1 associated diseases or conditions. Hv1 modulating agents and/or Hv1 modulating agent compositions described herein can be administered, alone or in combination with, another agent to a subject, e.g., a patient, e.g., a patient who has a disorder (e.g., an Hv1-associated disease or condition, e.g. immune deficiency), a symptom of a disorder or a predisposition toward a disorder, with the purpose to cure, heal, alleviate, relieve, alter, remedy, ameliorate, improve or affect the disorder, the symptoms of the disorder or the predisposition toward the disorder. The treatment may also delay onset, e.g., prevent onset, or prevent deterioration of a condition.

In some embodiments, Hv1 modulating agent pharmaceutical compositions are administered to a subject suffering from or susceptible to an Hv1 associated disease or condition. In some embodiments, a subject is considered to be suffering from an Hv1 associated disease or condition if the subject is displaying one or more symptoms commonly associated with said disease or condition. Hv1 modulating agents or Hv1 modulating agent compositions may be administered prior to or after development of one or more such symptoms.

For example Hv1 modulating agents may be used to ameliorate inflammation, allergies, autoimmunity, cancer, asthma, brain damage from ischemic stroke, Alzheimer's disease, infertility, and numerous other conditions. In some embodiments, the desired outcome is reduction in intensity, severity, and/or frequency, and/or delay of onset of one or more of these conditions. Additionally or alternatively, Hv1 modulating agents may be used as a form of birth control by blocking sperm function.

Additionally, Hv1 modulating agents may be used to change any of the functions of Hv1 channels described in the present disclosure to achieve a preferred or therapeutic outcome. As described herein, Hv1 channels transport protons across cell membranes and are expressed in a variety of cells and tissues. Functions of Hv1 channels differ depending on the cells in which they are expressed. Uses for Hv1 modulating agents can include increasing or decreasing proton current across cell membranes and/or increasing or decreasing pH in the cytosolic, extracellular, or intraluminal space of cells.

In some embodiments, uses for Hv1 modulating agents may include effects on Hv1-related processes. For example, in some embodiments, Hv1 modulating agents may be used to increase or decrease the expression and/or function of NOX enzymes, including NOX1, NOX2, NOX3, and/or NOX4. In some embodiments, Hv1 modulating agents may be used to increase or decrease production of ROS.

In some embodiments, uses for Hv1 modulating agents may include altering biological functions in specific cells. For example, the function of Hv1 channels in white blood cells includes extrusion of protons to facilitate ROS production via NOX activity in the phagosome. This process allows white blood cells to destroy bacteria and other pathogens. In some embodiments, uses of Hv1 modulating agents may include changing these functions in white blood cells. Alternatively, Hv1 channel function in human sperm has been associated with sperm capacitation, activation and mobility to achieve fertilization. In some embodiments, Hv1 modulating agent uses may include increasing or decreasing sperm function and/or fertilization ability.

While various aspects and examples have been described, it will be apparent to those of ordinary skill in the art that many more examples and implementations are possible within the scope of this disclosure. Accordingly, the disclosure is not to be restricted except in light of the attached claims and their equivalents.

### EXAMPLES

### Example 1: Designing Hv1 Modulating Agents

The present Example describes certain Hv1 modulating agents provided herein. Certain Hv1 modulating agents provided herein comprise a polypeptide component having an amino acid sequence including element(s) found in wild-type toxin polypeptides. In some embodiments, exemplary Hv1 modulating agents comprise a polypeptide component whose amino acid sequence further comprises one or more tag elements (e.g., a detectable tag, a localizing tag, etc). In some embodiments, exemplary Hv1 modulating agents have a structure that comprises both a polypeptide component and a non-polypeptide component (e.g., a modifying component such as a lipid-containing moiety, a saccharide-containing moiety, etc). Alternatively or additionally, in some embodiments, exemplary Hv1 modulating agents are multimeric in that their structure includes multiple (e.g., 2 or more) monomer components associated with one another. In some embodiments, all monomers in a multimer are structurally identical (or substantially identical) to one another. In some embodiments, a multimer may comprise 2 or more distinct monomers. In some embodiments, two or more monomers in a multimer may be covalently associated with one another (e.g., via a linker or cross-linker).

### A. Toxin Sequences

As noted above, certain Hv1 modulating agents provided herein have an amino acid sequence including element(s) found in wild-type toxin polypeptides. Representative such agents were designed as described below.

The amino acid sequence of the Peruvian green velvet tarantula (Thrixopelma pruriens) was used as a template to identify predicted wild-type toxin sequences using the basic local alignment search tool (BLAST) in the UNIPROT™ and PFAM™ databases. A total of 110 predicted wild-type toxin sequences were identified (Table 1). All of these sequences encode a polypeptide characterized as having an inhibitor cysteine knot (ICK) structural motif. The amino acid sequences of these 110 predicted wild-type toxins were aligned around six conserved cysteine residues of the ICK motif. Three sequence elements, A, B, and C were delineated by the second and fifth of the six conserved cysteine residues in each predicted wild-type toxin sequence, resulting in 95 A, 104 B, and 106 C sequence elements (Table 3). Nucleotides were synthesized corresponding to these A, B, and C elements.

Complementary nucleotide pairs for each unique element A, B, or C, were synthesized to produce nucleotide duplexes. These nucleotide duplexes were phosphorylated using T4 Polynucleotide Kinase and annealed unidirectionally to produce polynucleotide components having an A-B-C sequence element pattern linked at cysteine residues. To achieve and monitor incorporation of the sequence elements, 104 separate reactions were performed to ligate the ABC inserts into the pAS62 phagemid vector in frame with phage particle coat protein pIII, resulting in phagemids having the ABC inserts (see, e.g., PCT/US2008/013385). Each reaction contained one B nucleotide duplex, 95 A nucleotide duplexes, and 106 C nucleotide duplexes. Ligation mixtures were transformed in SS320 electrocompetent cells (Lucigen, Middleton, WI). To verify unbiased insert utilization, 416 plaques were sequenced. The processes yielded phage particles expressing the original 110 predicted wild-type toxins and approximately 1,047,170 novel peptides. Exemplary novel peptides are listed in Table 2A and FIG. 1. Exemplary A, B and C sequence elements are listed in Table 3.

### B. Linkers and Dimerization

Certain exemplary Hv1 modulating agents were prepared by linking together two polypeptide components, each of which had an amino acid sequence comprising elements of wild-type toxin polypeptides as described above in Section A.

For example, two monomers of the agent labeled as “C6” in Table 2A were joined together via a peptide linker. Several different peptide linkers were utilized. For example, each of a rigid linker of 10 amino acid residues, a flexible linker of 10 amino acid residues, or a long flexible linker of 38 amino acid residues was used. Table 4A lists amino acid sequences of exemplary polypeptides created through such linkage.

### C. Signal Peptides

In some embodiments, one or more signal peptides can be included in exemplary Hv1 modulating agents (Table 4B).

For example, Hv1 modulating agents with an N-terminal trypsin secretory signal sequence can be prepared.

### D. Detectable Tags

In some embodiments, an Hv1 modulating agent may be modified with an epitope tag.

For example, a c-Myc epitope tag can be added near the C-terminus of a polypeptide component in an Hv1 modulating agent (Table 4C).

### E. Tethering Moieties

In some embodiments, an Hv1 modulating agent may be modified with a tethering moiety that targets the Hv1 modulating agent to a specific surface.

For example, a hydrophobic sequence may be added to the C-terminus of an Hv1 modulating agent that targets the Hv1 modulating agent for covalent tethering to glycosylphosphatidylinositol (GPI) anchors inserted in the extracellular leaflet of the plasma membrane (Table 4D).

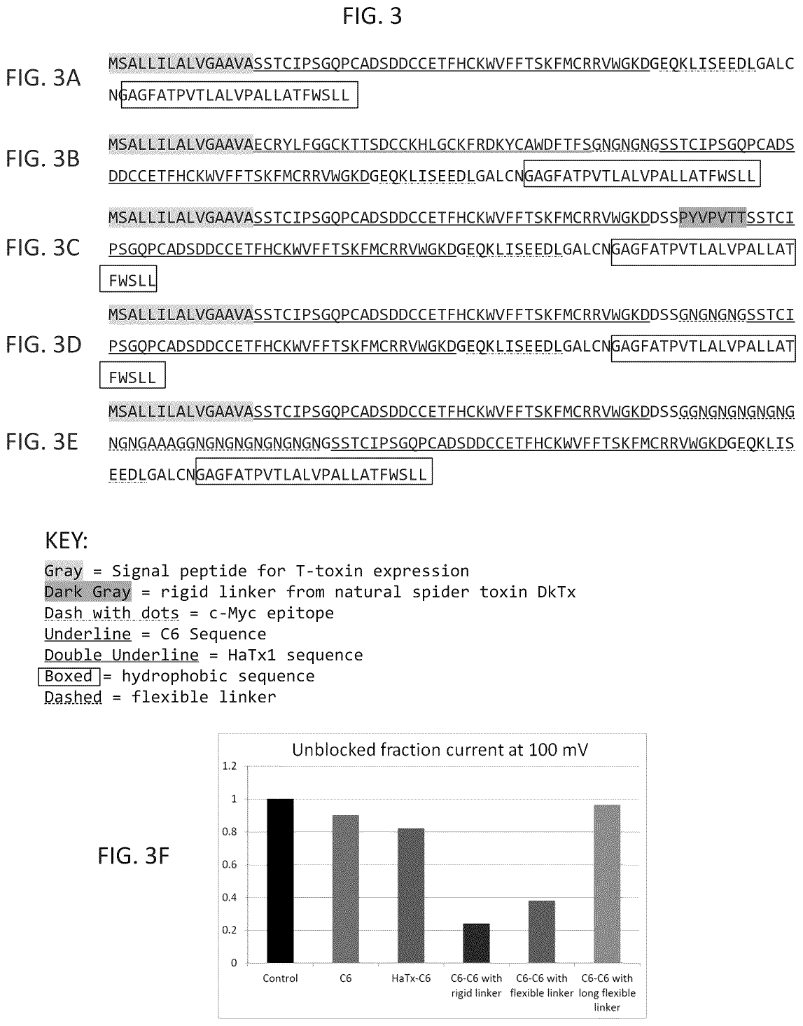

Annotated sequences of exemplary modified Hv1 modulating agents are presented in FIG. 3.

F. Generating Hv1 Modulating Agents with Linkers and Modifications

Representative Hv1 modulating agents having signal peptides, detectable tags, and tethering moieties were generated as follows. The sequence encoding mammalian Lynx1, a toxin-like nicotinic acetylcholine receptor modulator, was replaced by cDNA of the Hv1 modulating agent C6, in-frame between the secretion signal and the Lynx1 hydrophobic sequence for GPI attachment. A flexible linker containing a glycine-asparagine repeat was inserted between the C6 sequence and the hydrophobic sequence for GPI attachment, and a c-Myc epitope tag was introduced in the middle of the linker. Hv1 modulating agents having these modifications are also called “T-toxins.” Exemplary T-toxin sequences are depicted in FIGS. 3A-3E. The present disclosure appreciates that methods analogous to those described in Gui, J. et al., “A tarantula-venom peptide antagonizes the TRPA1 nociceptor ion channel by binding to the S1-54 gating domain,” Curr Biol. 24(5):473-83 (2014) can be employed to generate GPI-tethered toxins as described herein.

### Example 2: Characterization of Hv1 Modulating Agents

The present Example demonstrates a high-throughput assay for characterization of Hv1 modulating agents. Specifically, this assay characterizes whether Hv1 modulating agents are capable of binding human Hv1 (hHv1) protein.

To characterize if an Hv1 modulating agent can bind to hHv1 channels, a phage display library expressing Hv1 modulating agents was generated. Phage particles from Example 1A were used to infect Escherichia coli (E. coli) XL1-BLUE™ cells for 15 min at room temperature. The infected cells were grown overnight at 37° C. in 150 mL 2xYT in the presence of 1010/mL M13K07 helper phage, 100 μg/mL ampicillin, and 0.1 mM isopropyl β-D-1-thiogalactopyranoside (IPTG). Cultures were centrifuged and the supernatant was precipitated with PEG/NaCl solution. The phage pellet was collected by centrifugation and dissolved in TBS. Phage particle titer was determined by serial dilution in TBS and infection of E. coli XL1-Blue followed by plating on LB plates with antibiotic and determination of colony forming units (cfu).

hHv1 protein was biotinylated using sulfosuccinimidyl 2-(biotinamido)-ethyl-1,3-dithiopropionate (EZ-LINK SULFO-NHS-SS-BIOTIN™, Thermo Scientific). Biotinylation was verified by a pull-down assay using streptavidin MagneSphere beads (Promega). Biotinylated hHv1 was adsorbed to 300 μl streptavidin MagneSphere paramagnetic particles, and free streptavidin-binding sites were blocked with biotin to prevent nonspecific binding.

After manual washing of the magnetic beads, library phage particles (1011 cfu) were added in 300 μL TBSB (25 mM Tris-HCl, 140 mM NaCl, 3 mM KCl, 2 mM LPPG, 0.5% bovine serum albumin, pH 7.4) and incubated on a rocking shaker for 1 h. Poorly adherent phage particles were removed by washing 2-5 times with TBSTB (TBSB with 0.1% Tween 20). The captured phages on the magnetic beads were eluted with 100 mM DTT in 20 mM Tris, pH 8.0, for 10 min, and then used to infect E. coli XL1-Blue cells (Stratagene) for phage amplification. The phage particles captured in the first round were cycled through an additional five rounds of binding and selection using an automated magnetic bead manipulator (KINGFISHER™, Thermo Scientific). Phage particles were quantified by titering before and after selective library sorting and genotyped by DNA sequencing after six rounds of panning. Phage enrichment was observed with immobilized hHv1 as the target compared with the control target streptavidin. Exemplary Hv1 modulating agents enriched by this method are listed in Table 2A. In some instances, repeats of Hv1 modulating agent sequences after several rounds of panning can be observed, as demonstrated by agents labeled as A6 and G2, C2 and F2, C6 and D5, and D6 and E2. Without wishing to be bound by any particular theory, any repeat may be considered significant (since the library had more than 1 million peptides initially) and may demonstrate selection and functional convergence.

### Example 3: Characterization of Hv1 Modulating Agents by T-Toxin Assay

The present example demonstrates characterization of the effects of Hv1 modulating agents on hHv1 using T-toxins. Specifically, the present example demonstrates inhibition of hHv1 function as measured by tail current from Xenopus laevis oocytes expressing only hHv1 or both hHv1 and Hv1 modulating agents tethered to the oocyte plasma membrane.

T-toxin cDNAs were cloned into the pCS2+ plasmid vector for in vitro transcription of T-toxin cRNA. Capped cRNAs were prepared by restriction enzyme linearization, followed by in vitro transcription reaction with SP6 (for T-toxins) and T7 (for hHv1) RNA polymerase (mMessenger mMachine kit, Ambion). Concentrations of cRNAs were measured using NanoDrop 2000 (Thermo Scientific).

cRNAs for T-toxins and hHv1 were mixed at 1:1 ratio (w/w) and injected into the Xenopus laevis oocytes. Currents were measured by Two Electrode Voltage Clamp (TEVC). Recording solution was (in mM): 90 NaCl, 1 MgCl₂, 2 CaCl₂), 120 HEPES, pH 7.3. Recordings were performed with constant gravity flow of solution at 2 ml/min yielding chamber exchange in ˜5 s. Currents were recorded 2-3 days after cRNA injection using an oocyte clamp amplifier OC-725C (Warner Instruments, Hamden, CT), and electrodes filled with 3 μM KCl with resistance of 0.3-1 MΩ. Data were filtered at 1 kHz and digitized at 20 kHz using pClamp software and assessed with Clampfit v9.0 and Origin 6.0. Inhibition was studied by comparing tail current from oocytes expressing only hHv1 and those with both hHv1 and T-toxins. Inhibition was calculated as unblocked fractional current (FIG. 3F).

### Example 4: Characterization of Hv1 Modulating Agents with a Transmembrane Link

The present example demonstrates characterization of Hv1 modulating agent effects on hHv1 using Hv1 modulating agents expressed from cells via a transmembrane link.

The Hv1 modulating agent C6 was tethered to cell surfaces using a transmembrane domain from the PDGF receptor, which links an internal mVenus fluorescent protein to an external C6 (FIG. 7). Unlike the tether in oocytes which attaches C6 to the outside of the cell (e.g. FIG. 3), this is a transmembrane link. To transiently express hHv1, 1 g of hHv1 tagged with teal fluorescence protein (hHv1-TFP) was transfected into HEK-293T cells using Lipofectamine 2000. Changes in the single exponential fit of fluorescence decay were indicative of FRET (FIG. 8). Changes in current density and shifts in the g_{H}-V were indicative of blocking of hHv1.

### Example 5: Hv1 Modulating Agents Activate or Inhibit Hv1 Proton Current

The present Example documents assessing activity of the voltage-gated proton channel Hv1 in response to Hv1 modulating agents. Activity of Hv1 was assessed by changes in proton currents.

To transiently express hHv1, 1 g of hHv1 or hHv1 tagged with teal fluorescence protein (hHv1-TFP) was transfected into HEK-293T cells using Lipofectamine 2000. In some instances, green fluorescent protein was used as a transfection marker. Transfected cells were plated onto glass coverslips. Green or teal cells were selected for whole-cell patch clamp after 24 hours.

Patch clamp recordings were performed with an external solution of 100 mM HEPES, pH 7.5, 70 mM NaCl, and 10 mM glucose. The pipette solution was 100 mM Bis Tris buffer, pH 6.5, 70 mM NaCl and 10 mM glucose. Hv1 modulating agent was applied to these cells while pulsing to 40 mV every 10 seconds and proton currents were measured using whole-cell patch clamp electrophysiology.

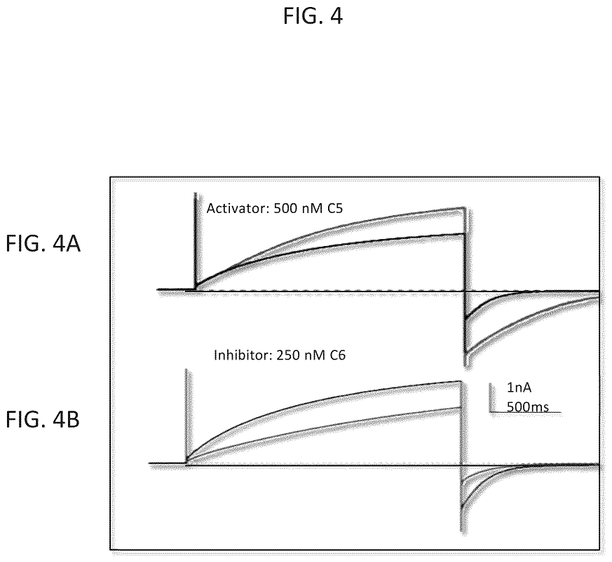

FIG. 4 shows activation or inhibition of Hv1 proton currents by Hv1 modulating agent as compared to current without addition of modulating agent. Cells were incubated with either 500 nM C5 (FIG. 4A) or 250 nM C6 (FIG. 4B). Activation of Hv1 channels by C5 increased proton current and slowed channel closing. Block of Hv1 channels by C6 decreased proton currents.

### Example 6: Hv1 Modulating Agent Inhibits Effects of Progesterone on Sperm Capacitation

The present example demonstrates effectiveness of certain Hv1 modulating agents in suppressing maturation of the sperm response to progesterone. This example shows that activity of Hv1 channels during capacitation is necessary for calcium rise and acrosomal reaction stimulation by physiological inducers required for fertilization.

To test effects of Hv1 modulating agents on sperm capacitation in a blind study, human sperm was exposed to capacitating conditions in the presence or absence of Hv1 modulating agent C6 (Tx C) or a control peptide (Tx A) having the amino acid sequence GVEINVKCSGSPQCLKPCKDAGMDFGDCMNDKCHCTPK (SEQ ID NO: 810) (a mutant scorpion venom peptide; Takacs, Z., et al., “A designer ligand specific for Kv1.3 channels from a scorpion neurotoxin-based library,” Proc Natl Acad. Sci. USA. 106(52): 22211-22216 (2009)). Sperm were incubated for 1 hour with the Tx C or Tx A (20 M) in a medium that does not promote capacitation. Cells were transferred to a capacitating medium with Tx C or Tx A. After 4 hours of incubation, parameters related to capacitation were analyzed (FIG. 5). Known protocols for such analyses are described in Pocognoni, C. A., et al., “Perfringolysin O as a useful tool to study human sperm physiology,” Fertility and Sterility, 99(1): p. 99-106.e2 (2013).

C6 did not affect the vitality (FIG. 5A), the protein tyrosine phosphorylation (FIG. 5F and FIG. 5G), or the cholesterol content of the membranes (FIG. 5H). C6 did not significantly alter the mobility of sperm (FIGS. 5B-5E). C6 did affect the response of sperm to progesterone. The increase of cytosolic calcium triggered by progesterone is diminished in the presence of the peptide modulator and the acrosome reaction induced by the hormone is inhibited (FIGS. 5I-5K). Moreover, when C6 was added after capacitation, both intracellular calcium and acrosomal reaction, triggered by progesterone, did not show any changes.

### Example 7: Hv1 Modulating Agents Inhibit Production of Reactive Oxygen Species in Human Blood Cells

The present example demonstrates effectiveness of certain Hv1 modulating agents in blocking ROS production by human blood cells.

Whole blood was purchased in 10 mL tubes from Innovative Research, Inc, in accordance with FDA guidelines. Blood was used within 24-48 hours after being drawn. Upon arrival, blood cells were counted using a hemocytometer and diluted in Tyrode's solution to approximately 5×10⁶ cells/mL. Twenty L of the dilute blood cells were added to each well of a 96-well plate with a total volume of 100 μL in Tyrode's solution. Reactive oxygen species (ROS) were detected by fluorescence readout using 100 μM Amplex Red with 2 units/mL horseradish peroxidase added to each well.

Blood was incubated with the following treatment conditions for 1 hour at 37 C: control; 100 μM zinc; 100 μM-5 μM C6; and 10 μM MOKA toxin. Each treatment condition was added to wells in 5 repeats. After the incubation, 200 nM phorbol myristate acetate (PMA) was added to all wells except the control. PMA was used to stimulate ROS production. Fluorescence measurements were taken immediately after using excitation at 530 nM and emission at 590 nM. Measurements were repeated every 15 to 30 min for the next 2 hours. Relative fluorescence intensity was plotted versus time and used to calculate inhibition. FIG. 6 demonstrates that C6 blocks production of ROS in human blood cells in a dose-dependent manner. The known inhibitor of Hv1, zinc, blocks ROS production to background (control) levels. Two toxins that block potassium channels with nM affinity (Moka and KTX) had no effect.

### Example 8: The Hv1 Modulating Agent C6 Targets an S3-S4 External Loop Region of hHv1

The present example demonstrates identification of regions in hHv1 that can bind and/or respond to modulation by the Hv1 modulating agent C6.

The Hv1 modulating agent C6 did not inhibit proton current of Ciona intestinalis (C. intestinalis) Hv1 channels (CiHv1). Chimeric forms of ciHv1 were generated in which amino acids from human Hv1 (hHv1) corresponding to the S3-S4 external loop replaced the same region of CiHv1 (hS3-S4-ciHv1). The resulting hS3-S4-ciHv1 chimera comprised hHv1 amino acids 1183 to L204: ILDIVLLFQEHQFEALGLLILL (SEQ ID NO: 111) and maintained characteristics of Hv1 currents. C6 blocked current for hS3-S4-ciHv1 (FIGS. 9A and 9B). A different chimeric form of ciHv1 (shortC), comprised hHv1 amino acids 1183 to F195: ILDIVLLFQEHQF (SEQ ID NO: 112). The shortC chimera had very low currents (approximately 100 pA), and was responsive to C6 inhibition (FIG. 9A).

Twelve residues in the S3-S4 external loop region of hHv1 (F190 to L201) were individually mutated to Cysteine. Current with 1 μM C6 normalized to current without toxin (Itox/Ictr) was measured (FIG. 9C). Mutating hHv1 E192C increased normalized current with C6 compared to WT hHv1. Mutating hHv1 G199C or G199 μL increased inhibitory effects of C6 compared to WT hHv1.

Equivalents: Those skilled in the art will recognize, or be able to ascertain using no more than routine experimentation, many equivalents to the specific embodiments of the invention described herein. The scope of the present invention is not intended to be limited to the above Description, but rather is as set forth in the following claims.

1-80. (canceled)

81: A method comprising the step of administering to a subject in need thereof suffering from or susceptible to an HvI associated disease or condition a pharmaceutical composition comprising a voltage-gated proton channel (Hv1) inhibitor comprising:
(a) at least two polypeptide components, wherein each component has an amino acid sequence comprising: (SEQ ID NO:129); or
(b) a polypeptide having amino acid sequence of SEQ ID NO:128; or
(c) a polypeptide having amino acid sequence of SEQ ID NO:129,
wherein administering the pharmaceutical composition to the subject treats the HvI associated disease or condition or reduces the risk of developing an HvI associated disease or condition.

82: The method of claim 81, wherein the HvI associated disease or condition is selected from the group consisting of: inflammation, allergies, autoimmunity, cancer, asthma, brain damage from ischemic stroke, Alzheimer's disease and infertility.

83: The method of claim 82, wherein the cancer is a leukemia or a solid tumor.

84: The method of claim 83, wherein the solid tumor is breast cancer.

85-91. (canceled)

92: The method of claim 81, wherein at least one of the at least two polypeptide components comprise one or more modifications selected from the group consisting of acetylation, amidation, glycosylation, lipidation, methylation, pegylation, phosphorylation, and combinations thereof.

93: The method of claim 81, wherein the polypeptide components are linked together via a linker.

94: The method of claim 93, wherein the polypeptide components are linked together via a peptide linker.

95: The method of claim 93, wherein the linker comprises a rigid or flexible linker.

96: The method of claim 81, wherein the Hv1 inhibitor is fused to an antibody sequence or fragment thereof.

97: The method of claim 81, wherein the pharmaceutical composition further comprises a pharmaceutically acceptable carrier or excipient.

98: The method of claim 81, wherein the polypeptide components are covalently linked together.

99: The method of claim 81, wherein the Hv1 associated disease or condition is inflammation.

100: The method of claim 81, wherein the Hv1 associated disease or condition is an allergy.

101: The method of claim 81, wherein the HvI associated disease or condition is disease or condition having an autoimmunity component.

102: The method of claim 81, wherein the HvI associated disease or condition is cancer.

103: The method of claim 81, wherein the HvI associated disease or condition is asthma.

104: The method of claim 81, wherein the HvI associated disease or condition is brain damage from ischemic stroke.

105: The method of claim 81, wherein the HvI associated disease or condition is Alzheimer's disease.

106: The method of claim 81, wherein the HvI associated disease or condition is infertility.
